(19) United States
(12) Patent Application Publication
(10) Pub. No.: US 2024/0325456 A1
(43) Pub. Date: Oct. 3, 2024

(54) MULTIPOTENT LUNG PROGENITOR CELLS FOR LUNG REGENERATION

(71) Applicants: Yeda Research and Development Co. Ltd., Rehovot (IL); Board of Regents, The University of Texas System, Austin, TX (US)

(72) Inventors: Yair REISNER, Houston, TX (US); Chava ROSEN, Rehovot (IL); Elias SHEZEN, Rehovot (IL); Ran ORGAD, Rehovot (IL); Irit MILMAN KRENTSIS, Rehovot (IL); Xiaohua SU, Houston, TX (US); Raj Kumar YADAV, Houston, TX (US); Yangxi ZHENG, Houston, TX (US)

(73) Assignees: Yeda Research and Development Co. Ltd., Rehovot (IL); Board of Regents, The University of Texas System, Austin, TX (US)

(21) Appl. No.: 18/736,635

(22) Filed: Jun. 7, 2024

Related U.S. Application Data
(63) Continuation of application No. PCT/IL2022/051302, filed on Dec. 8, 2022.
(60) Provisional application No. 63/287,147, filed on Dec. 8, 2021.

(51) Int. Cl. A61K 35/42; C12N 5/071

(52) U.S. Cl. CPC A61K 35/42; C12N 5/0688; C12N 2501/11; C12N 2501/165; C12N 2501/727; C12N 2509/00

(57) ABSTRACT

Multipotent lung progenitor cells for lung regeneration are provided. Accordingly, there are provided methods of expanding in culture an isolated population of pulmonary cells, methods of qualifying suitability of an isolated population of pulmonary cells for administration to a subject in need thereof, and methods of generating an isolated population of pulmonary cells comprising selecting a cell population being double positive for expression of epithelial and endothelial cell markers. Also provided are isolated populations of pulmonary cells, pharmaceutical compositions comprising same and uses of same.

### RELATED APPLICATIONS

This application is a Continuation of PCT Patent Application No. PCT/IL2022/051302 having International filing date of Dec. 8, 2022, which claims the benefit of priority under 35 USC § 119 (e) of U.S. Provisional Patent Application No. 63/287,147 filed on Dec. 8, 2021. The contents of the above applications are all incorporated by reference as if fully set forth herein in their entirety.

### FIELD AND BACKGROUND OF THE INVENTION

The present invention, in some embodiments thereof, relates to pulmonary progenitor cells and, more particularly, but not exclusively, to methods of generating same and use of same in therapeutic applications.

End stage respiratory diseases are among the leading causes of death worldwide, with more than 5.5 million deaths annually (World Health Organization data for 2020). Today, the only definitive treatment for these conditions is by replacement of the damaged organ with a lung transplant. Due to a shortage of suitable organs, many patients die on the transplant waiting list, and therefore lung diseases are prime candidates for stem cell therapy.

Various cell populations have been shown to exhibit regenerative potential, including BM-derived cells (1), lung-derived p63+ cells (2), LNEP (lineage negative epithelial progenitors) (3), and mouse and human sox9+ cells (4), (5). Recently, fetal lung progenitors were suggested as an attractive source for transplantation in mice, provided that the lung stem cell niche in the recipient is vacated of endogenous lung progenitors by adequate conditioning. Thus, in a procedure akin to bone marrow transplantation (BMT), a single cell suspension of mouse or human fetal lung cells harvested at the canalicular phase of gestation (20-22 weeks in human, and E15-E16 for mouse) and infused I.V. following conditioning of recipient mice with naphthalene and 6Gy TBI, led to marked lung chimerism within alveolar and bronchiolar lineages. This chimerism was associated with significantly improved lung function (6). More recently, marked lung chimerism was extended to transplantation of a single cell suspension of adult mouse lung donors (7) (8), requiring about three fold higher cell doses to attain a similar level of chimerism of that found following fetal cell transplantation (6).

Additional background art includes:
PCT publication no. WO/2013/084190 discloses a pharmaceutical composition comprising as an active ingredient an isolated population of cell suspension from a mammalian fetal pulmonary tissue, the fetal pulmonary tissue is at a developmental stage corresponding to that of a human pulmonary organ/tissue at a gestational stage selected from a range of about 20 to about 22 weeks of gestation.
PCT publication no. WO/2016/203477 discloses a method of conditioning a subject in need of transplantation of progenitor cells in suspension of a tissue of interest.
PCT publication no. WO/2017/203520 discloses a method of treating a pulmonary disorder or injury comprising administering to the subject non-syngeneic pulmonary tissue cells in suspension comprising an effective amount of hematopoietic precursor cells (HPCs) or supplemented with HPCs, wherein the effective amount is a sufficient amount to achieve tolerance to the pulmonary tissue cells in the absence of chronic immunosuppressive regimen.

### SUMMARY OF THE INVENTION

According to an aspect of some embodiments of the present invention there is provided a method of expanding in culture an isolated population of pulmonary cells, the method comprising:
(a) dissociating a pulmonary tissue so as to obtain a population of isolated pulmonary cells; and
(b) expanding the population of isolated pulmonary cells in a medium comprising a factor that promotes expansion of endothelial cells, a factor that promotes expansion of epithelial cells, and a factor that prevents differentiation, so as to expand a cell population being double positive for expression of epithelial and endothelial cell markers,
thereby expanding in culture the isolated population of pulmonary cells.

According to an aspect of some embodiments of the present invention there is provided a method of qualifying suitability of an isolated population of pulmonary cells for administration to a subject in need thereof, the method comprising:
(a) dissociating a pulmonary tissue so as to obtain a population of isolated pulmonary cells;
(b) expanding the population of isolated pulmonary cells in a culture; and
(c) determining expression of epithelial and endothelial cell markers on the population of isolated pulmonary cells during and/or following the culture,
wherein expansion above a predetermined threshold of a cell population being double positive for expression of the epithelial and endothelial cell markers indicates the population of isolated pulmonary cells is suitable for administration to the subject; and
wherein no expansion or expansion below the predetermined threshold of the cell population being double positive for expression of the epithelial and endothelial cell markers indicates the population of isolated pulmonary cells is not suitable for administration to the subject,
thereby qualifying suitability of the isolated population of pulmonary cells for administration to the subject.

According to an aspect of some embodiments of the present invention there is provided a method of generating an isolated population of pulmonary cells, the method comprising: (a) dissociating a pulmonary tissue so as to obtain a population of isolated pulmonary cells; and (b) contacting the population of isolated pulmonary cells with at least one agent capable of binding an epithelial cell marker and an endothelial cell marker, so as to select a cell population being double positive for expression of epithelial and endothelial cell markers, thereby generating the isolated population of pulmonary cells.

According to some embodiments of the invention, the method further comprising expanding the pulmonary cells in a culture following step (b).

According to some embodiments of the invention, the culture medium comprises a factor that promotes expansion of endothelial cells, a factor that promotes expansion of epithelial cells, and a factor that prevents differentiation. According to some embodiments of the invention, the method further comprising determining expression of the epithelial and endothelial cell markers on the pulmonary cells during and/or following the culture.

According to some embodiments of the invention, wherein expansion above a predetermined threshold of the cell population being double positive for expression of the epithelial and endothelial cell markers indicates the population of isolated pulmonary cells is suitable for administration to a subject in need thereof; and wherein no expansion or expansion below the predetermined threshold of the cell population being double positive for expression of the epithelial and endothelial cell markers indicates the population of isolated pulmonary cells is not suitable for administration to the subject.

According to some embodiments of the invention, the factor that promotes expansion of endothelial cells is selected from the group consisting of vascular endothelial growth factor (VEGF), FGF, FGF2, IL-8 and BMP4.

According to some embodiments of the invention, the factor that promotes expansion of endothelial cells comprises vascular endothelial growth factor (VEGF).

According to some embodiments of the invention, the factor that promotes expansion of epithelial cells is selected from the group consisting of epidermal growth factor (EGF), Noggin and

R-Spondin.

According to some embodiments of the invention, the factor that promotes expansion of epithelial cells comprises epidermal growth factor (EGF).

According to some embodiments of the invention, the factor that prevents differentiation is selected from the group consisting of a ROCK inhibitor, a GSK3b inhibitor and an ALK5 inhibitor.

According to some embodiments of the invention, the factor that prevents differentiation comprises a ROCK inhibitor.

According to an aspect of some embodiments of the present invention there is provided an isolated population of pulmonary cells comprising at least 40% CD326+CD31+ cells.

According to an aspect of some embodiments of the present invention there is provided an isolated population of pulmonary cells obtained according to the method of some embodiments of the invention.

According to an aspect of some embodiments of the present invention there is provided a pharmaceutical composition comprising as an active ingredient the isolated population of pulmonary cells of some embodiments of the invention and a pharmaceutical acceptable carrier.

According to an aspect of some embodiments of the present invention there is provided a method of regenerating an epithelial and/or endothelial pulmonary tissue in a subject in need thereof, the method comprising administering to the subject a therapeutically effective amount of the isolated population of pulmonary cells of some embodiments of the invention, thereby regenerating the epithelial and/or endothelial pulmonary tissue.

According to an aspect of some embodiments of the present invention there is provided a method of treating a pulmonary disorder or injury in a subject in need thereof, the method comprising administering to the subject a therapeutically effective amount of the isolated population of pulmonary cells of some embodiments of the invention, thereby treating the pulmonary disorder or injury.

According to an aspect of some embodiments of the present invention there is provided a therapeutically effective amount of the isolated population of pulmonary cells of some embodiments of the invention for use in treating a pulmonary disorder or injury in a subject in need thereof.

According to an aspect of some embodiments of the present invention there is provided a kit for isolation of pulmonary cells characterized as being double positive for expression of epithelial and endothelial cell markers, the kit comprising: (I) at least one agent capable of binding: (i) CD31 or CD144; and (ii) CD326, CD324, CD24, Aquaporin 5 (AQP-5), Podoplanin (PDPN), or Advanced Glycosylation End-Product Specific Receptor (RAGE); and (II) instructions for use.

According to an aspect of some embodiments of the present invention there is provided a cell bank comprising: (i) a plurality of isolated populations of pulmonary cells in suspension, the pulmonary cells being characterized as double positive for the expression of epithelial and endothelial cell markers, and wherein the plurality of the isolated populations of the pulmonary cells have been HLA typed to form an allogeneic cell bank, each individually disposed within separate containers; and (ii) a catalogue which comprises information about the HLA typed cells of the plurality of the isolated populations of the pulmonary cells.

According to some embodiments of the invention, the epithelial cell marker comprises CD326, CD324, CD24, Aquaporin 5 (AQP-5), Podoplanin (PDPN), or Advanced Glycosylation End-Product Specific Receptor (RAGE).

According to some embodiments of the invention, the endothelial cell marker comprises CD31 or CD144 (VE-cadherin).

According to some embodiments of the invention, the cell population being double positive for expression of epithelial and endothelial cell markers comprises a CD326⁺CD31⁺ signature.

According to some embodiments of the invention, the cell population being double positive for expression of epithelial and endothelial cell markers comprises a CD324⁺CD31⁺ signature.

According to some embodiments of the invention, the cell population being double positive for expression of epithelial and endothelial cell markers comprises a CD326⁺CD144⁺ signature.

According to some embodiments of the invention, the cell population being double positive for expression of epithelial and endothelial cell markers comprises a CD324⁺CD144⁺ signature.

According to some embodiments of the invention, the method further comprises depleting CD45 expressing cells.

According to some embodiments of the invention, the depleting CD45 expressing cells is affected by contacting the population of isolated pulmonary cells with an agent capable of binding CD45, so as to select a cell population being negative for expression of CD45.

According to some embodiments of the invention, the method further comprises depleting T cells.

According to some embodiments of the invention, the method further comprises expanding the pulmonary cells in a culture following step (b).

According to some embodiments of the invention, the at least one agent capable of binding is an antibody.

According to some embodiments of the invention, the antibody is a monospecific antibody.

According to some embodiments of the invention, the antibody is a bispecific antibody.

According to some embodiments of the invention, the dissociating is by enzymatic digestion.

According to some embodiments of the invention, the method is affected ex vivo.

According to some embodiments of the invention, the pulmonary tissue is a fetal pulmonary tissue.

According to some embodiments of the invention, the pulmonary tissue is an adult pulmonary tissue.

According to some embodiments of the invention, the pulmonary tissue is a human pulmonary tissue.

According to some embodiments of the invention, the pulmonary tissue is from a cadaver donor.

According to some embodiments of the invention, the pulmonary tissue is from a living donor.

According to some embodiments of the invention, the pulmonary cells are capable of regenerating an epithelial pulmonary tissue.

According to some embodiments of the invention, the pulmonary cells are capable of regenerating an endothelial pulmonary tissue.

According to some embodiments of the invention, the cells are in suspension.

According to some embodiments of the invention, the cells are embedded or attached to a scaffold.

According to some embodiments of the invention, the pharmaceutical composition further comprises as an active ingredient hematopoietic precursor cells (HPCs).

According to some embodiments of the invention, the HPCs comprise T cell depleted immature hematopoietic cells.

According to some embodiments of the invention, the method further comprises administering to the subject an agent capable of inducing damage to a pulmonary tissue prior to the administering, wherein the damage results in proliferation of resident stem cells in the pulmonary tissue.

According to some embodiments of the invention, the method further comprises conditioning the subject under sublethal, lethal or supralethal conditioning protocol prior to the administering.

According to some embodiments of the invention, the method further comprises administering to the subject an effective amount of hematopoietic precursor cells (HPCs).

According to some embodiments of the invention, the method further comprises treating the subject with an immunosuppressive agent following the administering.

According to some embodiments of the invention, the isolated population of pulmonary cells for use further comprises the use of an agent capable of inducing damage to a pulmonary tissue, wherein the damage results in proliferation of resident stem cells in the pulmonary tissue.

According to some embodiments of the invention, the isolated population of pulmonary cells for use further comprises a sublethal, lethal or supralethal conditioning protocol.

According to some embodiments of the invention, the isolated population of pulmonary cells for use further comprises the use of an effective amount of hematopoietic precursor cells (HPCs).

According to some embodiments of the invention, the isolated population of pulmonary cells for use further comprises the use of an immunosuppressive agent.

According to some embodiments of the invention, the agent capable of inducing damage to the pulmonary tissue is selected from the group consisting of a chemotherapeutic agent, an immunosuppressive agent, an amiodarone, a beta blockers, an ACE inhibitor, a nitrofurantoin, a procainamide, a quinidine, a tocainide, and a minoxidil.

According to some embodiments of the invention, the agent capable of inducing damage to the pulmonary tissue comprises naphthalene.

According to some embodiments of the invention, the conditioning protocol comprises reduced intensity conditioning (RIC).

According to some embodiments of the invention, the conditioning protocol comprises at least one of total body irradiation (TBI), partial body irradiation, a chemotherapeutic agent and/or an antibody immunotherapy.

According to some embodiments of the invention, the antibody immunotherapy comprises T cell debulking.

According to some embodiments of the invention, the antibody immunotherapy comprises anti-thymocyte globulin (ATG) antibody, alemtuzumab, muromonab-CD3, or a combination thereof.

According to some embodiments of the invention, the TBI comprises a single or fractionated irradiation dose within the range of 1-10 Gy.

According to some embodiments of the invention, the HPCs comprise pulmonary tissue-derived CD34+ cells.

According to some embodiments of the invention, the HPCs comprise bone marrow or mobilized peripheral blood CD34+ cells.

According to some embodiments of the invention, the HPCs comprise T cell depleted immature hematopoietic cells.

According to some embodiments of the invention, the isolated population of pulmonary cells and the HPCs are obtained from the same donor.

According to some embodiments of the invention, the isolated population of pulmonary cells and the hematopoietic precursor cells (HPCs) are in separate formulations.

According to some embodiments of the invention, the isolated population of pulmonary cells and the HPCs are in the same formulation.

According to some embodiments of the invention, the isolated population of pulmonary cells and/or the HPCs are formulated for an intravenous or an intratracheal route of administration.

According to some embodiments of the invention, the immunosuppressive agent comprises cyclophosphamide, busulfan, fludarabin, tacrolimus, cyclosporine, mycophenolate mofetil, azathioprine, everolimus, sirolimus, glucocorticoids, or combinations thereof.

According to some embodiments of the invention, the subject is a human subject.

According to some embodiments of the invention, the isolated population of pulmonary cells is non-syngeneic with the subject.

According to some embodiments of the invention, the pulmonary disorder or injury comprises chronic inflammation of the lungs.

According to some embodiments of the invention, the pulmonary disorder or injury is selected from the group consisting of cystic fibrosis, emphysema, asbestosis, chronic obstructive pulmonary disease (COPD), pulmonary fibrosis, idiopatic pulmonary fibrosis, pulmonary hypertension, lung cancer, sarcoidosis, acute lung injury (adult respiratory distress syndrome), respiratory distress syndrome of prematurity, chronic lung disease of prematurity (bronchopulmonarydysplasia), surfactant protein B deficiency, congenital diaphragmatic hernia, pulmonary alveolar proteinosis, pulmonary hypoplasia and asthma.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F demonstrate multi-lineage engraftment of mTom donor-derived cells in the recipient lung evaluated by sc-RNA seq. FIG. 1A shows the design of cell sorting of host and donor lung cells from chimeric lungs. Three chimeric lungs were first verified to exhibit significant chimerism by fluorescent microscopy, pooled, enzymatically dissociated and FACS separated after gating on CD45−, single, live cells into donor and recipient compartments based on Td-Tomato expression. FIG. 1B shows FACS analysis of chimeric lung, transplanted with Td-Tomato cells and control non-transplanted lung. FIGS. 1C-D show transcriptome analysis of FACS sorted donor (FIG. 1C) and recipient (FIG. 1D)-derived compartments at 6 months posttransplant. Monocle 3 UMAPs and proportional plots of the clusters demonstrate analysis of n=6081 donor and n=3210 recipient cells, identifying major epithelial and endothelial clusters within donor derived compartment comparable with the clusters defined in the recipient derived compartment. FIG. 1E-F show heat maps identifying the functionally distinct gCap and aCap endothelial cells in donor (FIG. 1E) and recipient (FIG. 1F) compartments, respectively.

FIGS. 2A-E demonstrate transplantation of fetal lung cells from Confetti donors. FIG. 2A shows the experimental scheme using E16 fetal lung cells after induction of CRE recombination by Tamoxifen for implantation into immune deficient RAG recipients preconditioned with naphthalene and 6Gy TBI. FIG. 2B shows FACS analysis of E16 R26R-Confetti fetal lung, demonstrating the percentage of Cre recombined cells, n=5 embryos. FIG. 2C is a two photon microscopy image of E16 fetal lung tissue, illustrating single monochromatic cells in R26R-Confetti mouse after TMX induction and prior to transplantation (from n=3 embryos). FIG. 2D shows monochromatic patches at 6 weeks after transplantation of E16 fetal lung cells from R26R-Confetti donors into RAG2−/− recipient mouse. Whole mount of the chimeric lung was analyzed by fluorescent microscopy under low magnification (scale bar=200 μm). FIG. 2E shows typical analysis of the same chimeric lung by two photon microscopy showing monochromatic patches expressing different fluorescence tags (Scale bar=50 μm). The results are representative of 3 independent experiments, n=3 mice in each experiment.

FIGS. 3A-G demonstrate lung chimerism analysis at 8 weeks after transplantation of adult R26R-Confetti lung cells. FIG. 3A is a schematic presentation of the experimental procedure. FIG. 3B, left image, is a two photon microscopy image of the adult lung prior to transplantation revealing single cells expressing one of the four tag colors after Cre recombination; n=3. FIG. 3B, right image, is a confocal image of adult R26R-Confetti lung slice demonstrating bronchial and alveolar parts of the lung tissue, and diffuse and random localization of the fluorescent cells within the donor lung. The results are representative of n=6 R26R-Confetti mice from 2 independent experiments. FIG. 3C-D show FACS analysis of adult R26R-Confetti lung, after administration of two doses of TMX, demonstrating percentages of Cre recombined cells, bearing different fluorescent tags, n=5. In FIGS. 3E-F transparent chimeric lung by a clearing procedure was rendered and analyzed with light sheet microscope. A total of n=2 chimeric lungs were evaluated by LSM. Scale bar=60 μm in FIG. 3E and 40 μm in FIG. 3F. FIG. 3G shows typical monochromatic patches, each exhibiting distinct color in whole mount chimeric lung. In total n=4 chimeric lungs were evaluated by two-photon microscopy. Scale bar=50 μm.

FIGS. 4A-H demonstrate donor derived lung patches after transplantation of different lung cell sub-populations. FIG. 4A shows the gating strategy for FACS sorting of CD45-lung cells into four subpopulations including CD326+CD31−, CD326+CD31+, CD326−CD31+ and CD326−CD31− cells. FIG. 4B shows visualization of double positive CD45−CD326+CD31+ lung cells by Imagestream analysis. FIG. 4C is a schematic representation of the transplantation experiments. FIG. 4D shows the percentage of each sorted subpopulation out of the CD45-non-hematopoietic lung cell population in 16 experiments. FIG. 4E shows the percentage of chimeric mice exhibiting donor-derived patches out of the total number of transplanted mice (n=16 experiments). FIG. 4F shows donor-derived lung patches 6 weeks after transplantation of 0.3-0.5×10⁶ sorted double positive CD326+CD31+ cells, or single positive CD31+ endothelial cells from nTnG donors (red) mixed with 0.5×10⁶ unsorted cells from GFP+ donors (green). The whole mount confocal images shown for each group are representative of n=16 experiments, with at least n=10 mice in each group; scale bar=500 μm. Out of the total transplanted mice (n=98), 70% were transplanted with sorted td-tomato+ unsorted GFP cells, and 30% were transplanted only with the FACS td-tomato sorted cells. FIG. 4G-H show representative images of whole mount lungs of mice transplanted with 0.5×10⁶ nTnG CD326+CD31+ or CD31+ cells, in the absence of GFP+co-transplanted unsorted lung cells. Donor-derived red patches express nuclear td-tomato. Images under low (scale bar=500 μm) and high magnification (scale bar=100 μm) are representative of n=5 mice in each group.

FIGS. 5A-K demonstrate different cellular composition of donor derived patches after transplantation of sorted CD326-CD31+ versus CD326+CD31+ lung cell subpopulations. FIG. 5A-D show staining of typical lung patches derived from sorted nTnG CD326-CD31+ lung cells (red). FIGS. 5A-B shows staining for endothelial markers ERG (nuclear; green); scale bar=100 μm (FIG. 5A) and 10 μm (FIG. 5B). FIG. 5C shows staining for endothelial nuclear marker SOX17 (cyan) within donor-derived nTnG positive patch after transplantation of CD326-CD31+ cells. Markers stained are indicated on the images; scale bar=10 μm. FIG. 5D shows staining for cell surface endothelial marker CD31 and epithelial marker HOPX in donor-derived patches formed after transplantation of CD326-CD31+ cells. On the left-double staining for CD31 and SOX17; on the right-Triple staining for nTnG+ (red), CD31 (blue) and HOPX (green). Donor-derived endothelial CD31+ cells reside in close proximity to recipient HOPX+ AT1 cells (indicated with arrow heads), while donor derived AT1 cells cannot be detected; scale bar=10 μm. FIGS. 5E-F show staining of typical lung patches derived from sorted nTnG CD326+CD31+ lung progenitor cells, demonstrating donor-derived epithelial and endothelial cells. FIG. 5E, left panel: double staining for nTnG (red) and SOX17 (cyan); middle panel: single staining for SOX17; right panel: Triple staining for nTnG (red), SOX17 (cyan) and HOPX (green), showing donor-derived AT1 epithelial cells (arrowheads) and endothelial cells (arrows); scale bar=15 μm. FIG. 5F shows high magnification of typical staining for HOPX demonstrating donor (red, indicated with arrow) and host (indicated with arrow head) AT1 cells. FIG. 5G shows high magnification of triple staining of CD326+CD31+-derived patch, demonstrating the presence of donor-derived (red) AT1 and endothelial CD31+ cells, in close proximity. Arrow indicates donor-derived AT1 cells, and arrowhead indicates the host-derived AT1 cells. Scale bar=10 μm in FIGS. 5F and 5G. Images are representative of n=3 mice. FIG. 5H shows staining of a typical lung patch derived from sorted nTnG CD326+CD31+ lung cells demonstrating donor-derived epithelial AT1 (AQP-5+, purple) and endothelial (SOX17+, green) cells. Left panel: double staining nTnG (red) and SOX17 (green); middle panel: double staining for nTnG (red) and AQP-5 (purple); right panel: triple staining for nTnG, SOX17 and AQP-5 depicting alveolar AT1 cells (arrowheads) and endothelial cells (arrows); scale bar=20 μm. Images are representative of n=3 mice. FIG. 5I shows staining of a typical donor-derived patch for CD31 (green) and single molecule RNA FISH probe for SPC (cyan), demonstrating endothelial and AT2 cells; scale bar=20 μm. FIG. 5J-K shows graphical summary of quantitative differences between the composition of patches derived from transplantation of CD326+CD31+ and CD326-CD31+ cells. FIG. 5J is a graph of donor-derived nuclei/patch, showing that larger patches are derived from CD326+CD31+ compared to CD326-CD31+ cells; p=0.035, student's ttest; n=20 patches were evaluated from n=3 mice in each group. FIG. 5K is a graph of absolute number of donor-derived epithelial and endothelial cells per patch, after transplantation from CD326+CD31+ or CD326-CD31+ cells: A and B-depict epithelial cells derived from transplanted CD326-CD31+ and CD326+CD31+ populations respectively, p=0.0001, student's ttest; C and D depict endothelial cells derived from transplanted CD326-CD31+ and CD326+CD31+ populations respectively, p=0.04, student's ttest; n=10 patches from 2 mice were evaluated for each group.

FIGS. 6A-J demonstrate different transgenes expression in double positive CD326+CD31+ patch-forming lung cell progenitors. CD326+CD31+ lung cells of transgenic reporter mice expressing GFP under the Shh (FIGS. 6A, 6C, 6F), or VE-cadherin (FIGS. 6B, 6C, 6G) promoters were analyzed (n=6 mice), for the expression of these markers by FACS and by Imagestream analysis. Cells of these mice express td tomato and are red but express GFP and become green upon the expression of the transgene. FIG. 6A-B show typical dot plots demonstrating GFP expression of gated CD326+CD31+double positive cells. FIG. 6C shows percentage of CD326+CD31+double positive cells compared to CD326+VEcad+ cells in VE cadherin Cre mTmG or CD31+Shh+ cells in Shh Cre mTmG mice. The figure shows results of individual transgenic mice, n=6 for each genotype. FIG. 6D-Left shows culture of FACS purified CD326+VEcad⁻(mT) and CD326+VEcad+ (mG) lung cell populations from VEcad mTmG mice under 3D conditions (For FACS sorting scheme see FIG. 14A). The CD326+VEcad− cells not expressing VEcad lead to mT+ red organoids not expressing GFP, while double positive CD326+VEcad+ cells lead to generation of green organoids, expressing both GFP and different epithelial markers such as CK, AQP-5 and SPC (magenda). FIG. 6D-right shows absolute number of observed organoids per well upon seeding of 5×10⁵ CD326+VEcad− or CD326+VEcad+ FACS sorted lung cells. FIG. 6E Upper panel shows organoids exhibiting GFP (green, derived for VE-Cadhering expressing cells) and the epithelial marker cytokeratin (magenta). FIG. 6E Lower panel, left, shows organoids exhibiting GFP (green, derived from VE-Cadhering expressing cells) and the epithelial marker AQP-5 (Magenta) for alveolar AT1 lung cells. FIG. 6E Lower panel, right, shows organoids exhibiting GFP (green, derived from VE-Cadhering expressing cells) and the epithelial marker SPC (Magenta) for alveolar AT2 lung cells. FIGS. 6F-G show Imagestream analysis of CD326+CD31+ lung cell progenitors from Shh Cre nTnG mice or VEcad Cre nTnG mice, illustrating Shh and VE-cad expression in these cells. FIG. 6H-J show FACS analysis of double positive CD326+CD31+ lung cells from Nkx 2.1 Cre ER2 mTmG mice (n=3), Ager Cre ER2 mTmG mice (n=5) and Hopx Cre ER2 mTmG mice (n=3), defining the percentage of Nkx 2.1+, Ager+ and Hopx+ cells.

FIGS. 7A-E show staining of regenerative patches in the chimeric lung for epithelial and endothelial markers. FIG. 7A shows donor derived bronchial patch, demonstrating massive engraftment of the donor cells in the bronchus, which positively stain for membranous Ecad (bluc) and nuclear Nkx-2.1 (cyan), nulei (yellow), scale bar=20 μm. FIG. 7B shows donor derived bronchio-alveolar patch stained with anti CD31 (blue), and anti-cytokeratin (cyan) antibodies, demonstrating chimerism in epithelial and endothelial compartments, scale bar=50 μm. FIG. 7C shows staining of donor derived bronchial patch with anti-CD31 (blue) and anti CC-10 (cyan) antibodies and nuclei (grey), scale bar=50 μm, demonstrating engraftment in the compartment of secretory cells. The images shown are representative of n=3 mice. FIG. 7D shows staining with anti-AQP-5 (green) antibody demonstrating presence of donor derived mTom AT1 cells (red) within the patch, nuclei (blue), scale bar=5 μm. FIG. 7E shows staining of an alveolar patch with SPC probe (smFISH) (green) and anti-CD31 antibody (cyan) demonstrating presence of AT2 and endothelial cells within the patch, nuclei (grey), scale bar=10 μm.

FIGS. 8A-E demonstrate transplantation of adult R26R-Confetti BM from donors induced to express Cre-recombination by tamoxifen. FIG. 8A is a schematic presentation of the spleen colony assay using adult BM from R26R-Confetti donors. FIG. 8B-C show FACS analysis demonstrating expression of fluorescent Tags within LSK+ BM progenitors prior to BM transplantation. BM was analysed from n=8 adult R26R-Confetti mice, in two independent experiments. FIG. 8D shows formation of monochromatic spleen colonies 9 days after transplantation of BM from R26R-Confetti donors into lethally irradiated mice. FIG. 8E shows peripheral blood chimerism, demonstrating existence of fluorescent clones in the hematological compartment, originating from the transplanted Cr-recombined cells. The results shown in FIGS. 8D-E are representative of two independent experiments, n=7 mice in each experiment.

FIGS. 9A-E demonstrate transplantation of E16 R26R-Confetti liver into NA+6Gy preconditioned mice. FIG. 9A is the experimental workflow. FIG. 9B shows FACS analysis of E16 confetti fetal liver 4 days after Tmx administration, demonstrating expression of the 4 fluorescent tags within the Scal+Ckit+ hematopoietic progenitor cell population. FIG. 9C shows monochromatic spleen colonies generated by fetal liver cells transplanted into NA+6Gy TBI preconditioned mice. Scale bar=500 μm. FIG. 9D shows FACS analysis of peripheral blood from the chimeric mice 2 months after transplantation, demonstrating persistence of monochromatic clones in the hematological compartment. FIG. 9E shows a representative lung two photon image of a mouse preconditioned with Na+6GY TBI and transplanted with E16 fetal liver cells, demonstrating presence of isolated fluorescent cells and absence of monochromatic donor-derived patches, confirming the unique ability of lung cells to mediate lung regeneration. Scale bar=50 μm. The results are representative of n=10 mice, transplanted in two independent experiments.

FIGS. 10A-B demonstrate appearance of cleared chimeric lung sample prior to evaluation with LSM. Staining of cleared chimeric lungs is shown in FIGS. 3E-F.

FIGS. 11A-B demonstrate staining of lungs from chimeric mice transplanted with FACS sorted CD326+CD31+ cells derived from mTmG mice. FIG. 11A shows staining of the regenerative patch with anti-CD31 (bluc) and anti-Nkx 2.1 antibody (cyan), demonstrating presence of epithelial and endothelial cells within the regenerative patch, scale bar=50 μm. FIG. 11B shows staining of the sample shown in FIG. 11A with anti AQP-5 (cyan) and anti-CD31 antibody (blue), demonstrating presence of donor derived AT1 cells (indicated with arrows) and endothelial cells (indicated with arrow heads), in the engrafted area, scale bar=10 μm.

FIGS. 12A-D demonstrate long term chimerism at 9 months post-transplantation in mice transplanted with CD326+CD31+ nTnG FACS sorted cells. FIG. 12A shows whole mount lung tissue evaluated by confocal microscopy; scale bar=500 μm. FIG. 12B shows confocal images of the chimeric lung slice stained with anti-Lyve-1 (blue) and anti HOPX (green) antibodies, under low magnification, demonstrating large scale donor-derived cell engraftment in different compartments of the lung; scale bar=200 μm. FIG. 12C is a confocal image of the chimeric lung stained with anti-CD31 (blue), demonstrating endothelial cells with nT nuclei (indicated with arrow heads) and anti-SPC (green), demonstrating AT2 cells with nT nuclei (indicated with arrows); scale bar=20 μm. FIG. 12D shows staining of chimeric lung with anti-Hopx antibody, demonstrating donor derived AT1 cells (indicated with arrows); scale bar=20 μm.

FIGS. 13A-E demonstrate FACS analysis of lungs from transgenic mice. FIG. 13A shows typical dot plot depicting double positive mG-Shh+CD31+ cells in the lung of Shh Cre mTmG mouse; n=6. FIG. 13B shows typical dot plot depicting double positive CD326+mG-VEcad+ cells in the lung of VE cad Cre mTmG mouse; n=6. FIG. 13C shows typical dot plot depicting double positive mG-Nkx 2.1+CD31+ cells in the lung of Nkx 2. Cre ER2 mTmG mouse, n=5. FIG. 13D shows typical dot plot depicting double positive mG-Ager+CD31+ cells in the lung of Ager Cre ER2 mTmG lung, n=5. FIG. 13E shows typical dot plot depicting double positive mG-Hopx+CD31+ cells in the lung of Hopx Cre ER2 mTmG lung; n=3.

FIGS. 14A-D demonstrate staining for epithelial markers of organoids grown from FACS purified CD326+VEcad-harvested from lungs of VEcad mTmG mice. FIG. 14A shows the gating strategy for purification of single positive CD326+VE cad mG− and double positive CD326+VE cad mG+ cells from VEcad mTmG mice. Live CD45-TER119-Sytox-CD326+ single cells were further gated according to the expression of VE-cad mG, so as to purify CD326+VE-cad mG− and CD326+VE cad mG+ lung cell subpopulations. The isolated cells were used to generate lung organoids as described in Methods hereinbelow. The organoids were stained with anti-CK antibody (FIG. 14B) (cyan, scale bar=20 μm), anti-AQP-5 antibody (FIG. 14C), (cyan, scale bar=10 μm) and anti-SPC antibody (FIG. 14D) (cyan, scale bar=7 μm). Nuclei were stained by DAPI (blue). Images are representative of two independent experiments.

FIGS. 15A-C demonstrate that different culture media have different effects on expansion of pulmonary cells which dually express endothelial and epithelial markers. FIG. 15A Upper panel shows FACS analysis demonstrating levels of double positive CD326+CD31+ cells at different culture time points upon incubation in a conditioned medium (CM) obtained from mouse fibroblatss supplemented with EGF and a low concentration of ROCK inhibitor (5 μm) (marked as “original medium”). FIG. 15A Lower Pannel shows FACS analysis demonstrating levels of double positive CD326+CD31+ cells at different culture time points upon incubation in CM supplemented with EGF, VEGF and a high ROCK inhibitor concentration (20 μm) (marked as “CM+Epi+Endo+High RICK-I”). FIG. 15B shows total cells number at the indicated days in the two cultures described in FIG. 15A. FIG. 15C shows CD326+CD31+ double positive cells number at the indicated days of the two cultures described in FIG. 15A.

### DESCRIPTION OF SPECIFIC EMBODIMENTS OF THE INVENTION

The present invention, in some embodiments thereof, relates to pulmonary progenitor cells and, more particularly, but not exclusively, to methods of generating same and use of same in therapeutic applications.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Today, the only definitive treatment end stage respiratory diseases is by replacement of the damaged organ with a lung transplant. Due to a shortage of suitable organs, many patients die on the transplant waiting list, and therefore lung diseases are prime candidates for stem cell therapy.

Various cell populations have been shown to exhibit lung regenerative potential, including e.g. BM-derived or lung-derived cells, leading to marked lung host-donor chimerism. Notably, a large proportion of the donor derived patches exhibit different lineage lung compartments including epithelial and endothelial cells. The present inventors addressed the possibility that following transplantation of lung cells each donor patch originate from a single progenitor. To that end, as shown in the Examples section which follows (see Example 1), fetal or adult lung cells from Rosa26-Confetti mice bearing a multicolor Cre reporter system (9) were transplanted to recipients preconditioned with naphthalene and TBI. This four-color Cre recombination system provides a fetal or adult lung cell preparation in which each cell expresses just one randomly determined colour. Thus, the likelihood that each cell within a doublet in the transplanted cell population would be of the same colour is markedly reduced. Notably, immunohistochemistry, confocal microscopy, as well as two-photon microscopy and light sheet microscopy, demonstrated that all donor-derived lung patches developing after transplantation are monochromatic, strongly supporting the clonal origin of donor-derived lung patches observed after transplantation, in striking resemblance to the spleen colony forming cells typically identified after bone marrow transplantation. These results provided definitive evidence of a single multi-potential lung progenitor capable of differentiating into diverse lung cell lineages. In line with the observation that large proportion of the patches contain both endothelial and epithelial cells, and that each such a patch is derived from a single progenitor, the present inventors searched for a putative multipotent lung progenitor capable of differentiating along these two distinct lineages.

Following, while reducing embodiments of the present invention to practice, the present inventors have uncovered a novel population of pulmonary progenitor cells which dually express endothelial and epithelial markers (see Example 1 of the Examples section which follows). These pulmonary progenitor cells were obtained from both fetal and adult pulmonary tissues. Furthermore, these pulmonary progenitor cells were capable of differentiating into endothelial lung cells as well as into epithelial lung cells, and consequently were capable of generating endothelial and epithelial lung tissues following transplantation into a recipient. Taken together, these results substantiate the use of the novel pulmonary progenitor cells for e.g. regeneration of pulmonary organs or tissues, such as for the treatment of lung injury or disease.

Thus, according to an aspect of the present invention, there is provided a method of generating an isolated population of pulmonary cells, the method comprising:
(a) dissociating a pulmonary tissue so as to obtain a population of isolated pulmonary cells; and
(b) contacting said population of isolated pulmonary cells with at least one agent capable of binding an epithelial cell marker and an endothelial cell marker, so as to select a cell population being double positive for expression of epithelial and endothelial cell markers, thereby generating the isolated population of pulmonary cells.

Further, the present inventors found out that different culturing conditions have different effects on expansion of this novel pulmonary progenitor cells population. Specifically, it was shown that while culturing dissociated lung cells in a medium comprising VEGF, EGF and a ROCK inhibitor led to marked expansion of cells dually expressing endothelial and epithelial markers, culturing these cells in a medium comprising only EGF and a ROCK inhibitor resulted in expansion of the total number of cells but not of the double positive cells (Example 2 of the Examples section which follows). Taken together, these results substantiate the need for culturing lung cells under conditions allowing expansion of pulmonary progenitor cells which dually express endothelial and epithelial markers and/or qualifying expansion of these progenitor cells prior the use of the cultured cells for e.g. regeneration of pulmonary organs or tissues, such as for the treatment of lung injury or disease.

Thus, according to an aspect of the present invention, there is provided a method of expanding in culture an isolated population of pulmonary cells, the method comprising:
(a) dissociating a pulmonary tissue so as to obtain a population of isolated pulmonary cells; and
(b) expanding said population of isolated pulmonary cells in a medium comprising a factor that promotes expansion of endothelial cells, a factor that promotes expansion of epithelial cells and a factor that prevents differentiation, so as to expand a cell population being double positive for expression of epithelial and endothelial cell markers, thereby expanding in culture the isolated population of pulmonary cells.

According to an additional or an alternative aspect of the present invention, there is provided a method of qualifying suitability of an isolated population of pulmonary cells for administration to a subject in need thereof, the method comprising:
(a) dissociating a pulmonary tissue so as to obtain a population of isolated pulmonary cells;
(b) expanding said population of isolated pulmonary cells in a culture; and
(c) determining expression of epithelial and endothelial cell markers on said population of isolated pulmonary cells during and/or following said culture,
wherein expansion above a predetermined threshold of a cell population being double positive for expression of said epithelial and endothelial cell markers indicates said population of isolated pulmonary cells is suitable for administration to the subject; and
wherein no expansion or expansion below said predetermined threshold of said cell population being double positive for expression of said epithelial and endothelial cell markers indicates said population of isolated pulmonary cells is not suitable for administration to the subject,
thereby qualifying suitability of the isolated population of pulmonary cells for administration to the subject.

According to specific embodiments, the methods disclosed herein are affected ex-vivo.

The phrase “pulmonary tissue” as used herein refers to a lung tissue or organ. The pulmonary tissue of the present invention may be a full or partial organ or tissue. Thus, the pulmonary tissue of some embodiments may comprise the right lung, the left lung, or both. The pulmonary tissue of some embodiments of the invention may comprise one, two, three, four or five lobes (from either the right or the left lung). Moreover, the pulmonary tissue of some embodiments of the present invention may comprise one or more lung segments or lung lobules. Furthermore, the pulmonary tissue of some embodiments of the present invention may comprise any number of bronchi and bronchioles (e.g. bronchial tree) and any number of alveoli or alveolar sacs.

Depending on the application and available sources, the cells of the present invention may be obtained from a prenatal organism, postnatal organism, an adult or a cadaver donor. Such determinations are well within the ability of one of ordinary skill in the art.

It will be appreciated that the pulmonary cells of some embodiments of the invention may be of fresh or frozen (e.g., cryopreserved) preparations, as further discussed below.

According to specific embodiments, the pulmonary tissue is a human pulmonary tissue.

According to specific embodiments, the pulmonary tissue is from a cadaver donor.

According to specific embodiments, pulmonary tissue is from a living donor.

According to one embodiment, the pulmonary tissue is from an adult origin (e.g. a mammalian organism at any stage after birth).

According to one embodiment, the pulmonary tissue is from an embryonic origin.

According to one embodiment, the pulmonary tissue is from a fetal origin.

Accordingly, the embryonic or fetal organism may be of any of a human or xenogeneic origin (e.g. porcine) and at any stage of gestation. Such a determination is in the capacity of one of ordinary skill in the art.

Various methods may be employed to obtain an organ or tissue from an embryonic or fetal organism. Thus, for example, obtaining a pulmonary tissue may be effected by harvesting the tissue from a developing fetus, e.g. by a surgical procedure.

According to one embodiment, the pulmonary tissue (i.e. lung tissue) is obtained from a fetus at a stage of gestation corresponding to human canalicular stage of development (e.g. 16-25 weeks of gestation). According to one embodiment, the pulmonary tissue is obtained from a fetus at a stage of gestation corresponding to human 16-17 weeks of gestation, 16-18 weeks of gestation, 16-19 weeks of gestation, 16-20 weeks of gestation, 16-21 weeks of gestation, 16-22 weeks of gestation, 16-24 weeks of gestation, 17-18 weeks of gestation, 17-19 weeks of gestation, 17-20 weeks of gestation, 17-21 weeks of gestation, 17-22 weeks of gestation, 17-24 weeks of gestation, 18-19 weeks of gestation, 18-20 weeks of gestation, 18-21 weeks of gestation, 18-22 weeks of gestation, 18-24 weeks of gestation, 19-20 weeks of gestation, 19-21 weeks of gestation, 19-22 weeks of gestation, 19-23 weeks of gestation, 19-24 weeks of gestation, 20-21 weeks of gestation, 20-22 weeks of gestation, 20-23 weeks of gestation, 20-24 weeks of gestation, 21-22 weeks of gestation, 21-23 weeks of gestation, 21-24 weeks of gestation, 22-23 weeks of gestation, 22-24 weeks of gestation, 22-25 weeks of gestation, 23-24 weeks of gestation, 23-25 weeks of gestation, 24-25 weeks of gestation or 25-26 weeks of gestation.

According to a specific embodiment, the pulmonary tissue is obtained from a fetus at a stage of gestation corresponding to human 20-22 weeks of gestation.

According to a specific embodiment, the pulmonary tissue is obtained from a fetus at a stage of gestation corresponding to human 21-22 days of gestation.

According to a specific embodiment, the pulmonary tissue is obtained from a fetus at a stage of gestation corresponding to human 20-21 days of gestation.

It will be understood by those of skill in the art that the gestational stage of an organism is the time period elapsed following fertilization of the oocyte generating the organism. The following table provides an example of the gestational stages of human and porcine tissues at which these can provide fetal tissues which are essentially at corresponding developmental stages:

TABLE 1
Corresponding gestational stages of pigs and humans

| Gestational stage of porcine | Gestational stage of |
|---|---|
| pulmonary tissue (days) | human tissue (days**) |
| 18 | 44 |
| 20 | 49 |
| 22 | 54 |
| 23 | 56-57 |
| 25 | 61-62 |
| 26 | 63 |
| 28 | 68-69 |
| 31 | 75 |
| 38 | 92 |
| 42 | 102 |
| 46 | 112 |
| 49 | 119 |
| 56 | 136 |
| 62 | 151 |
| 72 | 175 |
| 80 | 195 |
| 88 | 214 |

*The gestational stage (in days) of a tissue belonging to a given species which is at a developmental stage essentially corresponding to that of a porcine tissue can be calculated according to the following formula: [gestational stage of porcine tissue in days]/[gestational period of pig in days] × [gestational stage of tissue of given species in days]. Similarly, the gestational stage (in days) of a tissue belonging to a given species which is at a developmental stage essentially corresponding to that of a human tissue can be calculated according to the following formula: [gestational stage of human tissue in days]/[gestational period of humans in days] × [gestational stage of tissue of given species in days]. The gestational stage of pigs is about 115 days and that of humans is about 280 days.
**for week calculation divide the numbers by 7.

Likewise, various methods may be employed to obtain a pulmonary organ or tissue from an adult organism (e.g. live or cadaver). Thus, for example, obtaining a pulmonary tissue may be effected by harvesting the tissue from an organ donor by a surgical procedure e.g. laparotomy or laparoscopy. After the organ/tissue is obtained from the adult organism, pulmonary cells as well as hematopoietic progenitor cells (as discussed in detail below) may be isolated therefrom according to methods known in the art, such methods depend on the source and lineage of the cells and may include, for example, flow cytometry and cell sorting as taught for example by www (dot) bio-rad (dot) com/en-uk/applications-technologies/isolation-maintenance-stem-cells.

It will be appreciated that in order to obtain pulmonary cells, the pulmonary tissue need not be intact (i.e. maintain a tissue structure such that is suitable for a whole organ transplantation), however, the pulmonary tissue should comprise viable cells.

In addition, according to specific embodiments, the pulmonary tissue may be obtained from more than one donor. Thus, according to specific embodiments, the pulmonary cells may comprise cells obtained from more than one cell donor.

After a pulmonary organ/tissue is obtained (e.g. fetal or adult tissue), the present invention further contemplates generation of an isolated population of cells therefrom. The phrase “isolated population of pulmonary cells” refers to isolated cells which do not form a tissue structure (i.e., no connective tissue structure).

Thus, the pulmonary cells may be comprised in a suspension of single cells or cell aggregates of no more than 5, 10, 50, 100, 200, 300, 400, 500, 1000, 1500, 2000 cells in an aggregate.

The phrase “pulmonary cells in suspension” as used herein refers to cells which have been isolated from their natural environment (e.g., the human body) are extracted from the pulmonary tissue while maintaining viability but do not maintain a tissue structure (i.e., no vascularized tissue structure) and are not attached to a solid support.

The cell suspension of the invention may be obtained by any mechanical or chemical (e.g. enzymatic) means. Several methods exist for dissociating cell clusters to form cell suspensions (e.g. single cell suspension) from primary tissues, attached cells in culture, and aggregates, e.g., physical forces (mechanical dissociation such as cell scraper, trituration through a narrow bore pipette, fine needle aspiration, vortex disaggregation and forced filtration through a fine nylon or stainless steel mesh), enzymes (enzymatic dissociation such as trypsin, collagenase, Accutase and the like) or a combination of both.

According to specific embodiments, the dissociating is by enzymatic digestion.

Thus, for example, enzymatic digestion of tissue/organ into isolate cells can be performed by subjecting the tissue to an enzyme such as type IV Collagenase (Worthington biochemical corporation, Lakewood, NJ, USA) and/or Dispase (Invitrogen Corporation products, Grand Island NY, USA). For example, the tissue may be enzyme digested by finely mincing tissue with a razor blade in the presence of e.g. collagenase, dispase and CaCl₂) at 37° C. for about 1 hour. The method may further comprise removal of nonspecific debris from the resultant cell suspension by, for example, sequential filtration through filters (e.g. 70- and 40-μm filters), essentially as described in the Examples section which follows.

Furthermore, mechanical dissociation of tissue into isolated cells can be performed using a device designed to break the tissue to a predetermined size. Such a device can be obtained from CellArtis Goteborg, Sweden. Additionally or alternatively, mechanical dissociation can be manually performed using a needle such as a 27g needle (BD Microlance, Drogheda, Ireland) while viewing the tissue/cells under an inverted microscope.

Following enzymatic or mechanical dissociation of the tissue, the dissociated cells are further broken to small clumps using e.g. 200 μl Gilson pipette tips (e.g., by pipetting up and down the cells).

According to specific embodiments, the cell suspension of pulmonary cells comprises viable cells. Cell viability may be monitored using any method known in the art, as for example, using a cell viability assay (e.g. MultiTox Multiplex Assay available from Promega), Flow cytometry, Trypan blue, etc.

According to the teachings of the present invention, the pulmonary tissue and isolated cells derived therefrom comprise cells that express both epithelial and endothelial cell markers.

According to specific embodiments, these cells expressing both epithelial and endothelial cell markers are progenitor cells capable of differentiating into both endothelial lung cells and epithelial lung cells. Methods of determining differentiation include in-vitro and in-vivo (e.g. transplantation) methods well known to the skilled in the art. Non-limiting examples are provided in the Examples section which follows.

Hence, according to one embodiment, the pulmonary cells are characterized by the expression of epithelial and endothelial cell markers.

The phrase “epithelial cell marker” as used herein refers to a cell-surface protein characteristic of lung epithelial cells. Such a marker includes, but is not limited to, CD326, CD324, CD24, Aquaporin 5 (AQP-5), Podoplanin (PDPN), and Advanced Glycosylation End-Product Specific Receptor (RAGE, i.e. encoded by AGER gene).

The phrase “endothelial cell marker” as used herein refers to a cell-surface protein characteristic of lung endothelial cells. Such a marker includes, but is not limited to, CD31 and CD144 (VE-cadherin).

According to a specific embodiment, the pulmonary cell are characterized by the co-expression signature: CD326⁺ and CD31⁺.

According to a specific embodiment, the pulmonary cell are characterized by the co-expression signature: CD324⁺ and CD31⁺.

According to a specific embodiment, the pulmonary cell are characterized by the co-expression signature: CD24⁺ and CD31⁺.

According to a specific embodiment, the pulmonary cell are characterized by the co-expression signature: AQP-5⁺ and CD31⁺.

According to a specific embodiment, the pulmonary cell are characterized by the co-expression signature: PDPN⁺ and CD31⁺.

According to a specific embodiment, the pulmonary cell are characterized by the co-expression signature: RAGE⁺ and CD31⁺.

According to a specific embodiment, the pulmonary cell are characterized by the co-expression signature: CD326⁺ and CD144⁺.

According to a specific embodiment, the pulmonary cell are characterized by the co-expression signature: CD324⁺ and CD144⁺.

According to a specific embodiment, the pulmonary cell are characterized by the co-expression signature: CD24⁺ and CD144⁺.

According to a specific embodiment, the pulmonary cell are characterized by the co-expression signature: AQP-5⁺ and CD144⁺.

According to a specific embodiment, the pulmonary cell are characterized by the co-expression signature: PDPN⁺ and CD144⁺.

According to a specific embodiment, the pulmonary cell are characterized by the co-expression signature: RAGE⁺ and CD144⁺.

According to specific embodiments, the pulmonary cells are further characterized by expression of at least one of Nkx 2.1, CD200, Akap5, Sec1413, Prdx6 and Clic3.

According to specific embodiments, the pulmonary cells comprise a heterogeneous population of cells (e.g. unseparated population of cells) comprising the cells co-expressing the endothelial and epithelial marks.

According to other specific embodiments, the pulmonary cells comprise a purified population of cells. Accordingly, the cells may be treated to remove specific population of cells therefrom (e.g. removal of a subpopulation) or to positively select a desired population (e.g. a cell population being double positive for expression of epithelial and endothelial cell markers). Purification of specific cell types may be carried out by any method known to one of skill in the art, such as for example, eradication (e.g. killing) with specific antibodies or by affinity based purification (e.g. such as by the use of MACS beads, FACS sorter and/or capture ELISA labeling) using specific antibodies which recognize any specific cell markers (e.g. CD31, CD34, CD41, CD45, CD8, CD8, CD48, CD105, CD150, CD271, CD326, MUCIN-1, PODOPLANIN etc.). Such methods are described herein and in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 1992). For example, cells can be sorted by, for example, flow cytometry or FACS. Thus, fluorescence activated cell sorting (FACS) may be used and may have varying degrees of color channels, low angle and obtuse light scattering detecting channels, and impedance channels. Any ligand-dependent separation techniques known in the art may be used in conjunction with both positive and negative separation techniques that rely on the physical properties of the cells rather than antibody affinity, including but not limited to elutriation and density gradient centrifugation. Other methods for cell sorting include, for example, panning and separation using affinity techniques, including those techniques using solid supports such as plates, beads and columns. Thus, biological samples may be separated by “panning” with an antibody attached to a solid matrix, e.g. to a plate. Alternatively, cells may be sorted/separated by magnetic separation techniques, and some of these methods utilize magnetic beads. Different magnetic beads are available from a number of sources, including for example, Dynal (Norway), Advanced Magnetics (Cambridge, MA, U.S.A.), Immuncon (Philadelphia, U.S.A.), Immunotec (Marseille, France), Invitrogen, Stem cell Technologies (U.S.A) and Cellpro (U.S.A). Alternatively, antibodies can be biotinylated or conjugated with digoxigenin and used in conjunction with avidin or anti-digoxigenin coated affinity columns.

According to an embodiment, different depletion/separation methods can be combined, for example, magnetic cell sorting can be combined with FACS, to increase the separation quality or to allow sorting by multiple parameters.

According to specific embodiments, such a selection is effected by contacting with an agent capable of binding the desired marker(s). Such an agent my be an antibody, for example. The antibody may be monospecific or at least bispecific.

According to a specific embodiment, the pulmonary cells comprise a purified population of cells expressing both epithelial and endothelial markers.

According to specific embodiments, the selection is effected by contacting the population of isolated pulmonary cells with at least one agent capable of binding an epithelial cell marker and an endothelial cell marker, and selecting a cell population being double positive for expression of the epithelial and endothelial cell markers.

According to specific embodiments, the at least one agent is a single agent. In such a case the agent has specificity to both the endothelial marker and the epithelial marker.

According to specific embodiments, the at least one agent comprises at least two agents. In such a case at least one of the agents has specificity to the endothelial marker and the at least one of the agent has specificity to the epithelial marker.

According to specific embodiments, the at least one agent is an antibody.

According to specific embodiments, the at least one antibody is a monospecific antibody. In this case contacting is effected with two distinct antibodies, one having specificity to the endothelial marker and the other having specificity to the epithelial marker.

According to specific embodiments the antibody is a bispecific antibody. In this case contacting may be effected with one antibody having specificity for both the endothelial and the epithelial markers.

According to specific embodiments, at least about 0.1%, 1%, 2%, 5%, 10%, 20%, 30%, 40%, 50%, 60%, 70%, 80%, 90% or 95% of the pulmonary cells generated by the method of some embodiments of the invention are characterized by the dual expression of epithelial and endothelial cell markers (e.g. are CD326⁺CD31⁺, CD324⁺CD31⁺, CD326⁺CD144⁺, or CD324⁺CD144⁺).

According to specific embodiments, about 0.1-10%, 0.1-20%, 0.1-50%, 0.1-100%, 1-10%, 1-20%, 1-50%, 1-100%, 10-20%, 10-50%, 10-100%, e.g. about 20-30%, e.g. about 20-40%, e.g. about 20-60%, e.g. about 30-50%, e.g. about 30-70%, e.g. about 40-50%, e.g. about 40-80%, e.g. about 50-60%, e.g. about 50-70%, e.g. about 60-80%, e.g. about 60-90%, e.g. about 70-90%, e.g. about 80-100% of the pulmonary cells generated by the method of some embodiments of the invention are characterized by the dual expression of epithelial and endothelial cell markers (e.g. are CD326⁺CD31⁺, CD324⁺CD31⁺, CD326⁺CD144⁺, or CD324⁺CD144⁺).

According to specific embodiments, at least about 0.1%, 1%, 2%, 5% or 10% of the pulmonary cells generated by the method of some embodiments of the invention are characterized by the dual expression of epithelial and endothelial cell markers.

According to specific embodiments, about 0.1-10% or 1-10% of the pulmonary cells generated by the method of some embodiments of the invention are characterized by the dual expression of epithelial and endothelial cell markers.

According to specific embodiments, at least 20%, 30%, 40% or 50% of the pulmonary cells generated by the method of some embodiments of the invention are characterized by the dual expression of epithelial and endothelial cell markers.

Also provided herein is a kit for isolation of pulmonary cells characterized as being double positive for expression of epithelial and endothelial cell markers, the kit comprising at least one agent capable of binding:
(i) CD31 or CD144; and
(ii) CD326, CD324, CD24, Aquaporin 5 (AQP-5), Podoplanin (PDPN), or Advanced Glycosylation End-Product Specific Receptor (RAGE).

According to specific embodiments, the kit further comprises instructions for use.

According to specific embodiments, the selection is effected prior to culturing.

According to specific embodiments, the selection is effected following or during culturing.

According to specific embodiments, the selection is effected prior to administration of the cells to a subject in need thereof.

According to one embodiment, the pulmonary cells are characterized by the lack of expression of leukocyte cell markers.

According to a specific embodiment, pulmonary cells are characterized by the lack of expression of CD45.

Thus, according to specific embodiments, the method comprises depleting CD45 expressing cells.

Methods of depleting cells are well known to the skilled in the art and ae further described hereinabove and below. According to specific embodiments, depleting CD45 expressing cells is affected by contacting the population of isolated pulmonary cells with an agent capable of binding CD45, so as to select a cell population being negative for expression of CD45.

According to an embodiment, the pulmonary cells comprise less than 10%, less than 50% or less that 2% CD45+ cells.

According to one embodiment, the pulmonary cells are depleted of T cells.

Thus, according to specific embodiments, the method comprises depleting T cells.

Methods of depleting T cells are well known to the skilled in the art and ae further described hereinabove and below. According to specific embodiments, depleting T cells expressing cells is affected by contacting the population of isolated pulmonary cells with an agent capable of binding T cells, so as to select a cell population being negative for T cells.

As used herein the phrase “depleted of T cells” refers to a population of pulmonary cells which are depleted of T lymphocytes. The T cell depleted pulmonary cells may be depleted of CD3⁺ cells, CD2⁺ cells, CD8⁺ cells, CD4⁺ cells, a/B T cells and/or y/8 T cells.

According to an embodiment, the T cell depleted pulmonary cells comprise less than 10%, less than 50% or less that 2% T cells.

According to a specific embodiment, pulmonary cells are characterized by the lack of expression of CD3.

According to an embodiment, the therapeutically effective amount of T cell depleted pulmonary cells comprises less than 50×10⁵ CD3⁺ T cells, 40×10⁵ CD3⁺ T cells, 30×10⁵ CD3+ T cells, 20×10⁵ CD3⁺ T cells, 15×10⁵ CD3⁺ T cells, 10×10⁵ CD3⁺ T cells, 9×10⁵ CD3⁺ T cells, 8×10⁵ CD3⁺ T cells, 7×10⁵ CD3⁺ T cells, 6×10⁵ CD3⁺ T cells, 5×10⁵ CD3⁺ T cells, 4×10⁵ CD3⁺ T cells, 3×10⁵ CD3⁺ T cells, 2×10⁵ CD3⁺ T cells, 1×10⁵ CD3⁺ T cells or 5×10⁴ CD3⁺ T cells per kilogram body weight of the subject.

According to a specific embodiment, the pulmonary cells are characterized by the lack of expression of CD2.

According to a specific embodiment, the pulmonary cells are characterized by the lack of expression of CD4.

According to a specific embodiment, the pulmonary cells are characterized by the lack of expression of CD8.

According to an embodiment, the therapeutically effective amount of T cell depleted pulmonary cells comprises less than 50×10⁵ CD8⁺ cells, 25×10⁵ CD8⁺ cells, 15×10⁵ CD8⁺ cells, 10×10⁵ CD8⁺ cells, 9×10⁵ CD8⁺ cells, 8×10⁵ CD8⁺ cells, 7×10⁵ CD8⁺ cells, 6×10⁵ CD8⁺ cells, 5×10⁵ CD8⁺ cells, 4×10⁵ CD8⁺ cells, 3×10⁵ CD8⁺ cells, 2×10⁵ CD8⁺ cells, 1×10⁵ CD8⁺ cells, 9×10⁴ CD8⁺ cells, 8×10⁴ CD8⁺ cells, 7×10⁴ CD8⁺ cells, 6×10⁴ CD8⁺ cells, 5×10⁴ CD8⁺ cells, 4×10⁴ CD8⁺ cells, 3×10⁴ CD8⁺ cells, 2×10⁴ CD8⁺ cells or 1×10⁴ CD8⁺ cells per kilogram body weight of the subject.

According to a specific embodiment, the pulmonary cells are characterized by the lack of expression of a and B T cell receptor chains.

According to a specific embodiment, the pulmonary cells are characterized by the lack of expression of y and & T cell receptor chains.

According to one embodiment, the T cell depleted pulmonary cells are obtained by T cell debulking (TCD). T cell debulking may be effected using antibodies, including e.g. anti-CD8 antibodies, anti-CD4 antibodies, anti-CD3 antibodies, anti-CD2 antibodies, anti-TCRα/β antibodies and/or anti-TCRγ/δ antibodies.

According to one embodiment, the pulmonary cells are depleted of B cells.

According to an embodiment, the B cell depleted pulmonary cells comprise less than 10%, less than 50% or less that 2% B cells.

According to an embodiment, the therapeutically effective amount of pulmonary cells comprises less than 50×10⁵ B cells, 40×10⁵ B cells, 30×10⁵ B cells, 20×10⁵ B cells, 10×10⁵ B cells, 9×10⁵ B cells, 8×10⁵ B cells, 7×10⁵ B cells, 6×10⁵ B cells, 5×10⁵ B cells, 4×10⁵ B cells, 3×10⁵ B cells, 2×10⁵ B cells or 1×10⁵ B cells per kilogram body weight of the subject.

According to one embodiment, depletion of B cells is effected by B cell debulking. B cell debulking may be effected using antibodies, including e.g. anti-CD19 or anti-CD20 antibodies. Alternatively, debulking in-vivo of B cells can be attained by infusion of anti-CD20 antibodies.

T cell or B cell debulking may be effected in-vitro or in-vivo (e.g. in a donor prior to acquiring pulmonary tissue therefrom).

According to specific embodiments, the pulmonary cells comprise a heterogenous population of cells comprising, in addition to the cells co-expressing endothelial and epithelial markers, hematopoietic progenitor or precursor cells (HPCs), mesenchymal progenitor cells, epithelial cells, endothelial cells etc.

According to specific embodiments, the pulmonary cells are immediately used for transplantation.

According to other specific embodiments, the pulmonary cells are cultured ex-vivo.

As used herein, the term “culturing” or “culture” refers to at least pulmonary cells and culture medium in an ex-vivo environment. The culture is maintained under conditions capable of at least supporting viability of the pulmonary cells. Such conditions include for example an appropriate temperature (e.g., 37° C.), atmosphere (e.g., % O₂, % CO₂), pressure, pH, light, medium, supplements and the like.

The culture medium of some embodiments can be a water-based medium which includes a combination of substances such as salts, nutrients, minerals, vitamins, amino acids, antibiotics nucleic acids, proteins such as cytokines, growth factors and hormones, all of which are needed for maintaining the pulmonary cells in an viable state. For example, a culture medium can be a synthetic tissue culture medium such as RPMI-1640 (Life Technologies, Israel), Ko-DMEM (Gibco-Invitrogen Corporation products, Grand Island, NY, USA), DMEM/F12 (Biological Industries, Beit Hacmek, Israel), Mab ADCB medium (HyClone, Utah, USA), DMEM/F12 (Biological Industries, Biet Haemek, Israel), conditioned medium (e.g. from a feeder medium e.g. iMEF) supplemented with the necessary additives. Preferably, all ingredients included in the culture medium of the present invention are substantially pure, with a tissue culture grade.

According to specific embodiments, the medium is a conditioned medium.

A “conditioned medium (CM)” refers to a culture medium supplemented with a soluble factor (culture-derived growth factor) which was produced and secreted from cells (e.g. fibroblasts e.g. iMEF) cultured in the medium. As will be appreciated, the conditioned medium is substantially free of cells. Techniques for isolating conditioned media from a cell culture are well known in the art. Conditioned medium can also be commercially obtained from e.g. R&D Systems (e.g. MEF conditioned media, Cat no. AR005).

The culture may be in a glass, plastic or metal vessel that can provide an aseptic environment for tissue culturing. According to specific embodiments, the culture vessel includes dishes, plates, flasks, bottles and vials. Culture vessels such as COSTAR®, NUNC® and FALCON® are commercially available from various manufacturers.

According to specific embodiments, the culture vessel is a tissue culture plate.

According to specific embodiments, the culture is maintained under sterile conditions.

According to specific embodiments, the culture is maintained at 37-38° C.

According to specific embodiments, the pulmonary cells are cultured under conditions allowing their expansion. In other word, according specific embodiments, the pulmonary cells are expanded ex-vivo in a culture.

The term “expanding”, “expanded” or “proliferation” refers to an increase in the number of cells in a population by means of cell division. Methods of evaluating expansion are well known in the art and include, but not limited to, proliferation assays such as CFSE and BrDU and determining cell number by direct cell counting and microscopic evaluation.

According to specific embodiments, the expansion is by at least about 1.5 fold, at least about 2 fold, at least about 3 fold, at least about 4 fold, at least about 5 fold, at least about 10 fold, at least about 20 fold, at least about 40 fold, at least about 80 fold, at least about 120 fold, at least about 140 fold or more over a given time interval (and as compared to non-expanded cells e.g. prior to culturing).

According to specific embodiments, the pulmonary cells are cultured ex-vivo so as to expand the cell population being double positive for expression of epithelial and endothelial cell markers described herein.

According to specific embodiments, such conditions comprise a culture medium comprising a factor that promotes expansion of endothelial cells, a factor that promotes expansion of epithelial cells and a factor that prevents differentiation.

Endothelial cells are thin, flattened cells lining the interior surfaces of blood and lymphatic vessels, making up the endothelium. Herein, the term “endothelial cells” refers to isolated endothelial cells at any developmental stage, from progenitor to mature differentiated cells. The endothelial cells may express markers typical of the endothelial lineage including without limitation, CD31, CD144 (VE-Cadeherin), CD54 (I-CAM1), vWF, VCAM, CD106 (V-CAM), VEGF-R2.

Epithelial cells are cells lining any of the cavities or surfaces of structures throughout the mammalian body, making up the epithelium. The basic cells types are squamous, cuboidal, and columnar, classed by their shape. Herein, the term “epithelial cells” refer to isolated epithelial cells at any developmental stage, from progenitors to mature differentiated cells. The epithelial cells may express marks typical of the epithelial lineage including cytokeratin, CD326, CD324, CD24, Aquaporin 5 (AQP-5), Podoplanin (PDPN), Advanced Glycosylation End-Product, HOPX, Cytokeratin, Nkx 2.1, SP-A, SP-B, SP-D, Clara Cell Protein (CC16, CC10), Mucin-associated Antigens: KL-6, 17-Q2, 17-B1.

As used herein, “a factor that promotes expansion” refers to a biomolecule e.g., amino acid-based or nucleic acid-based or a small molecule chemical which promotes expansion in culture.

Factors that promote expansion of endothelial cells are well known in the art. Non-limiting examples which can be used with specific embodiments of the invention include vascular endothelial growth factor (VEGF), b-FGF, FGF2, IL-8, BMP4.

According to a specific embodiments, the factor that promotes expansion of endothelial cells comprises VEGF.

Non-limiting Examples of VEGF that can be used with specific embodiments of the invention include hVEGF 165, rh VEGF-121, rh VEGF-164, VEGF-c.

VEGF is commerically available from many vendors including e.g. Stemcell, R&D systems, Peprotech. According to specific embodiments, the VEGF is comprised in a medium such as an Endo medium which is commercially available from e.g. Sartorius.

According to some embodiments of the invention, the factor that promotes expansion of endothelial cells (e.g. VEGF) is provided at a concentration of at least 0.1 ng/ml, at least 0.5 ng/ml, at least 1 ng/ml, at least 5 ng/ml, or at least 10 ng/ml.

According to specific embodiments, the factor that promotes expansion of endothelial cells (e.g. VEGF) is provided at a concentration of no more than 10 μg/ml, no more than 1 μg/ml, no more than 100 ng/ml.

According to specific embodiments, the factor that promotes expansion of endothelial cells (e.g. VEGF) is provided at a concentration of 5-100 ng/ml. According to specific embodiments, the factor that promotes expansion of endothelial cells (e.g. VEGF) is provided at a concentration of about 30 ng/ml.

Factors that promote expansion of epithelial cells are well known in the art. Non-limiting examples which can be used with specific embodiments of the invention include epidermal growth factor (EGF), Noggin, R-Spondin.

According to a specific embodiment, the factor that promotes expansion of epithelial cells comprises EGF (e.g. hEGF).

EGF is commerically available from many vendors including e.g. Stemcell, R&D systems, Sigma-Aldrich.

According to some embodiments of the invention, the factor that promotes expansion of epithelial cells (e.g. EGF) is provided at a concentration of at least 0.1 ng/ml, at least 0.5 ng/ml, at least 1 ng/ml, at least 5 ng/ml, or at least 10 ng/ml.

According to specific embodiments, the factor that promotes expansion of epithelial cells (e.g. EGF) is provided at a concentration of no more than 10 μg/ml, no more than 1 μg/ml, no more than 100 ng/ml.

According to specific embodiments, the factor that promotes expansion of epithelial cells (e.g. EGF) is provided at a concentration of 5-100 ng/ml.

According to specific embodiments, the factor that promotes expansion of epithelial cells (e.g. EGF) is provided at a concentration of about 30 ng/ml.

As used herein “a factor that prevents differentiation” refers to a biomolecule e.g., amino acid-based or nucleic acid-based or a small molecule chemical which, alone or in combination with other factors, prevents differentiation of progenitor cells in culture (i.e. maintains their pluripotent state).

Factors that prevent differentiation are well known in the art. Non-limiting examples which can be used with specific embodiments of the invention include a ROCK inhibitor, a GSK3b inhibitor (e.g., CHIR99021), an ALK5 inhibitor (e.g. A83-01).

According to a specific embodiment, the factor that prevents differentiation comprises a ROCK inhibitor.

Many ROCK inhibitors are known in the art and are commerically available. Non-limiting examples include Y27632 (TOCRIS, Catalogue number 1254), Blebbistatin (TOCRIS Catalogue number 1760) and Thiazovivin (Axon Medchem-Axon 1535).

According to some embodiments of the invention, the factor that prevents differentiation (e.g. ROCK inhibitor) is provided at a concentration of at least 0.1 μM, at least 0.5 μM, at least 1 μM, at least 5 μM, or at least 10 μM.

According to specific embodiments, the factor that prevents differentiation (e.g. ROCK inhibitor) is provided at a concentration of no more than 10 mM, no more than 1 mM, no more than 100 μM.

According to specific embodiments, the factor that prevents differentiation (e.g. ROCK inhibitor) is provided at a concentration of 5-50 μM.

According to specific embodiments, the factor that prevents differentiation (e.g. ROCK inhibitor) is provided at a concentration of about 20 M.

According to specific embodiments, culturing is effected until a desired number of viable cells is obtained. Measuring the number of cells (e.g. viable cells) can be carried out using any method known to one of skill in the art, e.g. by a counting chamber, by FACs analysis, or by a spectrophotometer.

According to specific embodiments, the culture or the expansion is effected for at least 12 hours, for at least 24 hours, for at least 36 hours, for at least 48 hours, for at least 72 hours.

According to specific embodiments, the culture or the expansion is effected for at least 4 days, at least 5 days, at least 6 days, at least 7 days, at least 10 days, at least 14 days, at least 21 days, at least 24 days.

According to specific embodiments, the culture or the expansion is effected for 20-30 days.

According to specific embodiments, the culture or the expansion is effected for up to 5 weeks or up to 4 weeks.

According to specific embodiments, the culture or the expansion is effected is effected until reaching a cell number of at least 1×10⁷ cells.

According to specific embodiments, the culture or the expansion is effected is effected until reaching a cell number of at least 50,000, at least 100,000, at least 150,000, at least 200,000 cells expressing both epithelial and endothelial markers.

According to specific embodiments, the method further comprises determining expression of epithelial and endothelial cell markers on the pulmonary cells.

According to specific embodiments, the determining is effected following the selection.

According to specific embodiments, the determining is effected prior to the culture.

According to specific embodiments, the determining is effected during and/or following the culture.

Methods of determining expression are well known in the art and include flow cytometry, immunocytochemistry, western blot, PCR and the like.

According to specific embodiments, determining expression is effected by flow cytometry.

According to specific embodiments, expansion above a predetermined threshold of a cell population being double positive for expression of the epithelial and endothelial cell markers indicates the population of isolated pulmonary cells is suitable for administration to the subject. On the other hand, according to specific embodiments, no expansion or expansion below a predetermined threshold of a cell population being double positive for expression of the epithelial and endothelial cell markers indicates the population of isolated pulmonary cells is not suitable for administration to the subject. Following, according to specific embodiments, when no expansion or expansion below the predetermined threshold is detected the cells are either cultured again until expansion of the double positive cells is obtained or discarded.

According to specific embodiments, such a predetermined threshold is determined in comparison to the total number of the epithelial/endothelial double positive cells per se.

Thus, according to specific embodiments, expansion above a predetermined threshold is an increase of at least about 1.5 fold, at least about 2 fold, at least about 3 fold, at least about 4 fold, at least about 5 fold, at least about 10 fold or more in the number of the epithelial/endothelial double positive cells as compared to their number prior to the culturing.

According to specific embodiments, expansion above a predetermined threshold is an increase of at least 5%, at least 10%, at least 15%, at least 20%, at least 30%, at least 40%, at least 50%, at least 60%, at least 70%, at least 80%, at least 90% at least 100% or more in the number of the epithelial/endothelial double positive cells as compared to their number prior to the culturing.

According to specific embodiments, expansion below a predetermined threshold is an increase of less than about 1.5 fold, less than about 2 fold, less than about 3 fold, less than about 4 fold, less than about 5 fold, less than about 10 fold in the number of the epithelial/endothelial double positive cells as compared to their number prior to the culturing.

According to specific embodiments, expansion below a predetermined threshold is an increase of less than 5%, less than 10%, less than 15%, less than 20%, less than 30%, less than 40%, less than 50%, less than 60%, less than 70%, less than 80%, less than 90% or less than 100% in the number of the epithelial/endothelial double positive cells as compared to their number prior to the culturing.

According to specific embodiments, the determining is effected following at least 12 hours, at least 24 hours, at least 36 hours, at least 48 hours, or at least 72 hours of culture.

According to specific embodiments, the determining is effected following at least 4 days, at least 5 days, at least 6 days, at least 7 days, at least 10 days, at least 14 days, at least 21 days or at least 24 days of culture.

According to specific embodiments, the determining is effected following 20-30 days of culture.

According to specific embodiments, the determining is effected following up to 5 weeks or up to 4 weeks of culture.

The pulmonary tissue or cells derived therefrom of some embodiments of the invention may be stored under appropriate conditions (typically by freezing) at any step (e.g. following dissociation, following selection, prior to culturing, during culturing, following culturing) to keep the cells alive and functioning for use in transplantation. According to one embodiment, the pulmonary cells are stored as cryo-preserved populations. Other preservation methods are described in U.S. Pat. Nos. 5,656,498, 5,004,681, 5,192,553, 5,955,257, and 6,461,645. Methods for banking stem cells are described, for example, in U.S. Patent Application Publication No. 2003/0215942.

Thus, according to an aspect of the present invention, there is provided a cell bank comprising:
(i) a plurality of isolated populations of pulmonary cells in suspension, said pulmonary cells being characterized as double positive for the expression of epithelial and endothelial cell markers, and wherein said plurality of said isolated populations of said pulmonary cells have been HLA typed to form an allogeneic cell bank, each individually disposed within separate containers; and
(ii) a catalogue which comprises information about said HLA typed cells of said plurality of said isolated populations of said pulmonary cells.

The present invention, in some embodiments thereof, also contemplates cells obtainable or obtained by the methods disclosed herein.

Thus, according to an aspect of the present invention, there is provided an isolated population of pulmonary cells obtained according to the method.

According to specific embodiments, the isolated population of pulmonary cells comprises at least 10%, at least 15%, at least 20%, at least 25%, at least 30%, at least 35%, at least 40% cells expression both epithelial and endothelial markers e.g. CD326⁺CD31⁺ cells.

According to an aspect of the present invention, there is provided an isolated population of pulmonary cells comprising at least 40% CD326⁺CD31⁺ cells.

According to specific embodiments, the isolated population of pulmonary cells comprising at least 50%, at least 60%, at least 70%, at least 80% CD326⁺CD31⁺ cells.

According to specific embodiments, the pulmonary cells disclosed herein are capable of regenerating an epithelial pulmonary tissue.

According to specific embodiments, the pulmonary cells disclosed herein are capable of regenerating an endothelial pulmonary tissue.

According to specific embodiments, the cells can be grown in 2D or 3D cultures.

According to specific embodiments, the cells are in suspension.

According to specific embodiments, the cells are embedded or attached to a scaffold or a carrier which allows growth in suspension.

Scaffold material may comprise natural (e.g. fibrinogen, fibrin, thrombin, chitosan, collagen, alginate, poly(N-isopropylacrylamide), albumin, collagen, synthetic polyamino acids, prolamines, polysaccharides such as alginate, heparin, and other naturally occurring biodegradable polymers of sugar units) or synthetic organic polymers (e.g. such as PLGA, PMMA and PCL), that can be gelled, or polymerized or solidified (e.g., by aggregation, coagulation, hydrophobic interactions, or cross-linking) into a two-dimensional or a three-dimensional structure. Such scaffolds are known in the art and disclosed e.g. in Florian Weinberger et al. (2017) Circulation Research. 120:1487-1500; Rochkind S et al (2004) Neurol Res. 26 (2): 161-6; Rochkind S. et al. (2006) Eur Spine J. 15 (2): 234-45; FSY Wong, ACY Lo (2015) J Stem Cell Res Ther 5:267, and International Patent Application Publication No. WO2020/245832, the contents of which are fully incorporated herein by reference.

Polymers used in scaffold material compositions may be biocompatible, biodegradable and/or bioerodible and may act as adhesive substrates for cells. In exemplary embodiments, structural scaffold materials are easy to process into complex shapes and have a rigidity and mechanical strength suitable to maintain the desired shape under in vivo conditions.

In certain embodiments, the structural scaffold materials may be non-resorbing or non-biodegradable polymers or materials. Such non-resorbing scaffold materials may be used to fabricate materials which are designed for long term or permanent implantation into a host organism.

The scaffolds may be made by any of a variety of techniques known to those skilled in the art. Salt-leaching, porogens, solid-liquid phase separation (sometimes termed freeze-drying), and phase inversion fabrication may all be used to produce porous scaffolds. Fiber pulling and weaving (see, e.g. Vacanti, et al., (1988) Journal of Pediatric Surgery, 23:3-9) may be used to produce scaffolds having more aligned polymer threads. Those skilled in the art will recognize that standard polymer processing techniques may be exploited to create polymer scaffolds having a variety of porosities and microstructures.

Scaffold materials are readily available to one of ordinary skill in the art, usually in the form of a solution (suppliers are, for example, BDH, United Kingdom, and Pronova Biomedical Technology a.s. Norway). For a general overview of the selection and preparation of scaffolding materials, see the American National Standards Institute publication No. F2064-00 entitled Standard Guide for Characterization and Testing of Alginates as Starting Materials Intended for Use in Biomedical and Tissue Engineering Medical Products Applications”.

The present invention, in some embodiments thereof, also contemplates administration of the pulmonary cells described herein to a subject.

Depending on the application, the method may be effected using pulmonary cells which are syngeneic or non-syngeneic with the subject.

As used herein, the term “syngeneic” cells refer to cells which are essentially genetically identical with the subject or essentially all lymphocytes of the subject. Examples of syngeneic cells include cells derived from the subject (also referred to in the art as an “autologous”), from a clone of the subject, or from an identical twin of the subject.

According to specific embodiments, the pulmonary tissue or cells are non-syngeneic with the subject.

As used herein, the term “non-syngeneic” cells refer to cells which are not essentially genetically identical with the subject or essentially all lymphocytes of the subject, such as allogeneic cells or xenogeneic cells.

As used herein, the term “allogeneic” refers to cells which are derived from a donor who is of the same species as the subject, but which is substantially non-clonal with the subject. Typically, outbred, non-zygotic twin mammals of the same species are allogeneic with each other. It will be appreciated that an allogeneic cell may be HLA identical, partially HLA identical or HLA non-identical (i.e. displaying one or more disparate HLA determinant) with respect to the subject.

As used herein, the term “xenogeneic” refers to a cell which substantially expresses antigens of a different species relative to the species of a substantial proportion of the lymphocytes of the subject. Typically, outbred mammals of different species are xenogeneic with each other.

The present invention envisages that xenogeneic cells are derived from a variety of species. Thus, according to one embodiment, the pulmonary cells are derived from any mammal. Suitable species origins for the pulmonary cells comprise the major domesticated or livestock animals and primates. Such animals include, but are not limited to, porcines (e.g. pig), bovines (e.g., cow), equines (e.g., horse), ovines (e.g., goat, sheep), felines (e.g., Felis domestica), canines (e.g., Canis domestica), rodents (e.g., mouse, rat, rabbit, guinea pig, gerbil, hamster), and primates (e.g., chimpanzee, rhesus monkey, macaque monkey, marmoset).

Pulmonary cells of xenogeneic origin (e.g. porcine origin) are preferably obtained from a source which is known to be free of zoonoses, such as porcine endogenous retroviruses. Similarly, human-derived cells or tissues are preferably obtained from substantially pathogen-free sources.

According to one embodiment, the pulmonary cells are non-syngeneic with the subject.

According to one embodiment, the pulmonary cells are allogeneic with the subject.

According to one embodiment, the pulmonary cells are xenogeneic with the subject.

According to an embodiment of the present invention, the subject is a human being and the pulmonary cells are from a mammalian origin (e.g. allogeneic or xenogeneic).

According to an embodiment of the present invention, the subject is a human being and the pulmonary cells are from a human origin (e.g. syngeneic or non-syngeneic).

According to one embodiment, the subject is a human being and the pulmonary cells are from a xenogeneic origin (e.g. porcine origin).

According to one embodiment, the pulmonary cells may be genetically modified prior to transplantation.

As the pulmonary cells of some embodiments of the present invention comprise progenitor cells having the ability to differentiate into epithelial and endothelial cells, they may be used to treat a pulmonary disorder and/or regenerate a pulmonary tissue in a subject in need thereof.

Thus, according to an aspect of the present invention there is provided a method of regenerating an epithelial and/or endothelial pulmonary tissue in a subject in need thereof, the method comprising administering to the subject a therapeutically effective amount of the isolated population of pulmonary cells disclosed herein, thereby regenerating the epithelial and/or endothelial pulmonary tissue.

According to an additional or an alternative aspect of the present invention, there is provided the isolated population of pulmonary cells disclosed herein for use in regenerating an epithelial and/or endothelial pulmonary tissue in a subject in need thereof.

According to an additional or an alternative aspect of the present invention, there is provided method of treating a pulmonary disorder or injury in a subject in need thereof, the method comprising administering to the subject a therapeutically effective amount of the isolated population of pulmonary cells disclosed herein, thereby treating the pulmonary disorder or injury.

According to an additional or an alternative aspect of the present invention, there is provided the isolated population of pulmonary cells disclosed herein for use in treating a pulmonary disorder or injury in a subject in need thereof.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “subject” or “subject in need thereof” refers to a mammal, preferably a human being, male or female at any age that suffers from or is predisposed to a pulmonary tissue damage or deficiency as a result of a disease, disorder or injury. Typically the subject is in need of pulmonary cell or tissue transplantation (also referred to herein as recipient) due to a disorder or a pathological or undesired condition, state, or syndrome, or a physical, morphological or physiological abnormality which results in loss of organ functionality and is amenable to treatment via pulmonary cell or tissue transplantation.

According to specific embodiments, the subject is a human subject.

As used herein, the phrase “pulmonary disorder or injury” refers to any disease, disorder, condition or to any pathological or undesired condition, state, or syndrome, or to any physical, morphological or physiological abnormality which involves a loss or deficiency of pulmonary cells or tissues or in loss-of-function of pulmonary cells or tissues.

Exemplary pulmonary diseases, include but are not limited to, cystic fibrosis (CF), emphysema, asbestosis, chronic obstructive pulmonary disease (COPD), pulmonary fibrosis, idiopatic pulmonary fibrosis, pulmonary hypertension, lung cancer, sarcoidosis, acute lung injury (adult respiratory distress syndrome), respiratory distress syndrome of prematurity, chronic lung disease of prematurity (bronchopulmonarydysplasia), surfactant protein B deficiency, congenital diaphragmatic hernia, pulmonary alveolar proteinosis, pulmonary hypoplasia, pneumonia (e.g. including that caused by bacteria, viruses, or fungi), asthma, idiopathic pulmonary fibrosis, nonspecific interstitial pneumonitis (e.g. including that present with autoimmune conditions, such as lupus, rheumatoid arthritis or scleroderma), hypersensitivity pneumonitis, cryptogenic organizing pneumonia (COP), acute interstitial pneumonitis, desquamative interstitial pneumonitis, asbestosis, and lung injury (e.g. induced by ischemia/reperfusion pulmonary hypertension or hyperoxic lung injury).

According to one embodiment, the pulmonary disorder or injury comprises chronic inflammation of the lungs (e.g. an inflammation lasting for more than two weeks).

Exemplary chronic inflammation conditions of the lungs include, but are not limited to, chronic airway inflammation, asthma, chronic obstructive pulmonary disease (COPD), lung cancer, cystic fibrosis (CF), granulomatous lung diseases, idiopatic pulmonary fibrosis, chronic lung disease of prematurity, radiation induced pneumonitis, lung diseases associated with systemic diseases such as scleroderma, lupus, dermatomyositis, sarcoidosis, and adult and neonatal respiratory distress syndrome.

According to specific embodiments, the pulmonary disorder or injury is selected from the group consisting of cystic fibrosis, emphysema, asbestosis, chronic obstructive pulmonary disease (COPD), pulmonary fibrosis, idiopatic pulmonary fibrosis, pulmonary hypertension, lung cancer, sarcoidosis, acute lung injury (adult respiratory distress syndrome), respiratory distress syndrome of prematurity, chronic lung disease of prematurity (bronchopulmonarydysplasia), surfactant protein B deficiency, congenital diaphragmatic hernia, pulmonary alveolar proteinosis, pulmonary hypoplasia and asthma.

According to one embodiment, the subject may benefit from transplantation of pulmonary cells or tissues.

According to one embodiment, transplantation of the pulmonary cells results in regenerating of structural/functional pulmonary tissue.

According to one embodiment, transplantation of the pulmonary cells results in generation of a chimeric lung (i.e. a lung comprising cells from genetically distinct origins).

It will be appreciated that the pulmonary cells of some embodiments of the invention are capable of regenerating a structural/functional pulmonary tissue, including generation of a chimeric lung. The chimeric lung comprises alveolar, bronchial and/or bronchiolar structures, and/or vascular structures. Furthermore, the structural/functional pulmonary tissue of some embodiments comprises an ability to synthesize surfactant [e.g. clara cell secretory protein (CCSP), aquqporin-5 (AQP-5) and surfactant protein C (sp-C)], detectable by specific cell staining, and/or an ability to transport ions (e.g. as indicated by staining for CFTR-cystic fibrosis transmembrane regulator). The pulmonary cells of some embodiments of the invention are further capable of regenerating an epithelial, mesenchymal and/or endothelial tissue (e.g. as indicated by the formation of a complete chimeric lung tissue comprising all of these components).

As used herein, the term “regenerating” refers to reconstruction of an epithelial and/or endothelial pulmonary tissue. Thus, in some embodiments of the present invention, regenerating refers to at least about 10%, 20%, 30%, 40%, 50%, 60%, 70%, 80%, 90% or 100% increase in epithelial and/or endothelial tissue. Any method known to one of skill in the art may be used to assess regeneration including for example x-ray, ultrasound, CT, MRI, histological staining of a tissue sample etc.

Following transplantation of the pulmonary cells into the subject according to some embodiments, it is advisable, according to standard medical practice, to monitor the growth functionality and immunocompatability of the transplanted cells according to any one of various standard art techniques. For example, the functionality of a regenerated pulmonary tissue may be monitored following transplantation by standard pulmonary function tests, e.g. by analysis of functional properties of the developing implants, as indicated by the ability to synthesize surfactant, detectable by staining for surfactant protein C (sp-C) and the ability to transport ions, as indicated by staining for CFTR-cystic fibrosis transmembrane regulator.

In order to facilitate engraftment of the pulmonary cells, and in order to reduce, by at least about 30%, 40%, 50%, 60%, 70%, 80%, 90% or 95%, or preferably avoid graft rejection and/or graft versus host disease (GVHD), the method and uses disclosed herein may further advantageously comprise conditioning the subject prior to administration of the pulmonary cells.

As used herein, the term “conditioning” refers to the preparative treatment of a subject prior to transplantation.

According to one embodiment, to increase the rate of a successful transplantation (e.g. the formation of chimerism) the subject is treated by a conditioning capable of vacating cell niches in the pulmonary tissue or organ.

Thus, conditioning the subject is effected by administering to a subject a therapeutically effective amount of an agent capable of inducing damage to the pulmonary tissue wherein the damage results in proliferation of resident stem cells in the pulmonary tissue.

The phrase “damage to the pulmonary tissue” refers to a localized injury to a pulmonary organ/tissue or a part thereof.

The term “proliferation of resident stem cells” refers to the induction of cell division of endogenous stem cells residing within the pulmonary tissue once subjected to the agent.

Various conditioning agents may be used in accordance with the present invention as long as the agent induces damage to at least a part of the pulmonary tissue which results in proliferation of resident stem cells within the pulmonary tissue. Thus, for example, the agent may comprise a chemical, an antibiotic, a therapeutic drug, a toxin or an herb or an extract thereof.

The conditioning protocol may be adjusted taking into consideration the age and condition (e.g. disease, disease stage) of the subject, such a determination is well within the capacity of those of skill in the art, especially in view of the disclosure provided herein.

Without being bound to theory, a therapeutically effective amount of conditioning is an amount of the conditioning agent sufficient for inducing localized pulmonary tissue damage and proliferation of resident stem cells, but not being toxic to other organs of the subject being treated (e.g. liver, kidneys, heart, etc.). Determination of such a therapeutically effective amount is well within the capability of those skilled in the art.

Exemplary agents causing pulmonary cell toxicity, include but are not limited to, chemotherapeutic agents, immunosuppressive agents, amiodarone, beta blockers, ACE inhibitors, nitrofurantoin, procainamide, quinidine, tocainide, minoxidil, amiodarone, methotrexate, taxanes (e.g. paclitaxel and docetaxel), gemcitabine, bleomycin, mitomycin C, busulfan, cyclophosphamide, chlorambucil, nitrosourea (e.g., carmustine) and Sirolimus.

Additional agents causing pulmonary cell toxicity are listed in Table 2, below [incorporated from Collard, www (dot) merckmanuals (dot) com/professional/pulmonary-disorders/interstitial-lung-diseases/drug-induced-pulmonary-disease].

TABLE 2
Substances with toxic pulmonary effects

| Condition | Drug or Agent |
|---|---|
| Asthma | Aspirin, β-blockers (e.g., timolol), cocaine, dipyridamole, IV |
| | hydrocortisone, IL-2, methylphenidate, nitrofurantoin, protamine, |
| | sulfasalazine, vinca alkaloids (with mitomycin-C) |
| Organizing | Amiodarone, bleomycin, cocaine, cyclophosphamide, methotrexate, |
| pneumonia | minocycline, mitomycin-C, penicillamine, sulfasalazine, tetracycline |
| Hypersensitivity | Azathioprine plus 6-mercaptopurine, busulfan, fluoxetine, radiation |
| pneumonitis | |
| Interstitial | Amphotericin B, bleomycin, busulfan, carbamazepine, chlorambucil, |
| pneumonia or | cocaine, cyclophosphamide, diphenylhydantoin, flecainide, heroin, |
| fibrosis | melphalan, methadone, methotrexate, methylphenidate, methysergide, |
| | mineral oil (via chronic microaspiration), nitrofurantoin, nitrosoureas, |
| | procarbazine, silicone (s.c. injection), tocainide, vinca alkaloids (with |
| | mitomycin-C) |
| Noncardiac | β-Adrenergic agonists (e.g., ritodrine, terbutaline), chlordiazepoxide, |
| pulmonary edema | cocaine, cytarabine, ethiodized oil (IV, and via chronic microaspiration), |
| | gemcitabine, heroin, hydrochlorothiazide, methadone, mitomycin-C, |
| | phenothiazines, protamine, sulfasalazine, tocolytic agents, tricyclic |
| | antidepressants, tumor necrosis factor, vinca alkaloids (with mitomycin-C) |
| Parenchymal | Anticoagulants, azathioprine plus 6-mercaptopurine, cocaine, mineral |
| hemorrhage | oil (via chronic microaspiration), nitrofurantoin, radiation |
| Pleural effusion | Amiodarone, anticoagulants, bleomycin, bromocriptine, busulfan, |
| | granulocyte-macrophage colony-stimulating factor, IL-2, methotrexate, |
| | methysergide, mitomycin-C, nitrofurantoin, para-aminosalicylic acid, |
| | procarbazine, radiation, tocolytic agents |
| Pulmonary | Amiodarone, amphotericin B, bleomycin, carbamazepine, |
| infiltrate with | diphenylhydantoin, ethambutol, etoposide, granulocyte-macrophage |
| eosinophilia | colony-stimulating factor, isoniazid, methotrexate, minocycline, |
| | mitomycin-C, nitrofurantoin, para-aminosalicylic acid, procarbazine, |
| | radiation, sulfasalazine, sulfonamides, tetracycline, trazodone |
| Pulmonary | Appetite suppressants (e.g., dexfenfluramine, fenfluramine, |
| vascular disease | phentermine), busulfan, cocaine, heroin, methadone, methylphenidate, |
| | nitrosoureas, radiation |

According to specific embodiments, the agent capable of inducing damage to said pulmonary tissue is selected from the group consisting of a chemotherapeutic agent, an immunosuppressive agent, an amiodarone, a beta blockers, an ACE inhibitor, a nitrofurantoin, a procainamide, a quinidine, a tocainide, and a minoxidil.

According to specific embodiments, the agent capable of inducing damage to the pulmonary tissue comprises naphthalene.

According to one embodiment, naphthalene treatment is administered to the subject 1-10 days (e.g. 7, 6, 5, 4, 3, 2 days, e.g. 3 days) prior to administration of the pulmonary cells.

Assessing pulmonary tissue damage can be carried out using any method known in the art, e.g. by pulmonary function tests, chest X-ray, by chest CT, or by PET scan. Determination of pulmonary damage is well within the capability of those skilled in the art.

As described above, pulmonary tissue damage results in proliferation of resident stem cells within the tissue.

Assessing proliferation of resident stem cells (e.g. endogenous stem cells within a pulmonary tissue) can be carried out using any method know to one of skill in the art, such as for example, by in-vivo imaging of cellular proliferation e.g. using a Positron emission tomography (PET) with a PET tracer e.g. 18F labeled 2-fluoro-2-deoxy-D-glucose (18FDG) or [18F] 3′-deoxy-3-fluorothymidine ((18) FLT) as taught by Francis et al, Gut. (2003) 52 (11): 1602-6 and by Fuchs et al., J Nucl Med. (2013) 54 (1): 151-8.

Thus, according to one embodiment of the invention, following administration of the agent capable of inducing damage to the tissue of interest, the subject is subjected to a second conditioning agent, i.e. an agent which ablates the resident stem cells in the tissue. As will be apparent to those of ordinary skill in the art of cell biology, sensitivity to radiation is achieved only in a proliferative stage.

According to another embodiment, an agent which ablates the resident stem cells in the tissue (as discussed below) can be administered to the subject without prior conditioning with an agent which induces damage to the tissue (e.g. naphthalene).

According to one embodiment, the agent which ablates the resident stem cells comprises a sublethal, lethal or supralethal conditioning protocol.

According to one embodiment, the conditioning protocol comprises reduced intensity conditioning (RIC).

According to an embodiment, the reduced intensity conditioning is effected for up to 2 weeks (e.g. 1-14, 1-10 or 1-7 days) prior to transplantation of the pulmonary cells.

According to one embodiment, the conditioning protocol comprises a total body irradiation (TBI), total lymphoid irradiation (TLI, i.e. exposure of all lymph nodes, the thymus, and spleen), partial body irradiation, T cell debulking (TCD), a chemotherapeutic agent and/or an antibody immunotherapy.

Thus, according to one embodiment, the TBI comprises a single or fractionated irradiation dose within the range of 0.5-1 Gy, 0.5-1.5 Gy, 0.5-2.5 Gy, 0.5-5 Gy, 0.5-7.5 Gy, 0.5-10 Gy, 0.5-15 Gy, 0.5-20 Gy, 1-1.5 Gy, 1-2 Gy, 1-2.5 Gy, 1-3 Gy, 1-3.5 Gy, 1-4 Gy, 1-4.5 Gy, 1-1.5 Gy, 1-7.5 Gy, 1-10, Gy, 1-15, Gy, 1-12 Gy, 2-3 Gy, 2-4 Gy, 2-5 Gy, 2-6 Gy, 2-7 Gy, 2-8 Gy, 2-9 Gy, 2-10 Gy, 2-15 Gy, 2-20 Gy, 3-4 Gy, 3-5 Gy, 3-6 Gy, 3-7 Gy, 3-8 Gy, 3-9 Gy, 3-10 Gy, 3-15 Gy, 3-20 Gy, 4-5 Gy, 4-6 Gy, 4-7 Gy, 4-8 Gy, 4-9 Gy, 4-10 Gy, 4-15 Gy, 4-20 Gy, 5-6 Gy, 5-7 Gy, 5-8 Gy, 5-9 Gy, 5-10 Gy, 5-15 Gy, 5-20 Gy, 6-7 Gy, 6-8 Gy, 6-9 Gy, 6-10 Gy, 6-20 Gy, 7-8 Gy, 7-9 Gy, 7-10 Gy, 7-20 Gy, 8-9, Gy, 8-10 Gy, 10-12 Gy, 10-15 Gy or 10-20 Gy.

According to a specific embodiment, the TBI comprises a single or fractionated irradiation dose within the range of 1-20 Gy.

According to a specific embodiment, the TBI comprises a single or fractionated irradiation dose within the range of 1-10 Gy.

According to an embodiment, TBI treatment is administered to the subject 1-10 days (e.g. 1-3 days) prior to transplantation. According to one embodiment, the subject is conditioned once with TBI 1 or 2 days prior to transplantation.

According to a specific embodiment, the TLI comprises an irradiation dose within the range of 0.5-1 Gy, 0.5-1.5 Gy, 0.5-2.5 Gy, 0.5-5 Gy, 0.5-7.5 Gy, 0.5-10 Gy, 0.5-15 Gy, 0.5-20 Gy, 1-1.5 Gy, 1-2 Gy, 1-2.5 Gy, 1-3 Gy, 1-3.5 Gy, 1-4 Gy, 1-4.5 Gy, 1-1.5 Gy, 1-7.5 Gy, 1-10 Gy, 2-3 Gy, 2-4 Gy, 2-5 Gy, 2-6 Gy, 2-7 Gy, 2-8 Gy, 2-9 Gy, 2-10 Gy, 3-4 Gy, 3-5 Gy, 3-6 Gy, 3-7 Gy, 3-8 Gy, 3-9 Gy, 3-10 Gy, 4-5 Gy, 4-6 Gy, 4-7 Gy, 4-8 Gy, 4-9 Gy, 4-10 Gy, 5-6 Gy, 5-7 Gy, 5-8 Gy, 5-9 Gy, 5-10 Gy, 6-7 Gy, 6-8 Gy, 6-9 Gy, 6-10 Gy, 7-8 Gy, 7-9 Gy, 7-10 Gy, 8-9 Gy, 8-10 Gy, 10-12 Gy, 10-15 Gy, 10-20 Gy, 10-30 Gy, 10-40 Gy, 10-50 Gy, 0.5-20 Gy, 0.5-30 Gy, 0.5-40 Gy or 0.5-50 Gy.

According to a specific embodiment, the TLI comprises a single or fractionated irradiation dose within the range of 1-20 Gy.

According to a specific embodiment, the TLI comprises a single or fractionated irradiation dose within the range of 1-10 Gy.

According to an embodiment, TLI treatment is administered to the subject 1-10 days (e.g. 1-3 days) prior to transplantation. According to one embodiment, the subject is conditioned once with TLI 1 or 2 days prior to transplantation.

According to specific embodiments, the subject may be treated by in-vivo T cell debulking e.g. by anti-CD4 antibody, anti-CD8 antibody, anti-CD3 (OKT3) antibody, anti-CD52 antibody (e.g. CAMPATH) and/or anti-thymocyte globulin (ATG) antibody (e.g. 10, 9, 8, 7, 6 or 5 days prior to transplantation at a therapeutic effective dose of about 100-500 μg, e.g. 300 μg each).

According to one embodiment, the conditioning comprises a chemotherapeutic agent. Exemplary chemotherapeutic agents include, but are not limited to, Busulfan, Myleran, Busulfex, Fludarabine, Melphalan, Dimethyl mileran and Thiotepa and cyclophosphamide. The chemotherapeutic agent/s may be administered to the subject in a single dose or in several doses e.g. 2, 3, 4, 5, 6, 7, 8, 9, 10 or more doses (e.g. daily doses) prior to transplantation. According to one embodiment, the subject is administered a chemotherapeutic agent (e.g. Fludarabine e.g. at a dose of about 30 mg/m²/day) for 3-7 consecutive days, e.g. 5 consecutive days, prior to transplantation (e.g. on days −7 to −3).

According to one embodiment, the conditioning comprises an antibody immunotherapy. Exemplary antibodies include, but are not limited to, an anti-CD52 antibody (e.g. Alemtuzumab sold under the brand names of e.g. Campath, MabCampath, Campath-1H and Lemtrada) and an anti-thymocyte globulin (ATG) agent [e.g. Thymoglobulin (rabbit ATG, rATG, available from Genzyme) and Atgam (equine ATG, cATG, available from Pfizer)]. Additional antibody immunotherapy may comprise anti-CD3 (OKT3), anti-CD4 or anti-CD8 agents. According to one embodiment, the antibody is administered to the subject in a single dose or in several doses e.g. 2, 3, 4, 5, 6, 7, 8, 9, 10 or more doses (e.g. daily doses) prior to transplantation (e.g. 4-8 days, e.g. 6 days, prior to transplantation).

According to one embodiment, the conditioning comprises co-stimulatory blockade. Thus, for example, the conditioning may comprise transiently administering to the subject at least one T-cell co-stimulation inhibitor and at least one CD40 ligand inhibitor, and more preferably may further comprise administering to the subject an inhibitor of T-cell proliferation.

According to one embodiment, the T-cell co-stimulation inhibitor is CTLA4-Ig, the CD40 ligand inhibitor is anti-CD40 ligand antibody, and the inhibitor of T-cell proliferation is rapamycin. Alternately, the T-cell co-stimulation inhibitor may be an anti-CD40 antibody. Alternately, the T-cell co-stimulation inhibitor may be an antibody specific for B7-1, B7-2, CD28, anti-LFA-1 and/or anti-LFA3.

According to a specific embodiment, the conditioning comprises naphthalene treatment (e.g. 10, 9, 8, 7, 6, 5, 4, 3 or 2 days, e.g. 3 days, prior to transplantation) and TBI treatment (e.g. 9, 8, 7, 6, 5, 4, 3, 2 or 1 days, e.g. 1 day, prior to transplantation, at a dose of e.g. 1-20 Gy, e.g. 6 Gy).

According to another specific embodiment, the conditioning comprises T cell debulking treatment (e.g. 10, 9, 8, 7, 6, 5, 4, 3 or 2 days, e.g. 6 days, prior to transplantation, e.g. with anti-CD8 and/or anti-CD4 antibodies), naphthalene treatment (e.g. 10, 9, 8, 7, 6, 5, 4, 3 or 2 days, e.g. 3 days, prior to transplantation) and TBI treatment (e.g. 9, 8, 7, 6, 5, 4, 3, 2 or 1 days, e.g. 1 day, prior to transplantation, at a dose of e.g. 1-20 Gy, e.g. 6 Gy).

According to another specific embodiment, the conditioning comprises only TBI treatment (e.g. 9, 8, 7, 6, 5, 4, 3, 2 or 1 days, e.g. 1 day, prior to transplantation, at a dose of e.g. 1-20 Gy, e.g. 6 Gy).

In order to avoid graft rejection of the pulmonary cells, the subject may be administered with a post-transplant immunosuppressive regimen.

According to one embodiment, the subject is treated with an immunosuppressive regimen for up to two weeks following administration of the pulmonary cells.

According to one embodiment, the subject is treated with an immunosuppressive regimen for up to 5 days, 6 days, 7 days, 8 days, 9 days, 10 days, 11 days, 12 days, 13 days or 14 days following administration of the pulmonary cells.

Examples of suitable types of immunosuppressive regimens include administration of immunosuppressive drugs (also termed immunosuppressive agents) and/or immunosuppressive irradiation.

Ample guidance for selecting and administering suitable immunosuppressive regimens for transplantation is provided in the literature of the art (for example, refer to: Kirkpatrick C H. and Rowlands D T Jr., 1992. JAMA. 268, 2952; Higgins R M. et al., 1996. Lancet 348, 1208; Suthanthiran M. and Strom T B., 1996. New Engl. J. Med. 331, 365; Midthun D E. et al. 1997. Mayo Clin Proc. 72, 175; Morrison V A. et al. 1994. Am J Med. 97, 14; Hanto D W., 1995. Annu Rev Med. 46, 381; Senderowicz A M. et al., 1997. Ann Intern Med. 126, 882; Vincenti F. et al., 1998. New Engl. J. Med. 338, 161; Dantal J. et al. 1998. Lancet 351, 623).

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, tramadol, rapamycin (sirolimus) and rapamycin analogs (such as CCI-779, RAD001, AP23573). These agents may be administered individually or in combination.

According to specific embodiments, the immunosuppressive agent comprises cyclophosphamide, busulfan, fludarabin, tacrolimus, cyclosporine, mycophenolate mofetil, azathioprine, everolimus, sirolimus, glucocorticoids, or combinations thereof.

According to one embodiment, the immunosuppressive agent is cyclophosphamide.

According to one embodiment, the present invention further contemplates administration of cyclophosphamide prior to transplantation (e.g. on days 4, 3 or 2 prior to transplantation, i.e. T-4, -3 or -2) in addition to the administration following transplantation as described herein.

According to specific embodiments, the pulmonary cells administered to the subject comprise an effective amount of hematopoietic precursor cells (HPCs). Alternatively or additionally, according to specific embodiments, the pulmonary cells are administered to a subject in combination with an effective amount of hematopoietic precursor cells (HPCs).

According to specific embodiments, the HPCs may be administered prior to, concomitantly with, or following administration of the pulmonary cells.

As used herein, the term “hematopoietic precursor cells” or “HPCs” refers to a cell preparation comprising immature hematopoietic cells. Such cell preparation includes or is derived from a biological sample, for example, pulmonary tissue (e.g. fetal or adult tissue), bone marrow (e.g. T cell depleted bone marrow), mobilized peripheral blood (e.g. mobilization of CD34+ cells to enhance their concentration), cord blood (e.g. umbilical cord), fetal liver, yolk sac and/or placenta. Additionally or alternatively, purified CD34+ cells or other hematopoietic stem cells, such as CD131+ cells, can be used in accordance with some embodiments of the present teachings, either with or without ex-vivo expansion.

According to specific embodiments, the HPCs comprise pulmonary tissue-derived CD34+ cells.

According to specific embodiments, the HPCs comprise bone marrow or mobilized peripheral blood CD34+ cells.

According to specific embodiments, the HPCs comprise T cell depleted immature hematopoietic cells.

According to specific embodiments, the isolated population of pulmonary cells and the HPCs are obtained from the same donor.

As used herein, the term “an effective amount of HPCs” refers to an amount sufficient to achieve tolerance to the pulmonary cells in the absence of chronic immunosuppressive regimen.

As used herein, the term “tolerance” refers to a condition in which there is a decreased responsiveness of the recipient's cells (e.g. recipient's T cells) when they come in contact with the donor's cells (e.g. donor HPCs) as compared to the responsiveness of the recipient's cells in the absence of such a treatment method.

Tolerance induction enables transplantation of a cell or tissue graft (e.g. pulmonary cells) with reduced risk of graft rejection or graft versus host disease (GVHD).

An effective amount of HPCs typically comprise about 1×10⁵-10×10⁷ cells per Kg body weight of the subject.

The cells of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the pulmonary cells accountable for the biological effect.

Hence, according to an aspect of the present invention, there is provided a pharmaceutical composition comprising as an active ingredient the isolated population of pulmonary cells and a pharmaceutical acceptable carrier.

According to specific embodiments, the pharmaceutical composition further comprises hematopoietic precursor cells (HPCs) as an active ingredient.

According to specific embodiments, the isolated population of pulmonary cells and the hematopoietic precursor cells (HPCs) are in separate formulations.

According to other specific embodiments, the isolated population of pulmonary cells and the HPCs are in the same formulation.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac e.g., into the right or left ventricular cavity, into the common coronary artery, intravenous, intratracheal, intrabronchial, intralveolar, intraperitoneal, intranasal, or intraocular injections.

According to one embodiment, administering is effected by an intravenous route.

Thus, according to specific embodiments, the cells are formulated for intravenous administration.

According to one embodiment, administering is effected by an intratracheal route.

Thus, according to specific embodiments, the cells are formulated for intratracheal administration.

Alternatively, administration of the pulmonary cells to the subject may be effected by administration thereof into various suitable anatomical locations so as to be of therapeutic effect. Thus, depending on the application and purpose, the pulmonary cells may be administered into a homotopic anatomical location (a normal anatomical location for the organ or tissue type of the cells), or into an ectopic anatomical location (an abnormal anatomical location for the organ or tissue type of the cells).

Accordingly, depending on the application and purpose, the pulmonary cells may be advantageously implanted (e.g. transplanted) under the renal capsule, or into the kidney, the testicular fat, the sub cutis, the omentum, the portal vein, the liver, the spleen, the heart cavity, the heart, the chest cavity, the lung, the pancreas, the skin and/or the intra-abdominal space.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient (e.g. pulmonary tissue).

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. pulmonary cells) effective to prevent, alleviate or ameliorate symptoms of a disorder (e.g., pulmonary disease or condition) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

An exemplary animal model which may be used to evaluate the therapeutically effective amount of pulmonary cells comprises the murine animal model (e.g. mice), in which lung injury is induced by e.g. intraperitoneal injection of naphthalene (e.g. more than 99% pure) with or without further irradiation (e.g. 40-48 hours after naphthalene administration), as described in detail in the Examples section which follows. For Example, an immune deficient mouse such as NOD-SCID mouse may be treated with naphthalene followed by irradiation (e.g. low does TBI e.g. 1-3 Gy) followed by administration of the pulmonary cells.

According to specific embodiments, the pulmonary cells are administered to the subject at a dose range of about 1-10×10⁶, 5-10×10⁶, 1-50×10⁶, 10-50×10⁶, 10-60×10⁶, 10-70×10⁶, 10-80×10⁶, 10-90×10⁶, 1-100×10⁶, 5-100×10⁶, 10-100×10⁶, 40-100×10⁶, 50-100×10⁶, 1-200×10⁶, 5-200×10⁶, 10-200×10⁶, 50-200×10⁶, 100-200×10⁶, 1-500×10⁶, 5-500×10⁶, 10-500×10⁶, 100-500×10⁶, 1-1000×10⁶, 5-1000×10⁶, 10-1000×10⁶, 40-1000×10⁶, 50-1000×10⁶, 100-1000×10⁶, 500-1000×10⁶, 1-2000×10⁶, 5-2000×10⁶, 10-2000×10⁶, 20-2000×10⁶, 30-2000×10⁶, 40-2000×10⁶, 50-2000×10⁶, 60-2000×10⁶, 70-2000×10⁶, 80-2000×10⁶, 90-2000×10⁶, 100-2000×10⁶, 200-2000×10⁶, 300-2000×10⁶, 400-2000×10⁶, 500-2000×10⁶, 600-2000×10⁶, 700-2000×10⁶, 800-2000×10⁶, 900-2000×10⁶, 1000-2000×10⁶, 1500-2000×10⁶, 100-3000×10⁶, 200-3000×10⁶, 300-3000×10⁶, 400-3000×10⁶, 500-3000×10⁶, 600-3000×10⁶, 700-3000×10⁶, 800-3000×10⁶, 900-3000×10⁶, 1000-2000×10⁶, 1000-3000×10⁶, 2000-3000×10⁶, 500-4000×10⁶, 1000-4000×10⁶, 2000-4000×10⁶, 3000-4000×10⁶ cells per Kg body weight of the subject.

According to a specific embodiment, CD45 depleted lung cells expanded in culture and comprising about 1-10% cells being double positive for expression of epithelial and endothelial cell markers are administered to the subject at a dose range of about 1-10×10⁶, 5-10×10⁶, 1-50×10⁶, 10-50×10⁶, 10-60×10⁶, 10-70×10⁶, 10-80×10⁶, 10-90×10⁶, 1-100×10⁶, 5-100×10⁶, 10-100×10⁶, 40-100×10⁶, 50-100×10⁶, 1-200×10⁶, 5-200×10⁶, 10-200×10⁶, 50-200×10⁶, 100-200×10⁶, 1-500×10⁶, 5-500×10⁶, 10-500×10⁶, 100-500×10⁶, 1-1000×10⁶, 5-1000×10⁶, 10-1000×10⁶, 40-1000×10⁶, 50-1000×10⁶, 100-1000×10⁶, 500-1000×10⁶, 1-2000×10⁶, 5-2000×10⁶, 10-2000×10⁶, 20-2000×10⁶, 30-2000×10⁶, 40-2000×10⁶, 50-2000×10⁶, 60-2000×10⁶, 70-2000×10⁶, 80-2000×10⁶, 90-2000×10⁶, 100-2000×10⁶, 200-2000×10⁶, 300-2000×10⁶, 400-2000×10⁶, 500-2000×10⁶, 600-2000×10⁶, 700-2000×10⁶, 800-2000×10⁶, 900-2000×10⁶, 1000-2000×10⁶, 1500-2000×10⁶, 100-3000×10⁶, 200-3000×10⁶, 300-3000×10⁶, 400-3000×10⁶, 500-3000×10⁶, 600-3000×10⁶, 700-3000×10⁶, 800-3000×10⁶, 900-3000×10⁶, 1000-2000×10⁶, 1000-3000×10⁶, 2000-3000×10⁶, 500-4000×10⁶, 1000-4000×10⁶, 2000-4000×10⁶, 3000-4000×10⁶ cells per Kg body weight of the subject.

According to an embodiment of the present invention, the pulmonary cells are administered to the subject at a dose of at least about 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 10×10⁶, 12.5×10⁶, 15×10⁶, 20×10⁶, 25×10⁶, 30×10⁶, 35×10⁶, 40×10⁶, 45×10⁶, 50×10⁶, 60×10⁶, 70×10⁶, 80×10⁶, 90×10⁶, 100×10⁶, 110×10⁶, 120×10⁶, 130×10⁶, 140×10⁶, 150×10⁶, 160×10⁶, 170×10⁶, 180×10⁶, 190×10⁶, 200×10⁶, 250×10⁶, 300×10⁶, 320×10⁶, 350×10⁶, 400×10⁶, 450×10⁶, 500×10⁶, 600×10⁶, 700×10⁶, 800×10⁶, 900×10⁶, 1000×10⁶, 1100×10⁶, 1200×10⁶, 1300×10⁶, 1400×10⁶, 1500×10⁶ or 2000×10⁶ cells per kilogram body weight of the subject.

According to a specific embodiment, lung cells expanded in culture and comprising cells selected or enriched (e.g. at least 20%) for being double positive for expression of epithelial and endothelial cell markers (prior to or following the culture) are administered to the subject at a dose range of about 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 10×10⁶, 12.5×10⁶, 15×10⁶, 20×10⁶, 25×10⁶, 30×10⁶, 35×10⁶, 40×10⁶, 45×10⁶, 50×10⁶, 60×10⁶, 70×10⁶, 80×10⁶, 90×10⁶, 100×10⁶, 110×10⁶, 120×10⁶, 130×10⁶, 140×10⁶, 150×10⁶, 160×10⁶, 170×10⁶, 180×10⁶, 190×10⁶, 200×10⁶, 250×10⁶, 300×10⁶, 320×10⁶, 350×10⁶, 400×10⁶, 450×10⁶, 500×10⁶, 600×10⁶, 700×10⁶, 800×10⁶, 900×10⁶, 1000×10⁶, 1100×10⁶, 1200×10⁶, 1300×10⁶, 1400×10⁶, 1500×10⁶ or 2000×10⁶ cells per kilogram body weight of the subject.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide ample levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

To further avoid residual immune reaction which may still be present when administering pulmonary cells, several approaches have been developed to reduce the likelihood of rejection.

These include encapsulating the non-syngeneic cells in immunoisolating, semipermeable membranes before transplantation. Alternatively, cells may be uses which do not express xenogenic surface antigens, such as those developed in transgenic animals (e.g. pigs).

Encapsulation techniques are generally classified as microencapsulation, involving small spherical vehicles, and macroencapsulation, involving larger flat-sheet and hollow-fiber membranes (Uludag, H. et al. (2000). Technology of mammalian cell encapsulation. Adv Drug Deliv Rev 42, 29-64).

Methods of preparing microcapsules are known in the art and include for example those disclosed in: Lu, M. Z. et al. (2000). Cell encapsulation with alginate and alpha-phenoxycinnamylidene-acetylated poly(allylamine). Biotechnol Bioeng 70, 479-483; Chang, T. M. and Prakash, S. (2001) Procedures for microencapsulation of enzymes, cells and genetically engineered microorganisms. Mol Biotechnol 17, 249-260; and Lu, M. Z., et al. (2000). A novel cell encapsulation method using photosensitive poly(allylamine alpha-cyanocinnamylideneacetate). J Microencapsul 17, 245-521.

For example, microcapsules are prepared using modified collagen in a complex with a ter-polymer shell of 2-hydroxyethyl methylacrylate (HEMA), methacrylic acid (MAA), and methyl methacrylate (MMA), resulting in a capsule thickness of 2-5 μm. Such microcapsules can be further encapsulated with an additional 2-5 μm of ter-polymer shells in order to impart a negatively charged smooth surface and to minimize plasma protein absorption (Chia, S. M. et al. (2002). Multi-layered microcapsules for cell encapsulation. Biomaterials 23, 849-856).

Other microcapsules are based on alginate, a marine polysaccharide (Sambanis, A. (2003). Encapsulated islets in diabetes treatment. Diabetes Thechnol Ther 5, 665-668), or its derivatives. For example, microcapsules can be prepared by the polyelectrolyte complexation between the polyanions sodium alginate and sodium cellulose sulphate and the polycation poly (methylene-co-guanidine) hydrochloride in the presence of calcium chloride.

It will be appreciated that cell encapsulation is improved when smaller capsules are used. Thus, for instance, the quality control, mechanical stability, diffusion properties, and in vitro activities of encapsulated cells improved when the capsule size was reduced from 1 mm to 400 μm (Canaple, L. et al. (2002). Improving cell encapsulation through size control. J Biomater Sci Polym Ed 13, 783-96). Moreover, nanoporous biocapsules with well-controlled pore size as small as 7 nm, tailored surface chemistries, and precise microarchitectures were found to successfully immunoisolate microenvironments for cells (See: Williams, D. (1999). Small is beautiful: microparticle and nanoparticle technology in medical devices. Med Device Technol 10, 6-9; and Desai, T. A. (2002). Microfabrication technology for pancreatic cell encapsulation. Expert Opin Biol Ther 2, 633-646).

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., cd. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Mice—Animals were maintained under conditions approved by the Institutional Animal Care and Use Committee at the Weizmann Institute and MD Anderson Cancer Center. All of the procedures were monitored by the Veterinary Resources Unit of the Weizmann Institute and MDA, and approved by the Institutional Animal Care and Use Committee (IACUC). Mice strains used included: Rag1^{-/-} (on C57BL/BJ background), C57BL/6J (CD45.2) and C57BL/6-Tg (CAG-EGFP) 1Osb/J, (Weizmann Institute Animal Breeding Center, Rchovot, Israel), Gt(ROSA)26Sortm4 (ACTB-tdTomato,-EGFP) Luo/J (39), B6.129P2-Gt (ROSA) 26Sortml (CAG-Brainbow2.1) Clc/J (9), B6.Cg-Shh^{tm1(EGFP/cre) Cjt}/J (40), B6.129-Tg (Cdh5-cre) 1Spe/J (21), B6.Cg-Ager^{tm2.1(cre/ERT2)Blh}/J (23), Hopx^{tm2.1(cre/ERT2)Joe}/J (35), B6N. 129S6-Gt(ROSA)26Sor^{tm1(CAG-tdTomato*,-EGFP*)Ees}/J (41), Nkx2-1tm1.1 (cre/ERT2) Zjh/J (22), (Jackson labs, Bar Harbor, USA). All mice were used at 6-12 weeks of age. Mice were kept in small cages (up to five animals in each cage) and fed sterile food and acid water. Randomization: Animals of the same age, sex and genetic background were randomly assigned to treatment groups. Pre-established exclusion criteria were based on IACUC guidelines, and included: systemic disease, toxicity, respiratory distress, interference with eating and drinking, substantial (above 15%) weight loss. During the entire study period, most of the animals appeared to be in good health, and were included in the appropriate analysis. In all experiments the animals were randomly assigned to the treatment groups.

Naphthalene lung injury—Naphthalene (>99% pure; Sigma-Aldrich) was dissolved in corn oil, and administered at a dose of 200 mg per kg body weight, by intra-peritoneal injection, 40-48 hours before exposure to TBI, as previously described (6-8). For “double” lung injury, naphthalene treated animals were further irradiated in an X-Ray Irradiator (40-48 hrs after naphthalene administration). C57BL/6, and Rag1/mice were irradiated with 6Gy TBI.

Lung cells suspensions—Fetal or adult lung cell suspensions were obtained by enzymatic digestions, as previously described (6-8), with some modifications. Briefly, lung digestion was performed by either finely mincing tissue with a razor blade in the presence of 1% collagenase, 2.4 U/ml Dispase II (Roche Diagnostics, Indianapolis, IN) 1 mg/ml DNAse-I (Roche Diagnostics, Indianapolis, IN) in PBS Ca⁺Mg⁺, or by enzymatic digestion of the lung tissues in the presence of 1 mg/ml collagenase, 2.4 U/ml Dispase II and 1 mg/ml DNAse-I (Roche Diagnostics) in Ca⁺Mg⁺ phosphate buffered saline in GentleMACS™ Octo Dissociator with Heaters (Miltenyi Biotec), using mouse lung dissociation protocol provided by the vendor. Removal of nonspecific debris was accomplished by sequential filtration through 70- and 40-μm filters or 100-, 70- and 40 μm filters. The cells were then washed with PBS including 2% FCS, 2 mM EDTA, and antibiotics.

Liver cells suspensions—Fetal or adult livers were placed in 40 μM cell strainers, mashed using the flat end of a plunger from a 5 ml syringe in the 6-well plates and dispensed in 3-4 mL of ice-cold DPBS. Following, the cells were rinsed with ice-cold DPBS to collect the filtered cell suspensions into 15 μL conical tubes. The cells were collected by centrifugation in a chilled centrifuge (1,300 rpm, for 10 minutes) and resuspended in 3-5 mL DPBS prior to injection. The cells were counted, using trypan blue and resuspended to make a stock solution of 10⁷ cells/mL.

Bone marrow cells suspensions—Bone marrow cells were prepared for transplantation by crushing the long bones of donor mice, using a mixer homogenizer (OMNI). The cells were depleted of CD4 and CD8 cells, using a Miltenyi magnetic microbead separation protocol, according to the manufacturer's instructions.

Fetal and adult lung cells transplantation—C57BL/6 mice or Rag1^{-/-} recipient mice were conditioned with naphthalene, and 48 hours later exposed to 6Gy TBI. The mice were transplanted with suspended 1×10⁶ E15-E16 embryonic or 3-16×10⁶ adult mouse lung cells by injection into the tail vein 4-8 hours following irradiation.

TMX administration—TMX (Sigma-Aldrich) was prepared in corn oil as a 20 mg/ml stock solution. For induction of Cre recombination in R26R-Confetti adult mice, the mice were injected intraperitoneally (IP) with two doses of TMX-5 mg on days 5 and 4 prior to harvest of bone marrow or lungs. Lungs or BM were harvested 5 days after TMX administration. For induction of CRE recombination in Confetti embryos, female mice were treated at E12 with a single IP dose of 5 mg TMX. The embryos were harvested at E16, and lung and liver cells of the Confetti-positive embryos were used for transplantation experiments.

For induction of Cre recombination in Nkx2-1tm1.1 (cre/ERT2) Zjh/J mTmG, B6.Cg-Ager^{tm2.1(cre/ERT2)Blh}/mTmG, and Hopx^{tm2.1(cre/ERT2)Joe}/J mice mTmG, 4 mg of TMX was injected 6 days prior to harvesting the lungs for FACS analysis.

Spleen colony assay—Bone marrow was harvested by flushing long bones of R26R-Confetti 8-week-old mice with ice cold PBS, 5 days after TMX administration, and BM cells were transplanted into lethally irradiated (10GyTBI) mice by tail vein injection. Nine days after transplantation, the mice were sacrificed and the spleens were evaluated for the presence of spleen colonies by binocular and fluorescent microscopy.

Flow cytometry analysis of R26R-Confetti donor cells and transgenic mouse lungs—FACS analysis was performed on LSRII (BD Biosciences) or Fortessa analyzers equipped with 5 lasers. E16 fetal Confetti lung and liver cell and adult Confetti lungs and bone marrow cells were analyzed. E16 fetal and adult lung cells were analyzed for YFP/GFP, RFP and CFP labeled cells as well as for epithelial (CD326), endothelial (CD31), and hemopoietic (CD45) lineage markers, to quantify the different cell populations within the cells marked with different fluorescent tags after Cre recombination, induced by TMX administration.

Samples were stained with the conjugated antibodies or matching isotype controls according to the manufacturer's instructions: the full list of anti-CD326, anti-CD31 and anti-CD45 antibodies is provided in Table 3 hereinbelow. E16 fetal liver and adult bone marrow cells were analyzed for the Lin⁻ Sca-1⁺c-kit⁺ cell (LSK) population. The cells were stained with the following antibodies or matching isotype controls: Lineage panel-streptavidin, followed by staining with biotin-APC or biotin APC-CY7, Sca-1-Brilliant Violet 711, C-kit PE-CY7. Full list of antibodies used is provided in Table 3 hereinbelow. Antibodies were purchased from e-Bioscience, BD, and Biolegend.

Data were analyzed using FlowJo software (version vX.0.7 Tree Star Inc).

Image acquisition by TPLSM—A Zeiss LSM 880 upright microscope fitted with Coherent Chameleon Vision laser was used for explant lung tissue imaging experiments. Images were acquired with a femtosecond-pulsed two-photon laser tuned to 940 nm. The microscope was fitted with a filter cube containing 565 LPXR to split the emission to a PMT detector (with a 579-631 nm filter for tdTomato fluorescence) and to an additional 505 LPXR mirror to further split the emission to 2 GaAsp detectors (with a 500-550 nm filter for GFP fluorescence). Images were acquired as 100-150 μm Z-stacks with 1-5 μm steps between each Z-plane. The zoom was set to 0.7, and pictures were acquired at 512×512 x-y resolution.

Tissue clearing—Mice were perfused with monomer solution containing 4% PFA, 4% acrylamide, 0.0125% bis-acrylamide, and 0.1% azo-initiator, VA-044. The lungs were immediately immersed in the above solution for additional 24-48 hours, at 40° C. with constant shaking, to avoid premature polymerization. After degassing, the lungs were left to polymerize for 3 hours at 37° C. and washed with 20 mM sodium borate buffer containing 200 mM SDS at pH=9. The lungs were then cleared for 4 days using a rotational electrophoresis clearing system (the SmartClear II Pro, Life Canvas Technologies, Seoul, South Korea). Once the lungs reached a sufficient clearing state, they were washed in 20 mM sodium borate buffer for 24 hours and immersed in index matching solution until imaging (EasyIndex; RI=1.46, Lifecanvas technologies).

Light-Shect microscopy—For imaging of large lung volume, three-dimensional images of cleared lungs were acquired using an ultramicroscope II (LaVision BioTec GmbH, Astastraße 14, 33617 Bielefeld/Germany) operated by the InspectorPro software (LaVision BioTec). The light sheet was generated by a Superk Super-continuum white light laser with an emission range of 460-800 nm, 1 mW/nm (NKT photonics, Blokken 84, DK-3460 Birkerød). Excitation filters used to detect red and green patches were 545/25 and 470/40, respectively. The corresponding emission filters were 595/40 and 525/50. The microscope was equipped with a single lens configuration, 4× objective (LVMI-Fluor 4×/0.3; NA: 0.3; WD: 5.6-6.0 mm; RI range: 1.33-1.57). Samples were glued to the sample holder and placed in an imaging chamber made of 100% quartz (LaVision BioTec) filled with EasyIndex solution (RI=1.46; lifecanvas technologies) and illuminated from the side. Images were acquired by an Andor Neo sCMOS camera (16 bit, 2150×2560, pixel size 1.626×1.626 μm, Andor 277.3 mi. Belfast, UK). Z stacks were acquired with 5 μm steps, and two fields of view, 3510×4160 μm each, were acquired with 20% overlap, and stitched using Imaris stitcher (BITPLANE by Oxford Instrument, www(dot)bitplanc(dot)com).

To increase the imaging resolution of the red and green engrafted lung patches in 3D, samples were also imaged using a LightSheet Z.1 microscope equipped with 2 sCMOS cameras PCO.Edge, 1920×1920 (Zeiss Ltd, Tegeluddsvägen 76 115 28 Stockholm, Sweden). The red and green patches were illuminated using Zeiss illumination optics lightsheet 10×/0.2 and their emission detected using Clr Plan-Neofluar 20×/1.0 Corr nd=1.45 (Zeiss Ltd.). Samples were glued at their edge to a designated holder, and immersed into the imaging chamber, filled with EasyIndex solution (Lifecanvas Technologies).

Imaging was performed using single side illumination, at two fields of view: 1093.91×1093.91×296.22 μm, and 437.56×437.56×315.33 μm. The excitation lines for the red and green patches were 561 nm at 1% and 488 nm at 2%, with collected emission at 575-615 nm and 505-545 nm, respectively.

Assessment of R26R-Confetti⁺ foci in chimeric lungs by immunohistology—Lungs were fixed with a 4% PFA solution introduced through the trachea under a constant pressure of 20 cm H₂O. Then, the lungs were immersed in fixative overnight at 4° C. Lungs were processed after PFA treatment and fixed in 30% sucrose and frozen in Optimal Cutting Temperature (OCT) compound (Sakura Finetek USA, Inc.Tissue-Tek). Serial step sections, 12 μm in thickness, were taken along the longitudinal axis of the lobe. The fixed distance between the sections was calculated so as to allow systematic sampling of at least 20 sections across the whole lung. Lung slices were analyzed by fluorescence microscopy or confocal microscopy. The actual number of “Confetti” patches (a group of more than 5 adjacent cells labelled with the same fluorescent tag: cytoplasmatic RFP and YFP, nuclear GFP and membrane CFP, was defined as a single patch) was counted per slice.

Confocal Microscopy-Engrafted lung thin 12 μm sections were imaged using an upright laser scanning Leica TCS SP8 microscope, equipped with acousto optical beam splitter and acousto optical tunable filter (Leica microsystems CMS GmbH, Germany) for wavelength separation and 2 internal HyD detectors equipped with spectral separation. The confocal pinhole was open to 1AU (58.6 μm for 580 nm). Images were acquired using the 8k Hz resonant scanner in a 1024*1024, 8 bit format at two magnifications. Use of 20× air objective (HC PL 20×/0.75 W, Leica microsystems), provided images with a field of view of 443.29 μm; pixel size=0.43 μm. For higher resolution images, a 60× oil objective was used (HC PL APO 63×/10⁴ oil CS2, Leica microsystems) providing image dimensions of FOV=140.73 μm; pixel size=0.137 μm. For well distinguished excitation and signal collection from the 5 different markers (see the list of antibodies and staining details) sequential imaging steps were applied, with sequence shift following each Z stack acquisition: the 1^{st} sequence applied excitation with an Ar laser at 2% (of 30% laser) at 488 nm, and HeNe633 laser at 1%, with emission collected at 505-566 nm and 651-708 nm, respectively. The 2^{nd} sequence applied excitation using DPSS561 laser at 4% and collection at 574-638 nm. The 3^{rd} sequence applied excitation with Diode405 laser at 8% and collected two emissions at 413-446 nm and 493-521 nm.

Wide-Field Microscopy—To image the entire area of a lung tissue section, a Leica DMi8 inverted microscope was used equipped with a motorized stage for fast imaging. Imaging was done with a 10× air objective (HC PL FLUOTAR 10×/0.3 DRY, Leica microsystems) and recorded with a CCD camera (1392×1040, 8 bit, Leica DFC7000 GT monochromatic, Leica microsystems).

Transplantation of sorted CD326+CD31+, CD326+, CD31+ cell populations into NA+6Gy TBI preconditioned mice—Experiments were performed, involving transplantation of FACS-sorted cell populations from adult mouse lungs. Labeled donor mice used included GFP (C57BL/6-Tg (CAG-EGFP)1Osb/J), mTmG (Gt (ROSA)26Sor^{tm4(ACTB-tdTomato,-EGFP)Luo}/J) and nTnG (Gt(ROSA)26Sor^{tm1(CAG-tdTomato*,-EGFP*)Ees}/J) (mTmG mice express membrane td-tomato, and nTnG mice express nuclear td-tomato). Altogether, a total of 16 independent transplantation experiments, testing the patch-forming activity of sorted cells were performed. Live, single CD45-lung cells were FACS sorted into four subpopulations including CD326+CD31-, CD326+CD31+, CD326−CD31+ and CD326−CD31− cells. The 0.3-0.5×10⁶ sorted cells from either mTmG or nTnG donors were transplanted with or without 0.5×10⁶ unseparated lung cells from GFP+ mouse donors into naphthalene treated and irradiated C57BL mice. The lungs of transplanted mice were harvested and evaluated for the presence of donor-derived cells 6 to 24 weeks post-transplantation.

Single-molecule fluorescent in situ hybridization (smFISH)-SmFISH was performed as previously described (42). For smFISH, lung tissues were harvested inflated and fixed in 4% paraformaldehyde for 3 hours; incubated overnight with 30% sucrose in 4% paraformaldehyde, and then embedded in OCT and frozen. 6 μm cryosections were used for hybridization. smFish probes were coupled to Cy5. The probes were purchased from Stellaris® Bioscarch™ Technologies.

Single-cell RNAseq analysis of the chimeric lungs-Chimeric lungs were harvested from C57B1 mice transplanted with TdTom single cell suspension, 6 months post-transplant. The lungs from three chimeric mice were enzymatically treated, dissociated into single cells, pooled, and FACS sorted for CD45-td-tomato positive and CD45-td-tomato negative cells. The single cell RNA transcriptome analysis was performed at the MD Anderson core lab, using 10× Genomics platform. The Cell Ranger Single Cell Software Suite v3.0.1 (www(dot)support(dot)10xgenomics(dot)com/single-cell-gene-expression/software/overview/welcome) was used for sample demultiplexing, alignment to the mm10 mouse reference genome and transcriptome, and generating filtered unique molecular identifier (UMI) count matrices, which were used for downstream analyses. The R package Seurat (43-45) v3.1.1 was used to perform data QC, normalization, and integration of the three datasets. Cells with dataset-specific outliers of high mitochondria percentage, extremely high or low number of genes, or high RNA content were filtered out as potential dead cells or doublets. LogNormalize was used for normalization. Integration was based on 2000 anchor genes as a default. The R package monocle3 (46-48) v0.2.0 was used for dimension reduction of the integrated dataset using the uniform manifold approximation and projection (UMAP) method (49) and unsupervised clustering of cells was performed using Louvain/Leiden community detection (50). Marker genes of each cluster were identified using Seurat by comparing the gene expression profile of each cluster to all others using Wilcoxon rank sum test. A single-sample gene set variation analysis was performed to calculate cluster-wise gene set enrichment scores using R package GSVA (51) v1.32.0 on MSigDB (52) hallmark gene sets (v7.0) and curated lung gene sets (www(dot)research(dot)cchmc(dot)org/pbge/lunggens/mainportal(dot)html). The resulting clusters were manually curated and/or merged based on marker genes and gene set analysis results.

Epithelial cell colony forming assay-lung organoids were grown by culturing FACS sorted cells from VEcad mTmG adult mouse lungs in 50% growth factor-reduced (GFR) Matrigel (BD Biosciences), on IBIDI u-slides with glass bottom (Cat.No: 81507). For cell sorting experiments lung single cell suspensions were prepare by enzymatic digestion of the lung tissues in the presence of 1 mg/ml collagenase, 2.4 U/ml dispase and 1 mg/ml DNAse-I (Roche Diagnostics) in Ca⁺Mg⁺ phosphate buffered saline in GentleMACS™ Octo Dissociator with Heaters (Miltenyi Biotec), using mouse lung dissociation protocol provided by the vendor. Sigle cells were stained with the conjugated antibodies for CD45, CD326 and CD31. CD326+ mG− and CD326+mG+ cells are purified by FACS, mixed with Matrigel and 10 μl of cell/Matrigel mixture containing 4-5×10⁵ cells is cultured per well. After few hours of solidification of the gel, 50 μl of epithelial growth medium was added on top of the cultured cells. The medium was replaced every 48-72 hours. The absolute number of epithelial clones was determined after 7-10 days in culture; in some experiments colony-forming efficiency was calculated as the number of growing clones per seeded cell number ×100, as a percentage (number of seeded cells that gave rise to growing clones divided by the total number of cells seeded in the well). All cell cultures were carried out at 37° C. in a 5% CO2 humidified incubator. After 7-10 days in culture the whole mount epithelial colonies are stained and analyzed by confocal microscopy (Olympus FV3000).

Statistical analysis and reproducibility-Sample size calculations were not performed. Both female and male mice were used as donors and recipients. Fetal lungs used for transplantation experiments were harvested at embryonic day E16. Adult lung donors used for transplantation of either unseparated or FACS purified populations were 6-12 weeks old. All the experiments were conducted in least in 3 biological replicates. All data are presented as the mean±SEM or SD. Statistical analyses were performed using Prizm software. Comparisons were tested using Student's t test, X2 distribution test or one-way analysis of variance (ANOVA). P value of <0.05 was considered to be significant. All graphs were generated using Excel, Prizm and Adobe Illustrator CC 2018 software.

Lung cells culture-conditioned medium (CM) from irradiated mouse embryonic fibroblasts (iMEF) was collected every 24-48 hours. Lungs were freshly harvested from 6-12 weeks old C57BL/6 and enzymatically digested into single cell suspension as described hereinabove. Following, CD45+ cells were depleted by magnetic beads (CD45 microbeads, Miltenyi Biotec, #130-052-301), according to the manufacturer's instruction. 3×10⁶ cells per 10 ml CM or 3×10⁵ cells per 1/6 well tissue culture plate were then resuspended in CM supplemented with epidermal growth factor (hEGF, Stemcell, #78006 (20 μg/ml) and ROCK-inhibitor (Y-27632, Tocris, #1254, (5 μm) or with epidermal growth factor (EGF) (20 μg/ml), ROCK-inhibitor (RI) (20 μm) and vascular endothelial growth factor (hVEGF, Stemcell, #78073), and cultured in a 6 well plate (3×10⁵ cells per well) at 37° C. in a 5% CO₂ humidified incubator. Medium was replaced every 48-72 hours. Cells were passaged, counted, and stained with anti-CD31 and anti-CD326 antibodies (see Table 3 here in below) for FACS analysis every 72-96 hours to track cells growth.

TABLE 3
list of antibodies

| | Application | Catalog No. | Dilution | |
|---|---|---|---|---|
| Primary antibodies | | | | |
| Rabbit anti- ERG (Abcam) | IHC | AB-92513 | 1:100 | |
| Rabbit anti- Nkx2.1 (Abcam) | IHC | AB-76013 | 1:100 | |
| Rabbit anti- wide spectrum cytokeratin | IHC | Z0622 | 1:100 | |
| (Dako) | | | | |
| Rabbit anti- surfactant protein C (Santa- | IHC | Sc-13979 | 1:100 | |
| Cruz) | | | | |
| Rabbit anti- Aquaporin5 (Millipore) | IHC | CALBIOCHE | 1:150 | |
| | | M 178615 | | |
| Rat anti-mouse E-cadherin ECCD-2 | IHC | 13-1900 | 1:100 | |
| (Invitrogen) | | | | |
| Goat anti-mouse CD31 (RD SYSTEMS) | IHC | AF 3628 | 1:100 | |
| Rabbit anti SOX17 (Abcam) | IHC | Ab 224637 | 1:100 | |
| Guinea pig anti-mouse LAMP3 SYSY | IHC | 391005 | 1:1000 | |
| (Synaptic Systems) | | | | |
| Rat anti- mouse CD31(Dianova) | IHC | DIA-310-M | 1:50-100 | |
| | | Clone SZ31 | | |
| Rabbit anti-HOPX | IHC | HPA030180 | 1:100 | |
| Anti- mouse Sca APC-Cy7 (Biolegend) | FACS | 108126 | 1 | ul/10⁶ cells |
| Anti- mouse Sca Pacific Blue (Biolegend) | FACS | 108120 | 1 | ul/10⁶ cells |
| Anti- mouse Sca APC (Biolegend) | FACS | 108112 | 1 | ul/10⁶ cells |
| Anti- mouse Sca Brilliant Violet 711 | FACS | 108131 | 1 | ul/10⁶ cells |
| (Biolegend) | | | | |
| Anti- mouse CD45 APC-Cy7 (Biolegend) | FACS | 103116 | 1 | ul/10⁶ cells |
| Anti- mouse CD45 PE (Biolegend) | FACS | 103106 | 1 | ul/10⁶ cells |
| Anti- mouse CD31 APC (Biolegend) | FACS | 102510 | 1 | ul/10⁶ cells |
| Anti- mouse CD31 PE-Cy7 (Biolegend) | FACS | 102418 | 1 | ul/10⁶ cells |
| Anti- mouse CD31 PE (Biolegend) | FACS | 102408 | 1 | ul/10⁶ cells |
| Anti- mouse CD31 FITC (Biolegend) | FACS | 102506 | 1 | ul/10⁶ cells |
| Anti- mouse CD31 PERCP-CY5.5 | FACS | 102522 | 1 | ul/10⁶ cells |
| (Biolegend) | | | | |
| Anti- mouse CD326 Percp-Cy5.5 (Ep-CAM) | FACS | 118220 | 1 | ul/10⁶ cells |
| (Biolegend) | | | | |
| Anti- mouse CD326 APC-Cy7 (Ep-CAM) | FACS | 118218 | 1 | ul/10⁶ cells |
| (Biolegend) | | | | |
| Anti- mouse CD326 APC (Ep-CAM) | FACS | 118213 | 1 | ul/10⁶ cells |
| (Biolegend) | | | | |
| Anti- mouse CD326 FITC (Ep-CAM) | FACS | 118207 | 1 | ul/10⁶ cells |
| (Biolegend) | | | | |
| Anti- mouse CD326 PE (Ep-CAM) | FACS | 118205 | 1 | ul/10⁶ cells |
| (Biolegend) | | | | |
| Anti-mouse TER-119/Erythroid Cell Pacific | FACS | 116232 | 1 | ul/10⁶ cells |
| Blue (Biolegend) | | | | |
| Anti-mouse TER-119/Erythroid Cell APC- | FACS | 116223 | 1 | ul/10⁶ cells |
| Cy7 (Biolegend) | | | | |
| Anti-mouse TER-119/Erythroid Cell APC | FACS | 116212 | 1 | ul/10⁶ cells |
| (Biolegend) | | | | |
| Anti-mouse CD117 (c-kit) PE (Biolegend) | FACS | 105808 | 1 | ul/10⁶ cells |
| SYTOX ™ Blue Dead Cell Stain | FACS | S34857 | 1 | uM |
| 7-AAD (BD Pharmingen) | FACS | 51-68981E | 1 | ul/10⁶ cells |
| Anti-mouse CD117 (c-kit) PE (Biolegend) | FACS | 105808 | 1 | ul/10⁶ cells |

| | | | |
|---|---|---|---|
| Secondary antibodies* | | | |
| Anti- chicken Alexa Fluor 488 | IHC | 703-545-155 | 1:200 |
| Anti- rabbit Rhodamine Red | IHC | 711-295-152 | 1:200 |
| Anti- rabbit Alexa Fluor 647 | IHC | 711-605-152 | 1:200 |
| Anti- rat Alexa Fluor 594 | IHC | 712-585-150 | 1:200 |
| Anti- goat Alexa Fluor 488 | IHC | 705-545-003 | 1:200 |
| Anti- goat Alexa Fluor 594 | IHC | 705-585-003 | 1:200 |
| Anti- goat AMCA | IHC | 705-155-003 | 1:200 |
| Anti- chicken Alexa Fluor 488 | IHC | 703-545-155 | 1:200 |
| Anti- rabbit Rhodamine Red | IHC | 711-295-152 | 1:200 |
| Anti- rabbit Alexa Fluor 647 | IHC | 711-605-152 | 1:200 |
| Anti- rat Alexa Fluor 594 | IHC | 712-585-150 | 1:200 |
| Anti- goat Alexa Fluor 488 | IHC | 705-545-003 | 1:200 |
| Anti- goat Alexa Fluor 594 | IHC | 705-585-003 | 1:200 |
| Anti- goat AMCA | IHC | 705-155-003 | 1:200 |
| DyLight ™ 405 AffiniPure Donkey Anti- | IHC | 705-475-003 | 1:100 |
| Goat IgG (H + L) | | | |
| Alexa Fluor ® 647 AffiniPure Donkey Anti- | IHC | 705-605-003 | 1:200 |
| Goat IgG (H + L) | | | |
| DyLight ™ 405 AffiniPure Donkey Anti- | IHC | 706-475-003 | 1:200 |
| Guinea Pig IgG (H + L) | | | |
| DyLight ™ 405 AffiniPure Donkey Anti- | IHC | 711-475-152 | 1:100 |
| Rabbit IgG (H + L) | | | |
| DyLight ™ 405 AffiniPure Donkey Anti-Rat | IHC | 712-475-150 | 1:100 |
| IgG (H + L) | | | |

*All the secondary antibodies were produced in donkey and were purchased from Jackson ImmunoResearch or Abcam (unless otherwise indicated).
*Detailed information about the antibodies, their specificity, cross-reactivity, application and isotype controls is available on the manufacturers' websites.

### Example 1

Identification of a Novel Population of Pulmonary Progenitor Cells which Dually Express Endothelial and Epitehlial Markers Long Term Chimerism in Recipient Lungs after Transplantation of Td-Tomato Labeled Cells

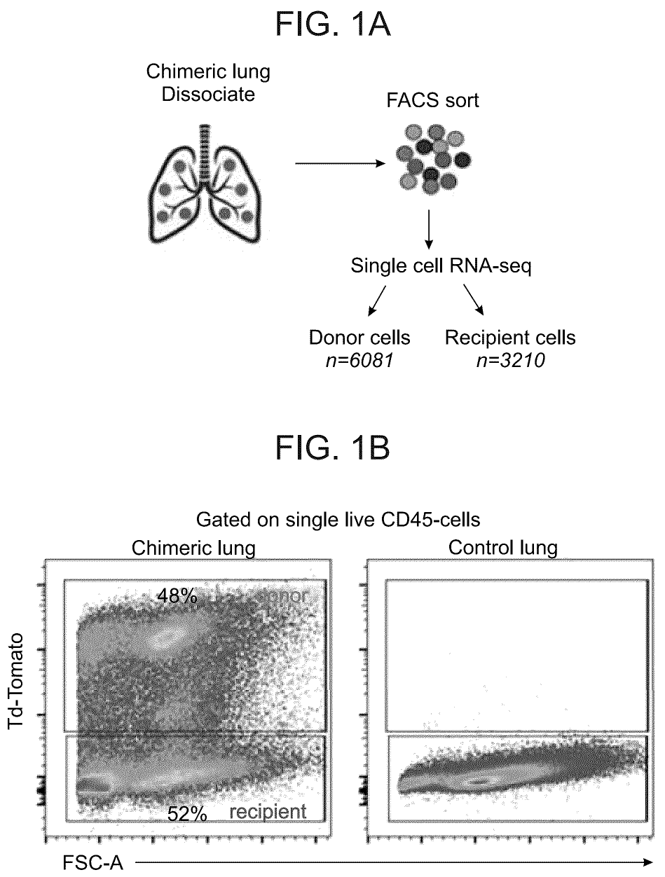

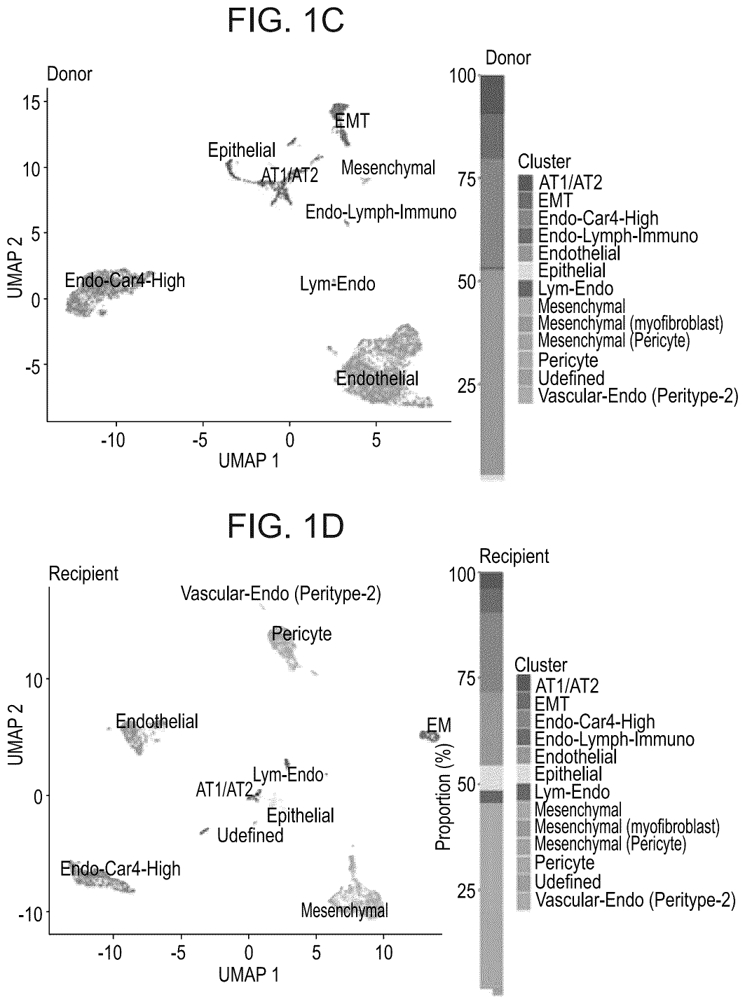

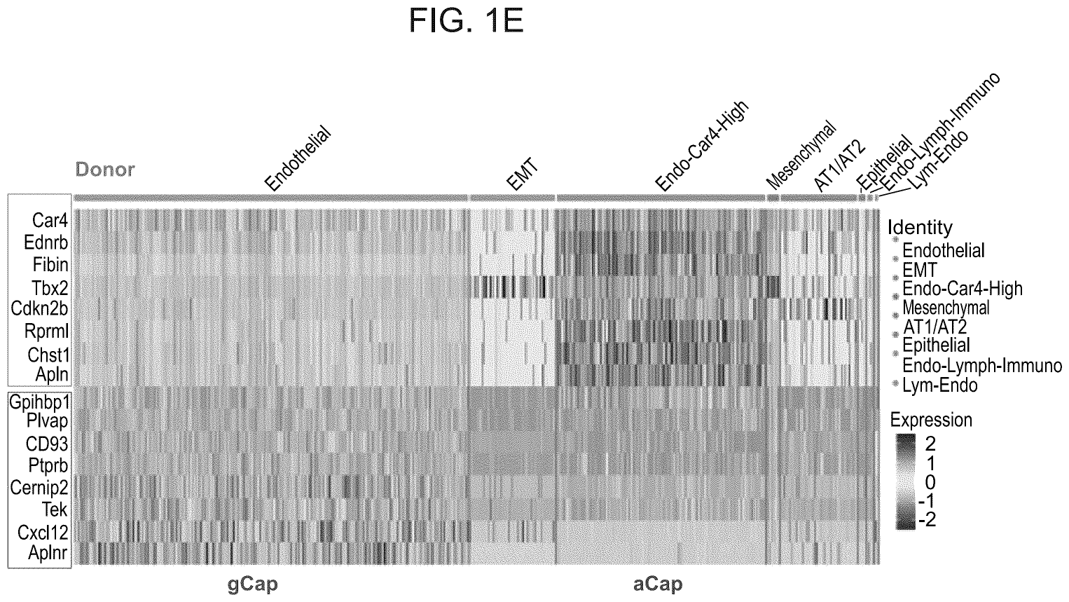

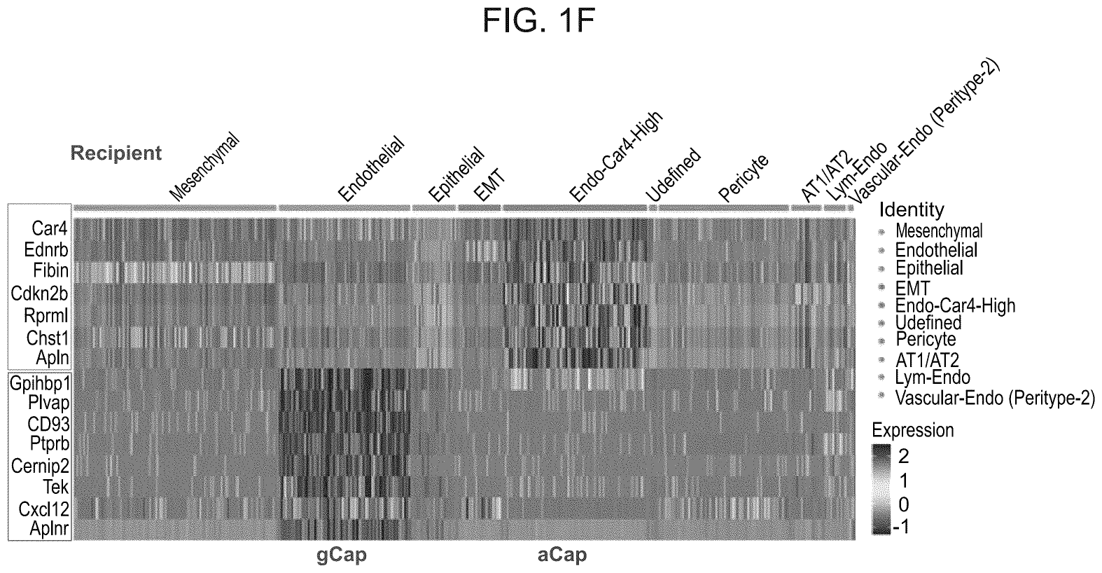

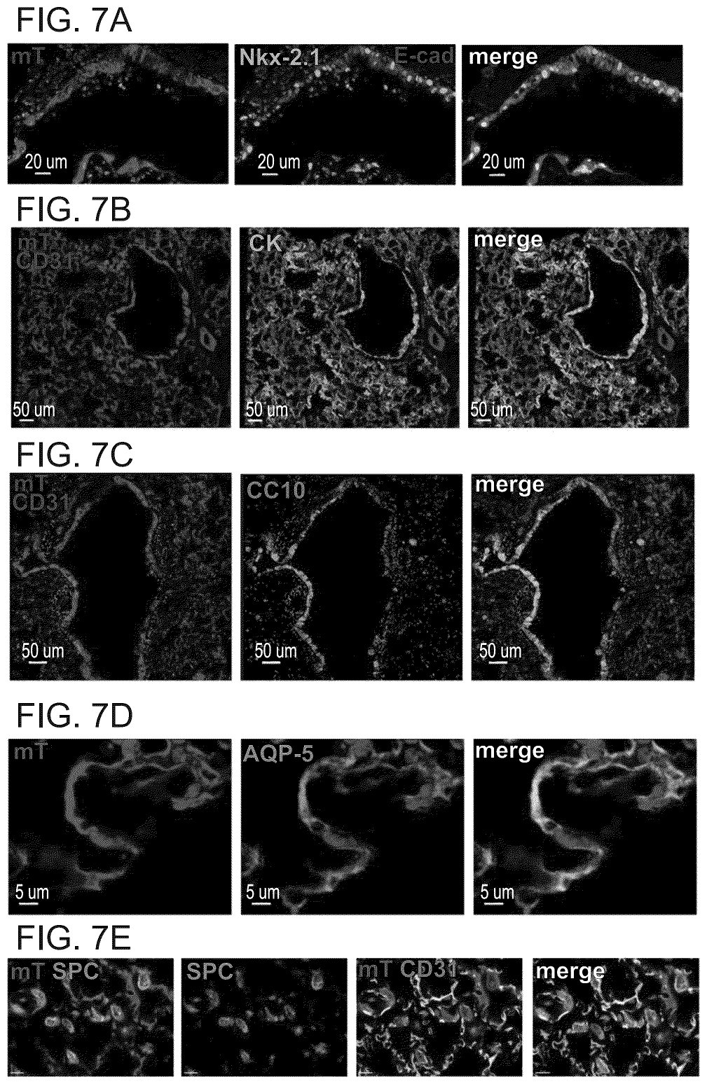

In previous studies immuno-histology (IH) was used to follow lung chimerism up to 4 months. A 6-8 months follow up of chimerism was performed using and combining results of immunohistology (FIGS. 7A-E) with single cell RNA transcriptome analysis (FIGS. 1A-F), showing donor derived epithelial (ciliated cells, club cells, AT1 and AT2 cells) and endothelial clusters (lymphatics, endothelial progenitors, as well as the newly described (10) gCap-“general”, and aCap-“aerocytes” capillary cells). The resulting clusters were defined based on marker genes and gene set analysis results, using publicaly available LGEA (Lung Gene Expression Analysis) Web Portal. For definition of each cluster we used well defined hallmark gene sets.

Transplantation of Lung Progenitors from Fetal or Adult Cag-Cre ER2 ‘Confetti’ Mice Results in Monochromatic Patches.

To unequivocally establish the single cell origin of the lung patches observed following transplantation, a set of experiments was carried out making use of the multicolor reporter system ‘R26R-Confetti’ mice (9) as donors. In these mice the recombination process is independent in each cell and stochastic. Daughter cells will produce the same fluorescent protein, and if indeed all the cells within each patch are derived from a single cell, they should all be of the same color. Multiple studies used this system to demonstrate single cell derived clonal behavior in organs such as intestine (9), (11), lung (12), brain (13), kidney, mammary gland, and others (14), (15), (16), (17), (18).

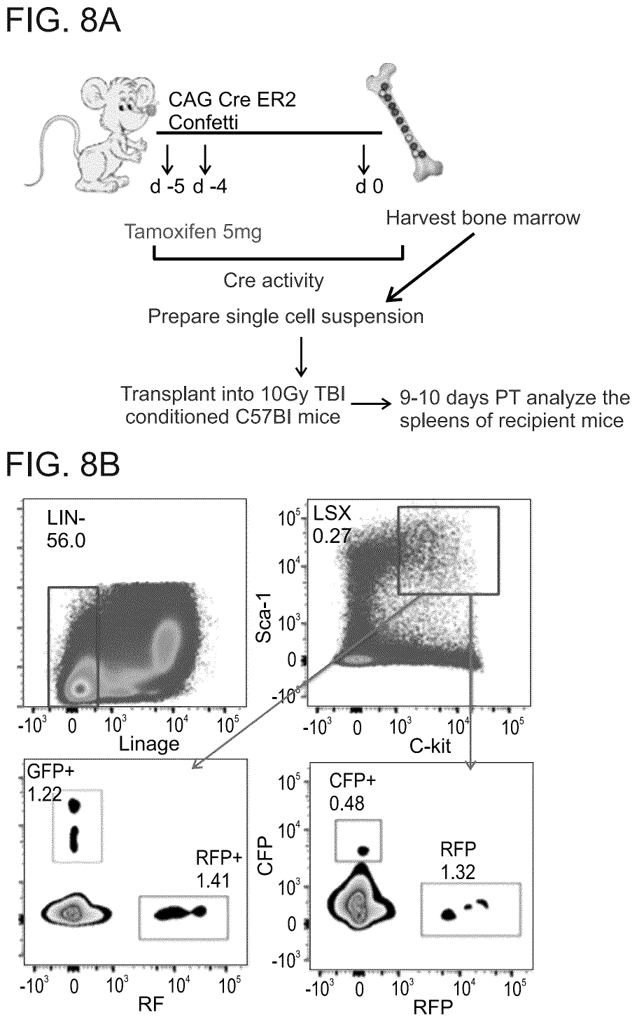

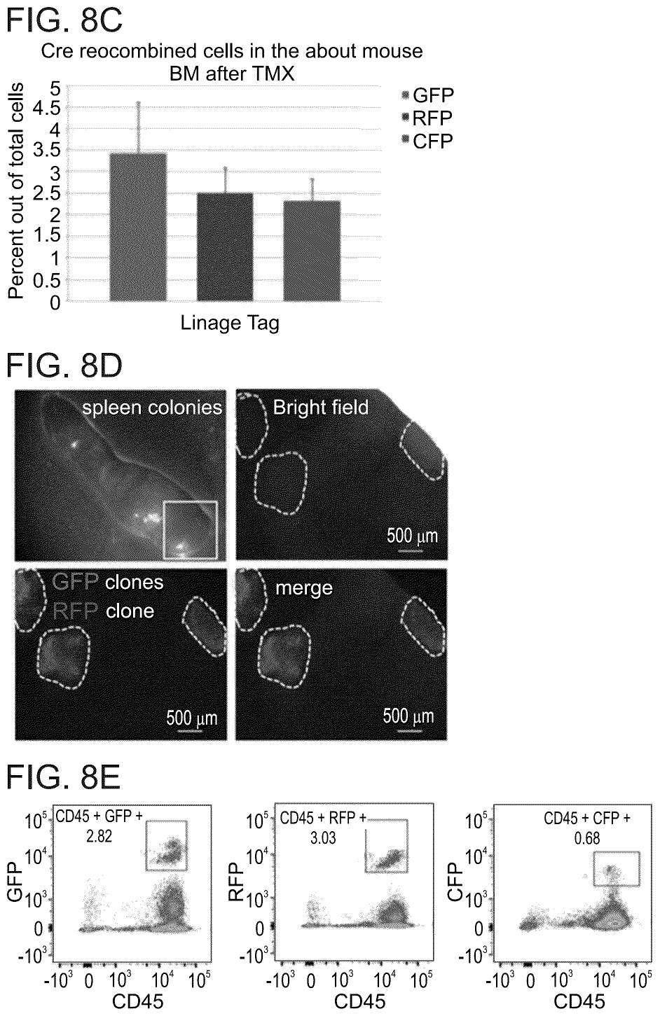

To verify the relevance of this approach to lung cell transplantation studies, the present inventors initially tested it in the context of hematopoietic stem cell transplantation known to yield single cell-derived spleen colonies (FIGS. 8A-E).

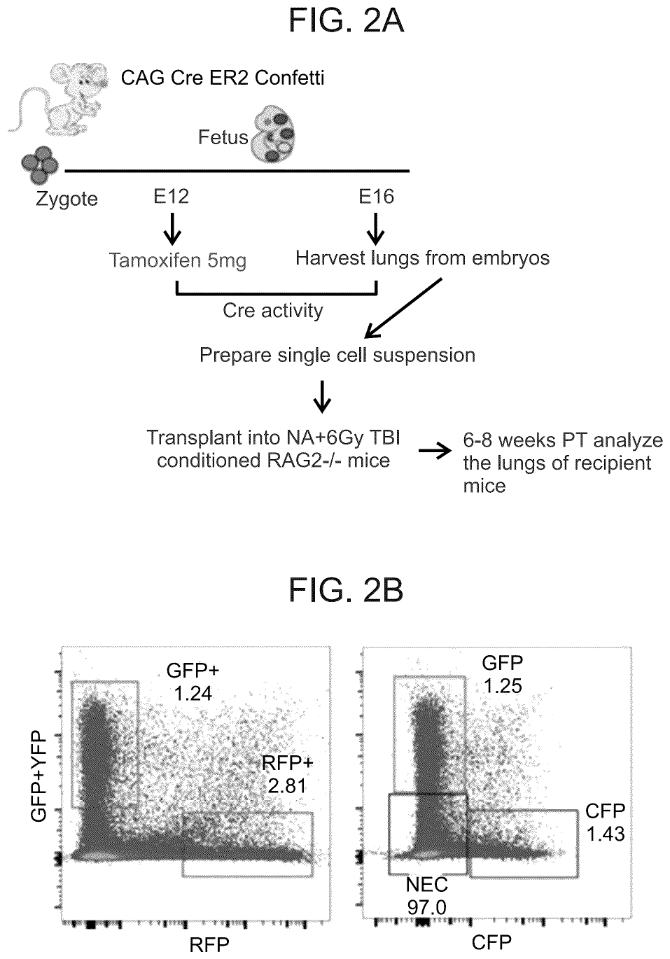

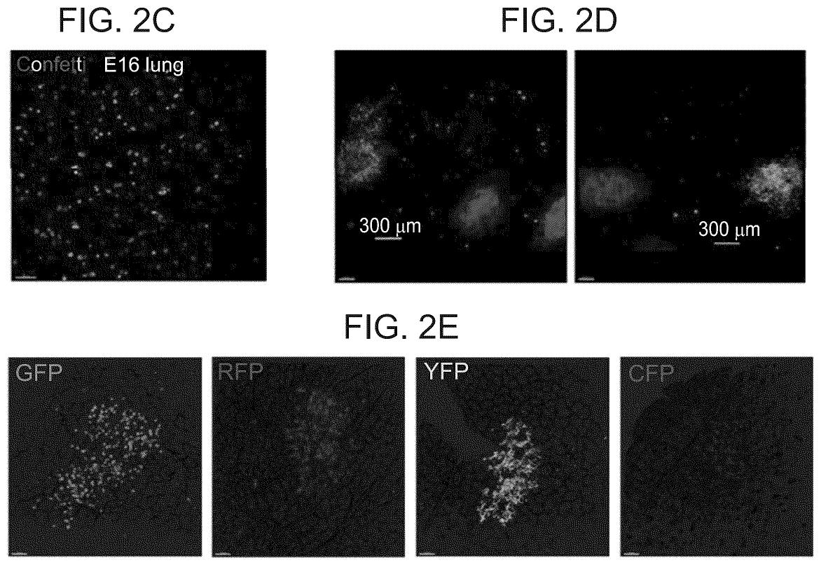

Next, lung patch formation was investigated following transplantation of lung cell suspension from E16 R26R-Confetti donors into RAG-2 recipients preconditioned with naphthalene and 6Gy TBI (see schematic presentation in FIG. 2A). The embryos were harvested at E16, Tamoxifen (TMX) was administered to pregnant females at E12, and the fetal lungs, expressing fluorescent cells were isolated under a fluorescent microscope. Approximately 5-6% of the harvested lungs expressed one of the fluorescent tags (FIG. 2B). Two photon micrographs depict typical monochromatic cells prior to transplantation, each expressing one of the four fluorescent tags (FIG. 2C).

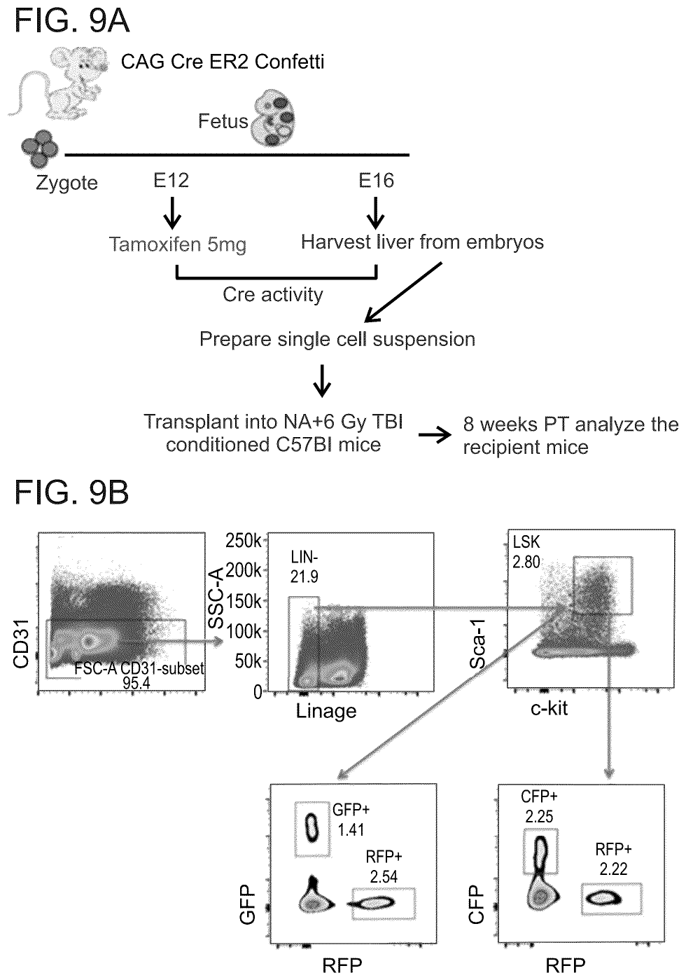

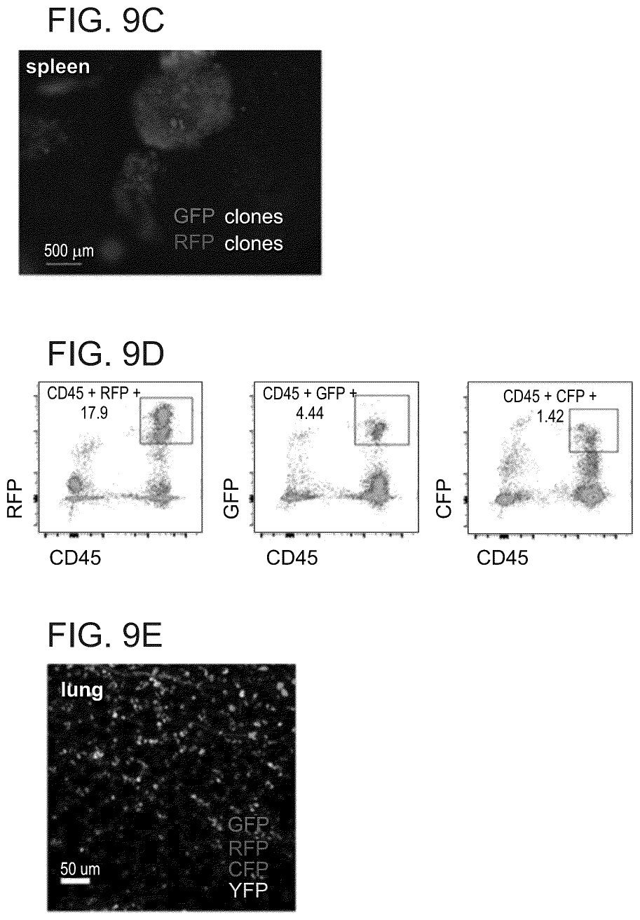

Transplantation of fetal lung cells from Tamoxifen-induced donors into naphthalene treated and irradiated recipient mice, resulted in discrete monochromatic lung patches, expressing one of the four fluorescent proteins, strongly indicating that each patch is likely derived from a single lung progenitor (FIGS. 2D-E). Similar transplantation of R26R-Confetti fetal liver single cell suspension (shown schematically in FIG. 9A), expressing the fluorescent tags in hematopoietic LSK+ (Linage-Scal+c-kit+) stem cells (FIG. 9B) inducing hematopoietic spleen colonies (FIG. 9C) as well as hematopoietic blood chimerism (FIG. 9D), failed to produce donor-derived lung patches (FIG. 9E), strongly supporting the lung specificity of the observed patch-forming activity.

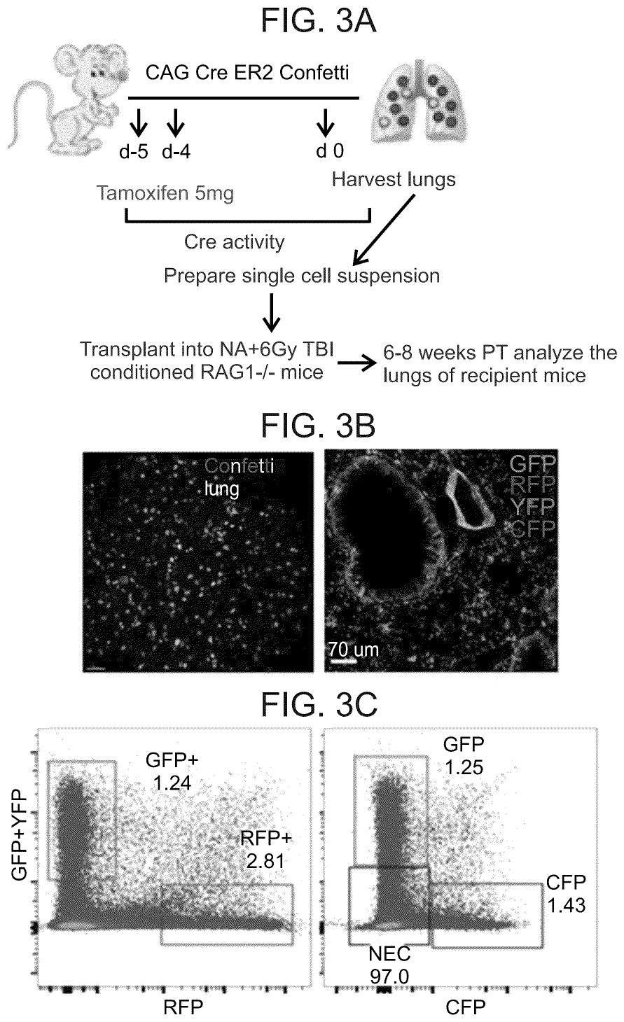

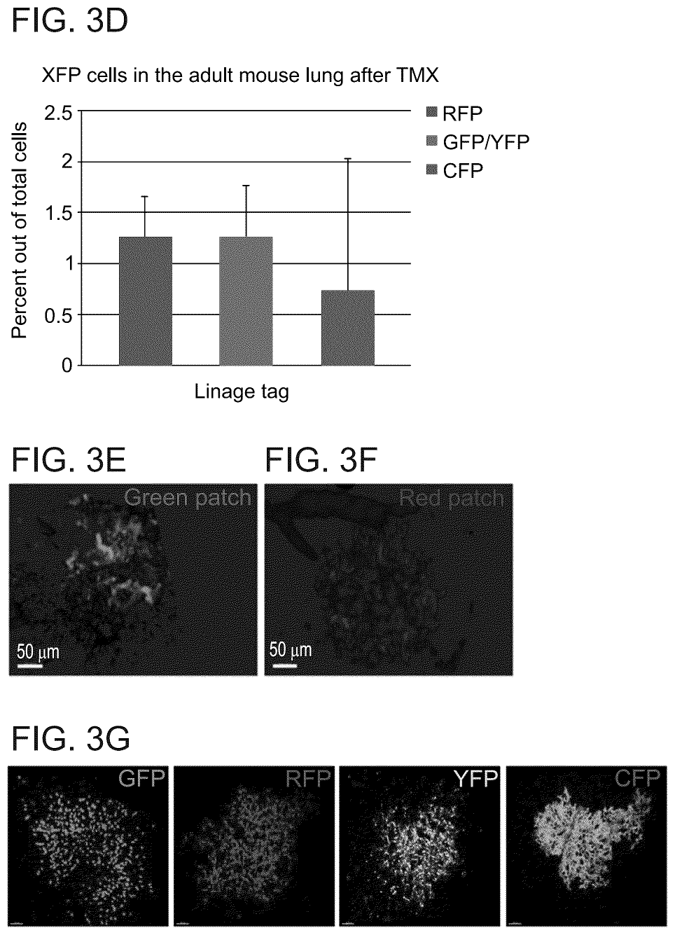

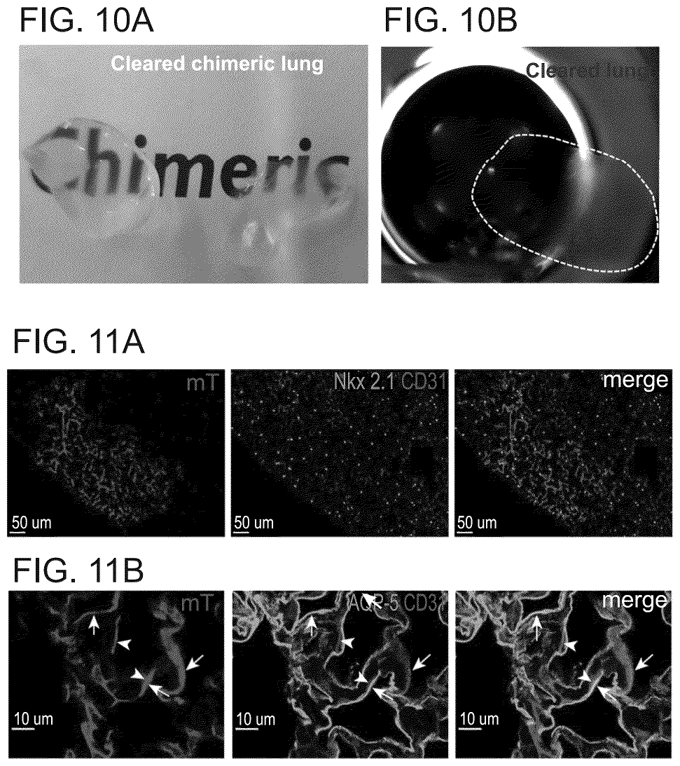

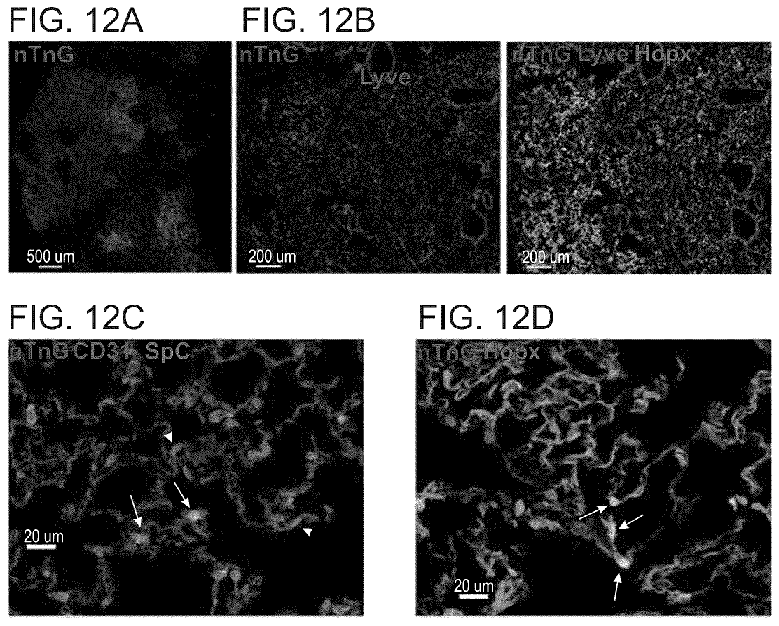

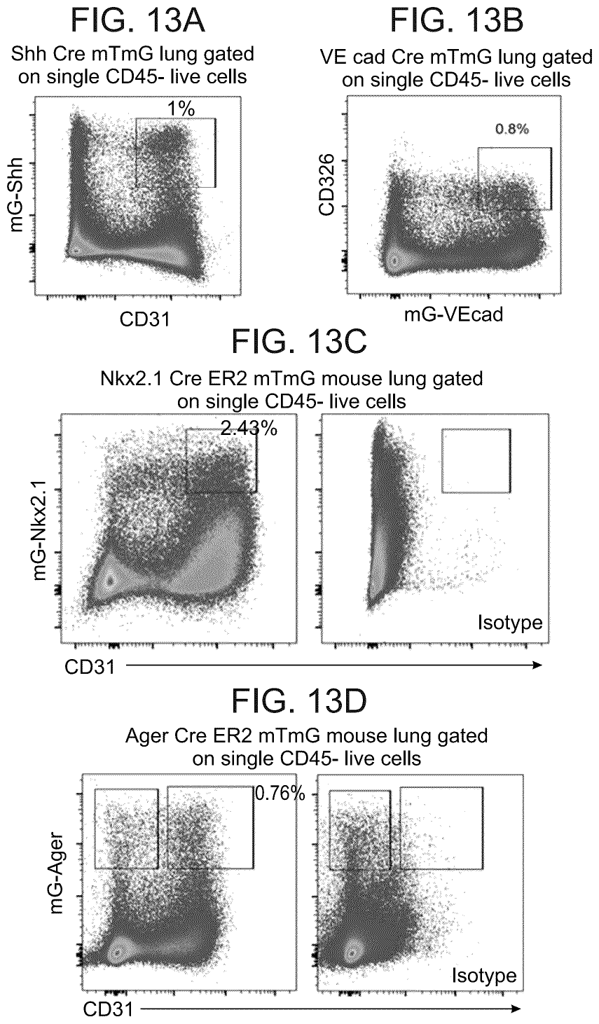

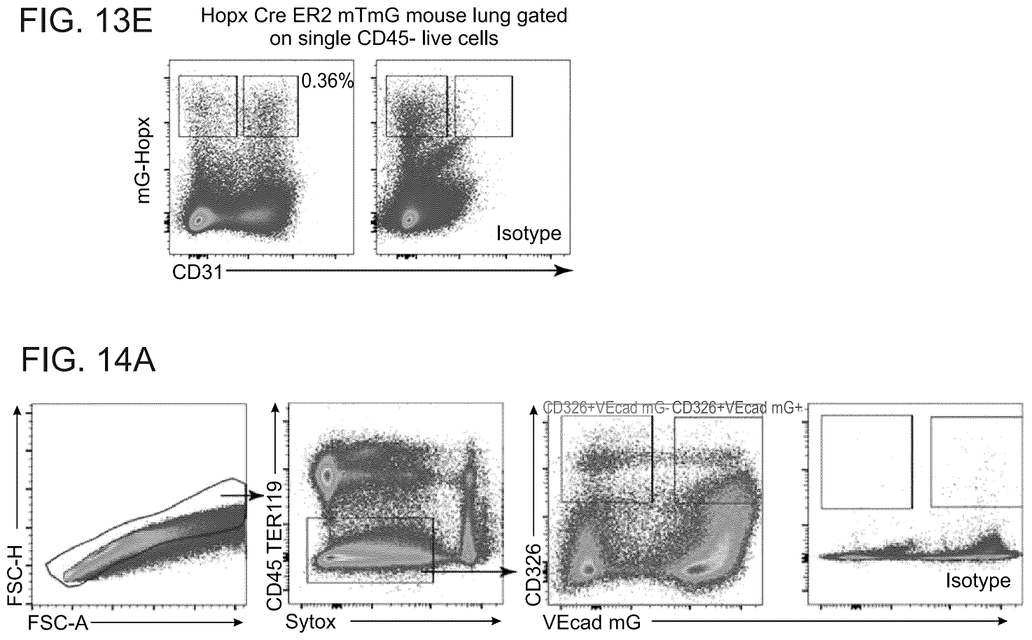

Next, based on recent finding that patch forming progenitor cells are also present in the adult mouse lung, the same approach was applied to analyze the origin of lung patches after transplantation of adult lung cells. To that end, the protocol described above was used, but since the frequency of patch forming cells in the adult lung is about 3-4 fold lower compared to that found in E16 fetal lungs (7), and only a fraction of the cells undergo the Cre recombination, a higher dose (16×10⁶) of lung cells was used for transplantation (FIG. 3A). As shown in FIG. 3B, following TMX treatment, monochromatic GFP, YFP, RFP and CFP fluorescent cells could be documented by two photon microscopy within the adult lung, and as shown by confocal microscopy these tagged cells were distributed throughout the entire lung (FIG. 3B). Further FACS analysis of the R26R-Confetti adult lung, after 2 doses of TMX, allowed detecting the percentage of Cre recombined cells (up to 5%; FIGS. 3C-D). To evaluate the clonality of donor-derived patches at 8 weeks after transplantation, confocal, two photon, and light sheet microscopy were used. As shown in FIGS. 3E-F, and FIG. 10A-B, light sheet microscopy of “cleared” chimeric lungs revealed distinct monochromatic patches.

Likewise, two photon snapshots of whole mount chimeric lung documented monochromatic patches expressing cither membranous CFP, nuclear GFP, or cytoplasmatic RFP or YFP (FIG. 3G). The full depth 2-photon microscopy scan of these monochromatic patches in the whole mount lung tissue was presented (data not shown).

Altogether, a total of 50 fields collected from 15 chimeric mice, including 12 transplanted with adult lung cells, and 3 with E16 fetal lung cells were analyzed; and notably, an exclusive presence of monochromatic donor-derived patches was found. Testing the experimental distribution of single-color (n=50) vs. two-color (n=0) clones, against the theoretical distribution of 25% vs. 75% (X2₁=150), by X2 distribution test suggests that it is highly unlikely that any clone is derived from two cells (p<0.001), demonstrating that the lung patches observed after transplantation of fetal or adult lung cells are derived from a single progenitor cell.

### Two Distinct Patch-Forming Lung Cell Progenitors.

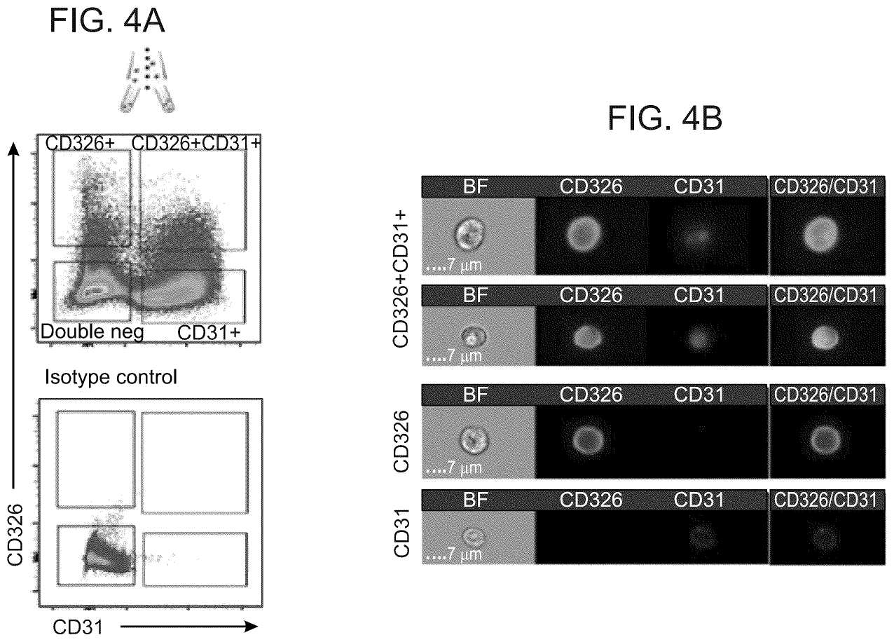

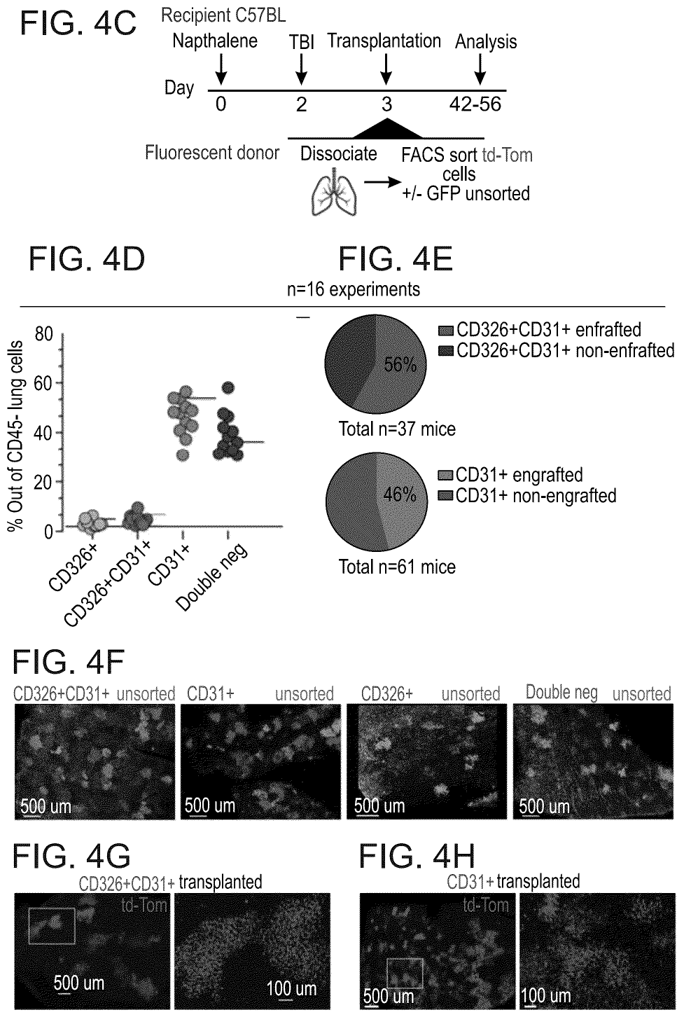

To define the identity of the putative “patch-forming” cell in the system, FACS purified cell populations from the mouse lung were transplanted. To this end, labeled donor mice, including GFP (C57BL/6-Tg (CAG-EGFP) \Osb/J), mTmG (Gt(ROSA)26Sor^{tm4(ACTB-tdTomato,-EGFP)Lao}/J). and nTnG (Gt(ROSA)26Sor^{tm1(CAG-tdTomuto*,-EGFP*)Ees}/J) mice, (mTmG mice express membrane td-tomato, and nTnG express nuclear td-tomato) were used. The unique localization of the fluorescent tag either in the membrane or the nuclei of the sorted lung cells enabled tracking membranous, cytoplasmatic and nuclear epithelial and endothelial markers at the single cell level within the monochromatic patches of chimeric lungs and to characterize the cellular composition of the patches after transplantation of the sorted lung cell subpopulations. In line with the observation that large proportion of the patches contain both endothelial and epithelial cells, and that each such a patch is derived from a single progenitor, a putative multipotent lung progenitor capable of differentiating along these two distinct lineages was searched for. FACS analysis (FIG. 4A) as well as image-stream analysis (FIG. 4B) of CD45 negative lung cells, revealed a unique subpopulation expressing both the CD326 epithelial marker and the CD31 endothelial marker. Thus, it was hypothesized that the patch-forming cells might be included within this double positive subpopulation. To test this hypothesis, the transplantation assay for patch forming cells was used after sorting of the four lung subpopulations obtained upon staining for CD326 and CD31, as described in FIG. 4C.

Patch forming activity could be found only upon transplantation of CD326+CD31+ double positive cells (21 of 37 mice) or single positive CD326-CD31+ cells (28 out of 61 mice), while no patch-forming activity was found in the single positive CD326+CD31-epithelial cell fraction or in the double negative CD326-CD31-fraction (FIG. 4F). It is possible that sorted cells might need other facilitating cells in the donor cell preparation for the early steps of colonization in the recipient lungs. It was therefore attempted to transplant 0.3-0.5×10⁶ sorted cells from lungs of mTmG or nTnG mice, with (FIG. 4F) or without (FIGS. 4G-H) 0.5×10⁶ unsorted lung cells from GFP+ donors. These experiments clearly demonstrated that transplanted FACS sorted cells are capable of forming patches even in the absence of potential supporting cells from the co-transplanted unseparated GFP+ lung cell preparation (FIGS. 4G-H).

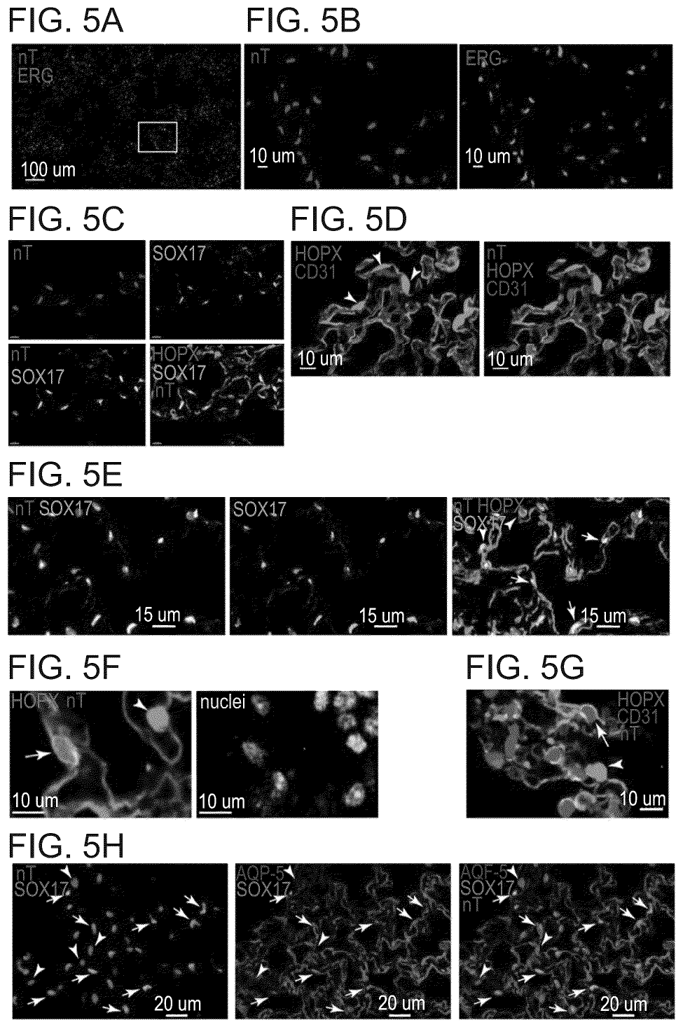

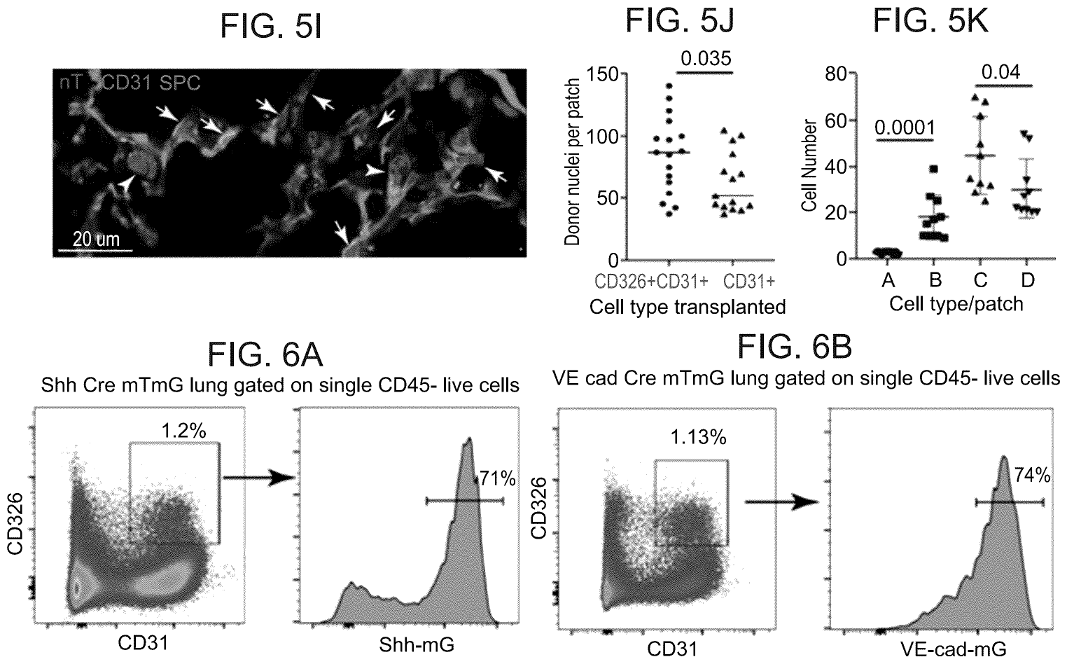

Staining of the donor-derived patches for epithelial alveolar markers including AQP-5, HOPX and SPC, or for endothelial markers including CD31, ERG and SOX17, demonstrated that the patches formed following transplantation of the double positive subpopulation contained both endothelial and epithelial cells (FIGS. 5E-I, 11A-B and 12A-D), while patches formed by the sorted CD326-CD31+ single positive cells were comprised predominantly of endothelial cells (FIGS. 5A-D). This analysis is strongly supported by the use of nuclear staining allowing distinction of cellular borders between neighboring cells. To this end, colocalization of the donor-derived nT marker with nuclear ERG/SOX17 or with nuclear expression of HOPX, enabled identifying donor-derived endothelial or AT1 epithelial cells (FIGS. 5E-G). Likewise, AT1 cells stained for AQP-5 could be distinguished within the patches from endothelial cells stained for SOX17 (FIG. 5H). A similar distinction of AT2 cells from endothelial cells could be attained by staining for CD31 and smRNA FISH probe for SPC (FIG. 5I). Quantitative analysis of the cell numbers comprising the patches suggests that those found after transplantation of CD326+CD31+ lung cells were larger than those found after transplantation of CD326-CD31+ cells (FIG. 5J). This analysis confirmed the difference in cellular composition between the two types of patches, with co-localization of endothelial and epithelial cells mostly found in the patches formed following transplantation of CD326+CD31+ lung cells (FIG. 5K).

### Further Characterization of the CD326+CD31+ Patch Forming Lung Progenitors

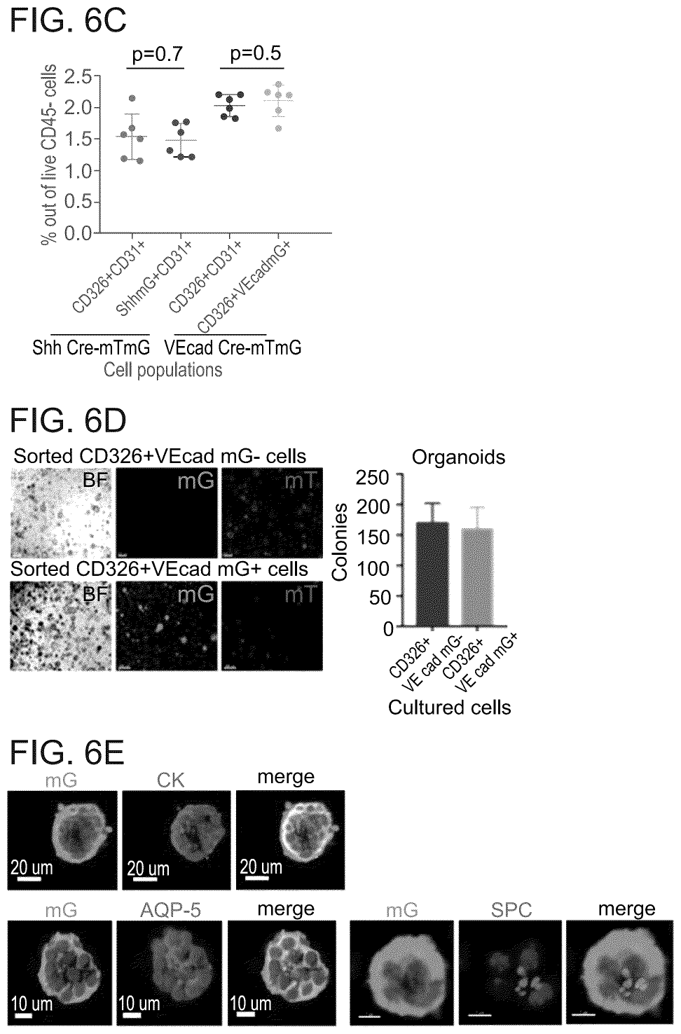

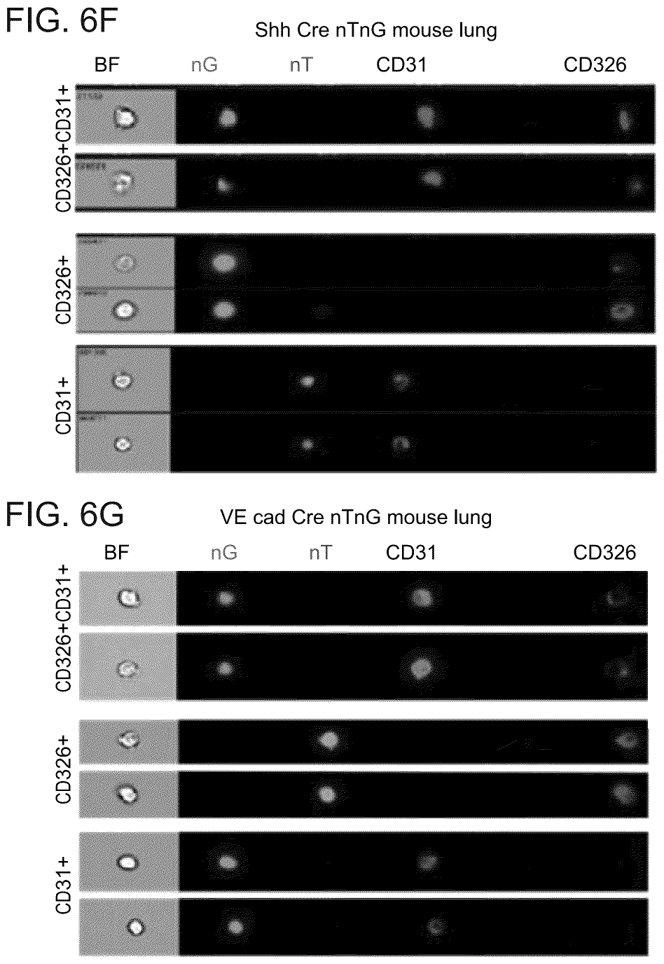

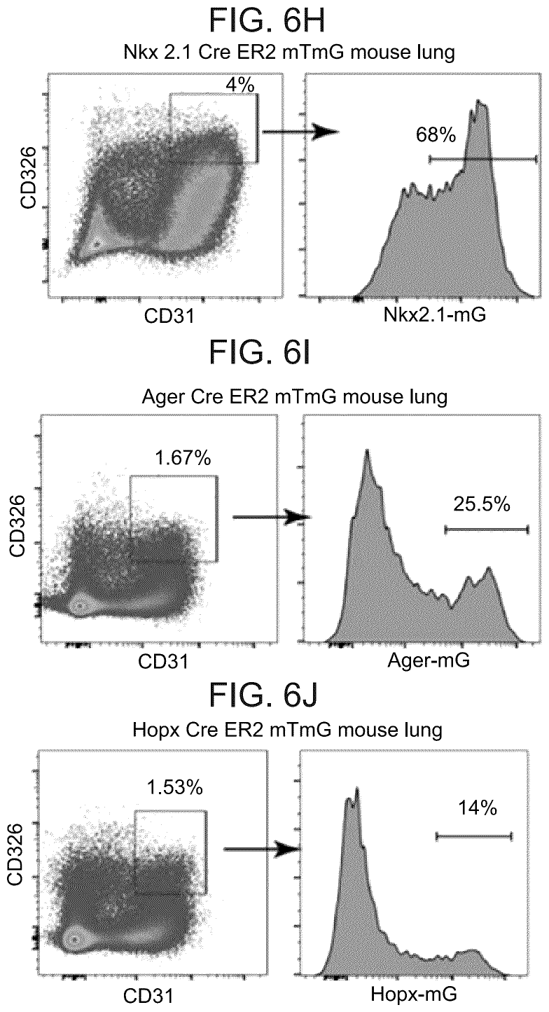

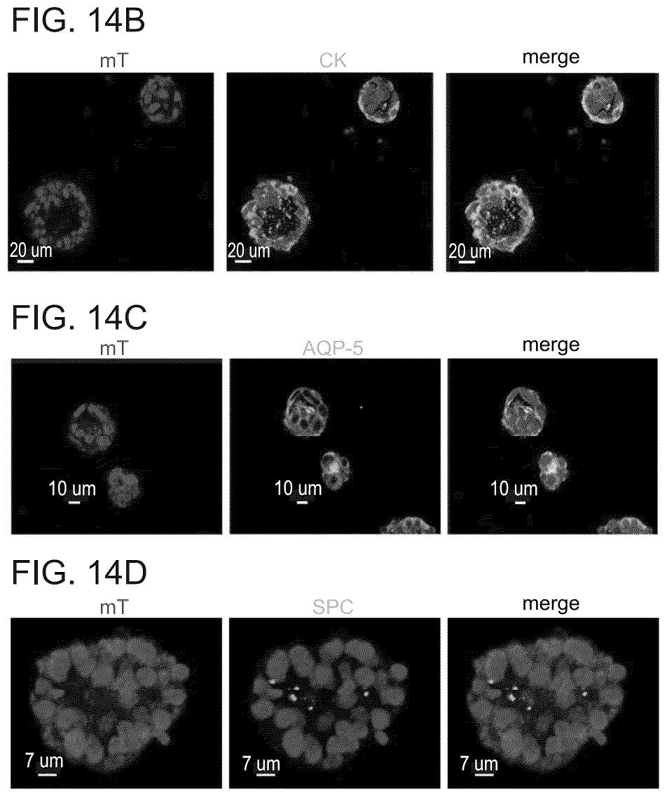

To further characterize the sorted CD326+CD31+ lung subpopulations, transgenic mice expressing GFP under different promoters which are typically activated in epithelial or endothelial cells, such as Sonic hedgehog (Shh) and VE-cadherin (Cadherin 5) were used. Shh Cre mTmG and Shh Cre nTnG mice (generated by breeding of B6.Cg-Shh^{tm1(EGFP/cre)Cjt}/J with Gt(ROSA)26Sor^{tm4(ACTB-tdTomato EGFP)Luo}/J and B6N.129S6-Gt (ROSA) 26Sor^{tm1(CAG-tdTomato*-EGFP*)Ees}/J mice, respectively) express GFP in the epithelial lineage (19, 20), while VE cadherin Cre mTmG and VE cadherin Cre nTnG mice (generated by breeding of B6.129-Tg (Cdh5-cre) 1Spc/Jmice with Gt (ROSA) 26Sortm4 (ACTB-tdTomato,-EGFP) Luo/J and B6N.129S6-Gt (ROSA)26Sor^{tm1(CAG-tdTomato*,-EGFP*)Ees}/J mice, respectively) express GFP in the endothelial lineage (21). FACS analysis of the lungs of these transgenic mice revealed that double positive CD326+CD31+ lung progenitor cells also express additional typical endothelial and epithelial markers, namely VE-cadherin (71%) and Shh (74%) (FIGS. 6A-C). Furthermore, apart from the ability of the CD326+CD31+ progenitors cells to form single cell derived patches comprised of donor derived epithelial and endothelial cells, these cells can generate epithelial organoids in-vitro and using this assay it was confirmed that VE-cadherin+ cells within the CD326+CD31+ subpopulation purified from the lungs of VE cadherin Cre mTmG mouse donors, indeed are capable of forming epithelial colonies. Thus, the organoids generated from FACS purified double positive CD326+VEcad mG+ cells exhibited GFP+ epithelial cells, in contrast to the organoids generated from sorted CD326+VEcad mG-lung cells, which are negative for GFP and express mT (FIGS. 6D and 14A-D). The epithelial character of the GFP+ organoids was confirmed by their staining for additional epithelial markers, such as cytokeratin, AQP-5 and SPC (FIG. 6E).

The duality of the CD326+CD31+ cells was also confirmed by Imagestream analysis of individual cells. Thus, CD326+CD31+ lung cell progenitors from Shh Cre nTnG mice or VEcad Cre nTnG mice were found to be also double positive for Shh and VE cad (FIGS. 6F-G and 13A-E). Furthermore, analysis of lungs from Nkx 2.1CreER2 mTmG (22), in which Cre recombination was induced by administration of single dose of Tamoxifen, 6 days prior to FACS analysis, showed similarly high level of NKX2.1 (68%) within the CD31+CD326+ lung cell population. Notably, Ager-CreER2 mTmG (23), (24) and Hopx-CreER2 mTmG transgenic mice (25), (26), (27), showed substantial levels of RAGE-receptor for advanced glycation end products (Ager) (25.5%) and lower expression of Homeobox only protein x (Hopx) (14%). Taken together, these results, which are based on lineage-specific GFP expression in transgenic mice as opposed to cell surface antibody staining, further support the dual character of the CD326+CD31+ lung progenitors (FIGS. 6H-J).

### DISCUSSION

It was recently demonstrated that 6-8 weeks after transplantation of mouse or human fetal lung cells into mice preconditioned with naphthalene and 6Gy TBI, numerous donor-derived patches comprising epithelial and endothelial cells could be detected throughout the lung (6). Similar findings were subsequently demonstrated following transplantation of adult lung cells, though the concentration of patch forming cells was 3-4 fold lower compared to that in the fetal lung (7). The present inventors have now found using R26R-Confetti donors, in which each fluorescent cell is labelled in one of 4 potential colors, by virtue of Cre-recombination that each donor derived lung patch found after transplantation of fetal or adult mouse lung cells is formed by colonization and differentiation of a single mutli-potent lung progenitor. Furthermore, this finding enabled characterizing the putative multi-potent lung progenitor by FACS purification. Thus, using the transplantation assay, two distinct patch forming progenitors, namely, CD326+CD31+ and CD326-CD31+ cells were found within the non-hematopoietic CD45 negative lung compartment. However only the former was capable of forming patches comprising both epithelial and endothelial cells while the latter was predominantly associated with the formation of endothelial cells.

A classic example of such cellular plasticity was demonstrated by Tata et al. (28, 29), showing de-differentiation of fully mature secretory cells into basal stem cells with regenerative capacity. Another example of cellular plasticity is known as transdifferentiation or transdetermination, when stem cells from one region of the lung can convert into stem cells of the other lung region (29). Furthermore, evidence for multi-potential progenitors capable of developing into endothelial and epithelial linages, has also been described for human breast progenitors, which were found to express a significant level of ‘Yamanaka’ transcription factors (30, 31).

Notably, the dual character of the double positive CD326+CD31+ progenitors was further substantiated by the demonstration that in transgenic mice expressing fluorescent tags under the Shh or VE-cadherin promoters, the double positive CD326+CD31+ cells also express both these typical epithelial and endothelial markers. In addition, using the same approach with tamoxifen induced Cre-recombination it was found that the CD326+CD31+ cell fraction also comprises similar levels of NKX2.1 and significant although lower levels of cells expressing Ager and Hopx. The observed expression of NKX2.1 is in line with previous suggestions that this transcription factor which is a hallmark of pulmonary specification is expressed in endothelial cells in the developing lung (32). Notably, Ager a multiligand pattern recognition receptor implicated in several disease states, is extensively expressed on AT1 cells, and on alveolar endothelium as well (24). Hopx (Homeobox only protein x) is an AT1 marker and is an important regulator of cardiac development (33) (34) and of hair follicle, intestinal and hematopoetic stem cell biology (35), (36), (37), (38).

In conclusion, the data reveal a novel lung subpopulation comprising multi-potent CD326+CD31+ lung progenitors capable of lung injury repair. While different lung progenitors restricted to differentiation along epithelial lineages have been described before, this unique progenitor exhibits a dual phenotype expressing well-established epithelial and endothelial markers, and can differentiate into both epithelial or endothelial fates following transplantation into lung-injured mice. This duality is of particular value for lung injury repair considering that all the major lung diseases exhibit not only epithelial injuries, but are also associated with endothelial damage.

Furthermore, apart from its potential for translational studies aiming at the correction of different lung diseases, the identification of lung progenitors could also contribute to basic studies aiming at better understanding of fetal lung development as well as of steady state maintenance of different cellular lineages in the adult lung.

### Example 2

Expansion of the Pulmonary Progenitor Cells which Dually

### Express Endothelial and Epitehlial Markers in Culture

Considering that double positive CD31+CD326+ lung cells represent the patch forming lung progenitors, this marker was used to guide the ex-vivo expansion strategy. Briefly, lungs were freshly harvested from 6-12 weeks old C57BL/6 mice enzymatically digested into single cell suspension and depleted of CD45+ cells. Following, the cells were cultured in different media so as to optimize the most suitable conditions for expansion of CD31+CD326+ cells.

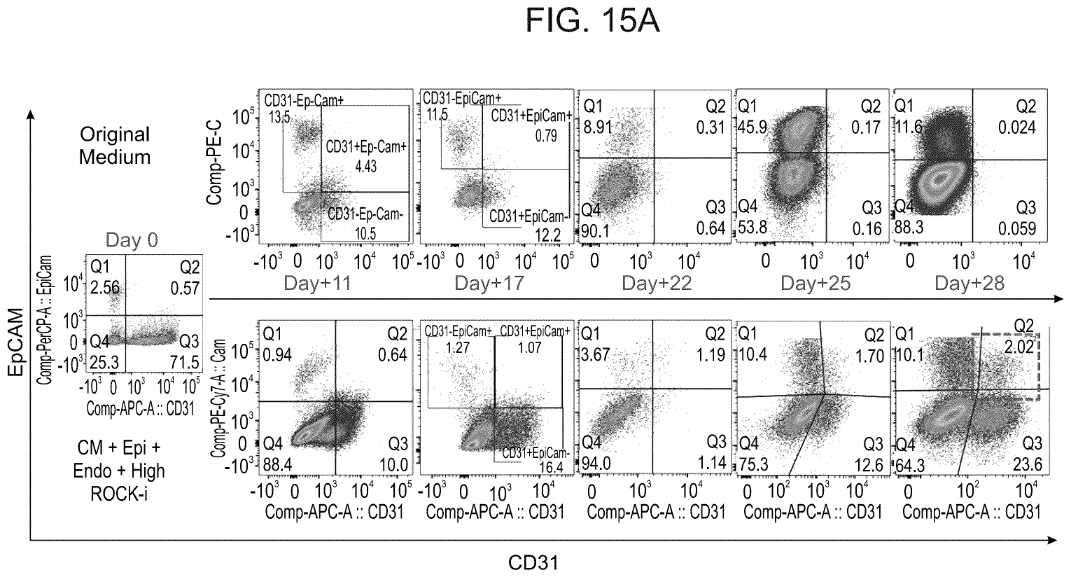

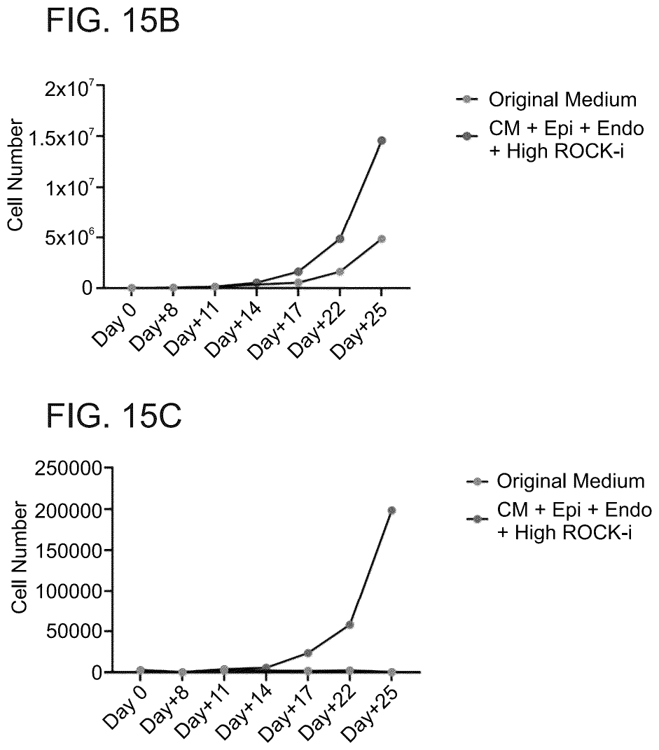

Following 4-5 passages it was found that culturing in a medium containing EGF with a low dose of a ROCK inhibitor resulted in expansion of the total number of cells but induced differentiation towards epithelial fate without retention of double positive CD31+CD326+ cells in the culture (FIGS. 15A-C). On the contrary, culturing in a medium comprising VEGF, EGF and a high does of a ROCK inhibitor led to a marked expansion of CD31+CD326+ cells (FIGS. 15A-C).

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

(other references are cited throughout the application)
1. D. J. Weiss, L. A. Ortiz, Cell therapy trials for lung diseases: progress and cautions. American Journal of Respiratory and Critical Care Medicine. 188, 123-125 (2013).
2. W. Zuo et al., p63 Krt5 distal airway stem cells are essential for lung regeneration. Nature. (2014).
3. D. J. Weiss et al., Stem cells and cell therapies in lung biology and lung diseases. Proceedings of the American Thoracic Society. 8, 223-272 (2011).
4. M. Nichane et al., Isolation and 3D expansion of multipotent Sox9 mouse lung progenitors. Nature Methods. 14, 1205 (2017).
5. Q. Ma et al., Regeneration of functional alveoli by adult human SOX9 airway basal cell transplantation. Protein & Cell. 9, 267-282 (2018).
6. C. Rosen et al., Preconditioning allows engraftment of mouse and human embryonic lung cells, enabling lung repair in mice. Nat. Med. 21, 869 (2015).
7. I. Milman Krentsis et al., Lung Injury Repair by Transplantation of Adult Lung Cells Following Preconditioning of Recipient Mice. Stem Cells Translational Medicine. 7, 68-77 (2018).
8. C. Hillel-Karniel et al., Multi-lineage Lung Regeneration by Stem Cell Transplantation across Major Genetic Barriers. Cell Reports. 30, 807-819. e4 (2020).
9. H. J. Snippert et al., Intestinal crypt homeostasis results from neutral competition between symmetrically dividing Lgr5 stem cells. Cell. 143, 134-144 (2010).
10. A. Gillich et al., Capillary cell-type specialization in the alveolus. Nature. 586, 785-789 (2020).
11. A. G. Schepers et al., Lineage tracing reveals Lgr5+ stem cell activity in mouse intestinal adenomas. Science. 337, 730-735 (2012).
12. T. J. Desai, D. G. Brownfield, M. A. Krasnow, Alveolar progenitor and stem cells in lung development, renewal and cancer. Nature. 507, 190-194 (2014).
13. A. Baggiolini et al., Premigratory and migratory neural crest cells are multipotent in vivo. Cell Stem Cell. 16, 314-322 (2015).
14. A. C. Rios, N. Y. Fu, G. J. Lindeman, J. E. Visvader, In situ identification of bipotent stem cells in the mammary gland. Nature. 506, 322 (2014).
15. C. L. Scheele et al., Identity and dynamics of mammary stem cells during branching morphogenesis. Nature. 542, 313 (2017).
16. P. R. Jamieson et al., Derivation of a robust mouse mammary organoid system for studying tissue dynamics. Development. 144, 1065-1071 (2017).
17. M. Ganuza et al., Lifelong haematopoiesis is established by hundreds of precursors throughout mammalian ontogeny. Nat. Cell Biol. 19, 1153 (2017).
18. E. Lazzeri et al., Endocycle-related tubular cell hypertrophy and progenitor proliferation recover renal function after acute kidney injury. Nature Communications. 9, 1344 (2018).
19. D. N. Watkins et al., Hedgehog signalling within airway epithelial progenitors and in small-cell lung cancer. Nature. 422, 313-317 (2003).
20. Y. Litingtung, L. Lei, H. Westphal, C. Chiang, Sonic hedgehog is essential to foregut development. Nat. Genet. 20, 58-61 (1998).
21. M. J. Chen, T. Yokomizo, B. M. Zeigler, E. Dzierzak, N. A. Speck, Runx1 is required for the endothelial to haematopoietic cell transition but not thereafter. Nature. 457, 887-891 (2009).
22. H. Taniguchi, J. Lu, Z. J. Huang, The spatial and temporal origin of chandelier cells in mouse neocortex. Science. 339, 70-74 (2013).
23. M. Chung, B. L. Hogan, Ager-CreER T2: a new genetic tool for studying lung alveolar development, homeostasis, and repair. American Journal of Respiratory Cell and Molecular Biology. 59, 706-712 (2018).
24. S. T. Buckley, C. Ehrhardt, The receptor for advanced glycation end products (RAGE) and the lung. Journal of Biomedicine and Biotechnology. 2010 (2010).
25. R. Jain et al., Plasticity of Hopx type I alveolar cells to regenerate type II cells in the lung. Nature Communications. 6, 1-11 (2015).
26. Z. Yin et al., Hop functions downstream of Nkx2. 1 and GATA6 to mediate HDAC-dependent negative regulation of pulmonary gene expression. American Journal of Physiology-Lung Cellular and Molecular Physiology. 291, L191-L199 (2006).
27. Y. Liu, W. Zhang, The role of HOPX in normal tissues and tumor progression. Biosci. Rep. 40 (2020).
28. P. R. Tata et al., Dedifferentiation of committed epithelial cells into stem cells in vivo. Nature. 503, 218 (2013).
29. P. R. Tata, J. Rajagopal, Plasticity in the lung: making and breaking cell identity. Development. 144, 755-766 (2017).
30. D. Pan et al., SOX2, OCT3/4 and NANOG expression and cellular plasticity in rare human somatic cells requires CD73. Cell. Signal. 28, 1923-1932 (2016).
31. S. Roy et al., Rare somatic cells from human breast tissue exhibit extensive lineage plasticity. Proc. Natl. Acad. Sci. U.S.A 110, 4598-4603 (2013).
32. K. I. Bostrom, J. Yao, X. Wu, Y. Yao, Endothelial Cells May Have Tissue-Specific Origins. J. Cell. Biol. Histol. 1 (2018).
33. R. Jain et al., HEART DEVELOPMENT. Integration of Bmp and Wnt signaling by Hopx specifies commitment of cardiomyoblasts. Science. 348, aaa6071 (2015).
34. C. M. Trivedi et al., Hopx and Hdac2 interact to modulate Gata4 acetylation and embryonic cardiac myocyte proliferation. Developmental Cell. 19, 450-459 (2010).
35. N. Takeda et al., Interconversion between intestinal stem cell populations in distinct niches. Science. 334, 1420-1424 (2011).
36. N. Takeda et al., Hopx expression defines a subset of multipotent hair follicle stem cells and a progenitor population primed to give rise to K6+ niche cells. Development. 140, 1655-1664 (2013).
37. N. J. Palpant et al., Chromatin and transcriptional analysis of mesoderm progenitor cells identifies HOPX as a regulator of primitive hematopoiesis. Cell Reports. 20, 1597-1608 (2017).
38. L. Zhang et al., ZFP36L2 is required for self-renewal of early burst-forming unit erythroid progenitors. Nature. 499, 92-96 (2013).
39. M. D. Muzumdar, B. Tasic, K. Miyamichi, L. Li, L. Luo, A global double-fluorescent Cre reporter mouse. Genesis. 45, 593-605 (2007).
40. B. D. Harfe et al., Evidence for an expansion-based temporal Shh gradient in specifying vertebrate digit identities. Cell. 118, 517-528 (2004).
41. J. R. Prigge et al., Nuclear double-fluorescent reporter for in vivo and ex vivo analyses of biological transitions in mouse nuclei. Mammalian Genome. 24, 389-399 (2013).
42. A. Lyubimova et al., Single-molecule mRNA detection and counting in mammalian tissue. Nature Protocols. 8, 1743 (2013).
43. R. Satija, J. A. Farrell, D. Gennert, A. F. Schier, A. Regev, Spatial reconstruction of single-cell gene expression data. Nat. Biotechnol. 33, 495-502 (2015).
44. A. Butler, P. Hoffman, P. Smibert, E. Papalexi, R. Satija, Integrating single-cell transcriptomic data across different conditions, technologies, and species. Nat. Biotechnol. 36, 411-420 (2018).
45. T. Stuart et al., Comprehensive integration of single-cell data. Cell. 177, 1888-1902. e21 (2019).
46. C. Trapnell et al., The dynamics and regulators of cell fate decisions are revealed by pseudotemporal ordering of single cells. Nat. Biotechnol. 32, 381 (2014).
47. X. Qiu et al., Reversed graph embedding resolves complex single-cell trajectories. Nature Methods. 14, 979 (2017).
48. J. Cao et al., The single-cell transcriptional landscape of mammalian organogenesis. Nature. 566, 496-502 (2019).
49. L. McInnes, J. Healy, J. Melville, Umap: Uniform manifold approximation and projection for dimension reduction. arXiv Preprint arXiv: 1802.03426. (2018).
50. V. A. Traag, L. Waltman, N. J. Van Eck, From Louvain to Leiden: guaranteeing well-connected communities. Scientific Reports. 9, 1-12 (2019).
51. S. Hänzelmann, R. Castelo, J. Guinney, GSVA: gene set variation analysis for microarray and RNA-seq data. BMC Bioinformatics. 14, 1-15 (2013).
52. A. Liberzon et al., The molecular signatures database hallmark gene set collection. Cell Systems. 1, 417-425 (2015).

What is claimed is:

1. A method of expanding in culture an isolated population of pulmonary cells, the method comprising:
(a) dissociating a pulmonary tissue so as to obtain a population of isolated pulmonary cells; and
(b) expanding said population of isolated pulmonary cells in a medium comprising a factor that promotes expansion of endothelial cells, a factor that promotes expansion of epithelial cells and a factor that prevents differentiation, so as to expand a cell population being double positive for expression of epithelial and endothelial cell markers,
thereby expanding in culture the isolated population of pulmonary cells.

2. A method of qualifying suitability of an isolated population of pulmonary cells for administration to a subject in need thereof, the method comprising:
(a) dissociating a pulmonary tissue so as to obtain a population of isolated pulmonary cells;
(b) expanding said population of isolated pulmonary cells in a culture; and
(c) determining expression of epithelial and endothelial cell markers on said population of isolated pulmonary cells during and/or following said culture,
wherein expansion above a predetermined threshold of a cell population being double positive for expression of said epithelial and endothelial cell markers indicates said population of isolated pulmonary cells is suitable for administration to the subject; and
wherein no expansion or expansion below said predetermined threshold of said cell population being double positive for expression of said epithelial and endothelial cell markers indicates said population of isolated pulmonary cells is not suitable for administration to the subject,
thereby qualifying suitability of the isolated population of pulmonary cells for administration to the subject.

3. A method of generating an isolated population of pulmonary cells, the method comprising:
(a) dissociating a pulmonary tissue so as to obtain a population of isolated pulmonary cells; and
(b) contacting said population of isolated pulmonary cells with at least one agent capable of binding an epithelial cell marker and an endothelial cell marker, so as to select a cell population being double positive for expression of epithelial and endothelial cell markers,
thereby generating the isolated population of pulmonary cells.

4. The method of claim 3, further comprising expanding the pulmonary cells in a culture following step (b).

5. The method of claim 2, wherein said culture medium comprises a factor that promotes expansion of endothelial cells, a factor that promotes expansion of epithelial cells and a factor that prevents differentiation.

6. The method of claim 1, further comprising determining expression of said epithelial and endothelial cell markers on said pulmonary cells during and/or following said culture.

7. The method of claim 6,
wherein expansion above a predetermined threshold of said cell population being double positive for expression of said epithelial and endothelial cell markers indicates said population of isolated pulmonary cells is suitable for administration to a subject in need thereof; and
wherein no expansion or expansion below said predetermined threshold of said cell population being double positive for expression of said epithelial and endothelial cell markers indicates said population of isolated pulmonary cells is not suitable for administration to the subject.

8. The method of claim 1, wherein said factor that promotes expansion of endothelial cells is selected from the group consisting of vascular endothelial growth factor (VEGF), FGF, FGF2, IL-8 and BMP4.

9. The method of claim 1, wherein said factor that promotes expansion of endothelial cells comprises vascular endothelial growth factor (VEGF).

10. The method of claim 1, wherein said factor that promotes expansion of epithelial cells is selected from the group consisting of epidermal growth factor (EGF), Noggin and R-Spondin.

11. The method of claim 1, wherein said factor that promotes expansion of epithelial cells comprises epidermal growth factor (EGF).

12. The method of claim 1, wherein said factor that prevents differentiation is selected from the group consisting of a ROCK inhibitor, a GSK3b inhibitor and an ALK5 inhibitor.

13. The method of claim 1, wherein said factor that prevents differentiation comprises a ROCK inhibitor.

14. The method of claim 1, wherein said epithelial cell marker comprises CD326, CD324, CD24, Aquaporin 5 (AQP-5), Podoplanin (PDPN), or Advanced Glycosylation End-Product Specific Receptor (RAGE).

15. The method of claim 1, wherein said endothelial cell marker comprises CD31 or CD144 (VE-cadherin).

16. The method of claim 1, wherein said cell population being double positive for expression of epithelial and endothelial cell markers comprises a CD326⁺CD31⁺ signature.

17. The method of claim 1, further comprising depleting CD45 expressing cells.

18. An isolated population of pulmonary cells obtained according to the method of claim 1.

19. A method of regenerating an epithelial and/or endothelial pulmonary tissue in a subject in need thereof, the method comprising administering to the subject a therapeutically effective amount of the isolated population of pulmonary cells of claim 18, thereby regenerating the epithelial and/or endothelial pulmonary tissue.

20. A method of treating a pulmonary disorder or injury in a subject in need thereof, the method comprising administering to the subject a therapeutically effective amount of the isolated population of pulmonary cells of claim 18, thereby treating the pulmonary disorder or injury.
